(19) United States
(12) Patent Application Publication
(10) Pub. No.: US 2026/0028317 A1
(43) Pub. Date: Jan. 29, 2026

(54) EFFICIENT PROCEDURE FOR THE SYNTHESIS OF HYDRAZONE-LINKED TETRAHEDRAL NANOCAGES

(71) Applicant: UNIVERSITY OF VERMONT, Burlington, VT (US)

(72) Inventors: Severin T. SCHNEEBELI, West Lafayette, IN (US); Olav VESTRHEIM, Burlington, VT (US)

(21) Appl. No.: 18/994,393

(22) PCT Filed: Jul. 13, 2023
(86) PCT No.: PCT/US2023/027664

§ 371 Date: Jan. 14, 2025

Related U.S. Application Data
(60) Provisional application No. 63/389,435, filed on Jul. 15, 2022.

(51) Int. Cl. C07D 209/86; A61K 9/51; B82Y 5/00; B82Y 30/00; C07C 17/12

(52) U.S. Cl. CPC C07D 209/86; A61K 9/51; C07C 17/12; B82Y 5/00; B82Y 30/00

(57) ABSTRACT

The present application is directed to a nanocage of Formula (I): wherein A and R are as described herein. The present application is also directed to an efficient process for preparation of a nanocage of Formula (I), which allows for efficient scale-up of the nanocage synthesis. Furthermore, this new process allows for rapid nanocage diversification, due a newly introduced late-stage functionalization method.

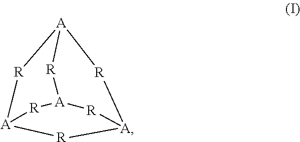

This application claims the priority benefit of U.S. Provisional Patent Application Ser. No. 63/389,435, filed Jul. 15, 2022, which is hereby incorporated by reference in its entirety.

This invention was made with government support under CHE-1848444 and CHE-2317652 awarded by National Science Foundation, under 2019-67022-29456 awarded by United States Department of Agriculture, and under 1R35GM147579 awarded by National Institute of General Medical Sciences. The government has certain rights in the invention.

### FIELD

The present application relates to an efficient procedure for the synthesis of hydrazone-linked tetrahedral nanocages.

### BACKGROUND

Molecular nanocages have gained a significant amount of attention in the last few decades as they can function as selective receptors and catalysts inspired by enzymes (Bols et al., “Template-Directed Synthesis of Molecular Nanorings and Cages,” Acc. Chem. Res. 51:2083 2092 (2018); Mastalerz, M., “Porous Shape-Persistent Organic Cage Compounds of Different Size, Geometry, and Function,” Acc. Chem. Res. 51:2411-2422 (2018); Acharyya et al., “Organic Imine Cages: Molecular Marriage and Applications,” Angew. Chem., Int. Ed. 58:8640 8653 (2019); Lauer et al., “Shape-Persistent [4+4] Imine Cages with a Truncated Tetrahedral Geometry,” Chem.-Eur. 1 24:1816-1820 (2018); Percastegui et al., “Design and Applications of Water-Soluble Coordination Cages,” Chem. Rev. 120:13480-13544 (2020); Yang et al., “Water-Stable Hydrazone-Linked Porous Organic Cages,” Chem. Sci. 12(40):13307-13315 (2021); Culshaw et al., “Dodecaamide Cages: Organic 12-Arm Building Blocks for Supramolecular Chemistry,” J. Am. Chem. Soc. 135:10007-10010 (2013); Hasell et al., “Molecular Doping of Porous Organic Cages,” J. Am. Chem. Soc. 133:14920-14923 (2011); Liu et al., “Acid- and Base-Stable Porous Organic Cages: Shape Persistence and pH Stability via Post-Synthetic “Tying” of a Flexible Amine Cage,” J. Am. Chem. Soc. 136:7583-7586 (2014); Mahata et al., “Giant Electroactive M4L6 Tetrahedral Host Self-Assembled with Fe(II) Vertices and Perylene Bisimide Dye Edges,” J. Am. Chem. Soc. 135:15656-15661 (2013); Nguyen et al., “Coordination Cages Selectively Transport Molecular Cargoes Across Liquid Membranes,” J. Am. Chem. Soc. 143:12175-12180 (2021); Zhang et al., “Vesicular Perylene Dye Nanocapsules as Supramolecular Fluorescent pH Sensor Systems,” Nat. Chem. 1:623-629 (2009); Greenaway et al., “High-Throughput Discovery of Organic Cages and Catenanes Using Computational Screening Fused with Robotic Synthesis,” Nat. Commun. 9:1-11 (2018); Hasell et al., “Porous Organic Cages: Soluble, Modular and Molecular Pores,” Nat. Rev. Mater. 1:16053 (2016); Fujita et al., “Self-Assembly of Tetravalent Goldberg Polyhedra from 144 Small Components,” Nature 540:563-566 (2016); Breslow, R., “Artificial Enzymes,” Science 218:532 (1982)). Many different porous organic cages (POCs) have been created in the last ten years, but one major drawback for nanocages is the often still limited stability in aqueous environments (Yang et al., “Water-Stable Hydrazone-Linked Porous Organic Cages,” Chem. Sci. 12(40):13307-13315 (2021); Sharafi et al., “Size-Selective Catalytic Polymer Acylation with a Molecular Tetrahedron,” Chem 6:1469-1494 (2020)). This is especially an issue with nanocages assembled using dynamic imine or boronate ester linkages, which have limited stability in water (Acharyya et al., “Organic Imine Cages: Molecular Marriage and Applications,” Angew. Chem., Int. Ed. 58:8640-8653 (2019); Lauer et al., “Shape-Persistent [4+4] Imine Cages with a Truncated Tetrahedral Geometry,” Chem.-Eur. 1 24:1816-1820 (2018); Yang et al., “Water-Stable Hydrazone-Linked Porous Organic Cages,” Chem. Sci. 12(40):13307-13315 (2021); Christinat et al., “Multi component Assembly of Boronic Acid Based Macrocycles and Cages,” Angew. Chem., Int. Ed. 47(10):1848-1852 (2008); Hutin et al., “An Iminoboronate Construction Set for Subcomponent Self-Assembly,” Chem.-Eur. 114(15):4585-4593 (2008); Icli et al., “Synthesis of Molecular Nanostructures by Multicomponent Condensation Reactions in a Ball Mill,” J. Am. Chem. Soc. 131(9):3154-3155 (2009); Rao et al., Boronic Acid Catalyzed Hydrolyses of Salicylaldehyde Imines,” J. Org. Chem. 56:1505 (1991)). Several strategies to access hydrolytically stable POCs have been developed, based on (i) alkyne metathesis (which leads to robust ethynylene linkages) (Lee et al., “Kinetically Trapped Tetrahedral Cages via Alkyne Metathesis,” J. Am. Chem. Soc. 138:2182-2185 (2016); Wang et al., “A Tetrameric Cage with D2h Symmetry Through Alkyne Metathesis,” Angew. Chem., Int. Ed. 53:10663-10667 (2014)), (ii) with hydrazone (Yang et al., “Water-Stable Hydrazone-Linked Porous Organic Cages,” Chem. Sci. 12(40):13307-13315 (2021); Wu et al., “A Self-Assembled Cage for Wide-Scope Chiral Recognition in Water,” Angew. Chem., Int. Ed. 60:16594-16599 (2021); Lin et al., “Multi component Assembly of Cavitand-Based Polyacylhydrazone Nanocapsules,” Chem.-Eur. 1 17:9395 (2011); Zheng et al., “Temperature-Dependent Self-Assembly of a Purely Organic Cage in Water,” Chem. Commun. 54:3138-3141 (2018); Li et al., “Quantitative Self-Assembly of a Purely Organic Three-Dimensional Catenane in Water,” Nat. Chem. 7:1003-1008 (2015)) oxime (Shen et al., “Dynamic Covalent Self-Assembly Based on Oxime Condensation,” Angew. Chem., Int. Ed. 57:16486-16490 (2018)) links, (iii) by converting imine bonds into more stable amide bonds (Culshaw et al., “Dodecaamide Cages: Organic 12-Arm Building Blocks for Supramolecular Chemistry,” J. Am. Chem. Soc. 135:10007-10010 (2013); Liu et al., “Acid- and Base-Stable Porous Organic Cages: Shape Persistence and pH Stability via Post-Synthetic “Tying” of a Flexible Amine Cage,” J. Am. Chem. Soc. 136:7583-7586 (2014); Martinez-Ahumada et al., “SO2 Capture Using Porous Organic Cages,” Angew. Chem., Int. Ed. 60:17556 17563 (2021); Martinez-Ahumada et al., “SO2 Capture Using Porous Organic Cages,” Angew. Chem., Int. Ed. 60:17556-17563 (2021), or (iv) by using irreversible carbon-carbon (Avellaneda et al., “Kinetically Controlled Porosity in a Robust Organic Cage Material,” Angew. Chem., Int. Ed. 52:3746-3749 (2013); Duan et al., “Host-Guest Recognition and Fluorescence of a Tetraphenylethene-Based Octacationic Cage,” Angew. Chem., Int. Ed. 59:10101-10110 (2020)) or carbon-oxygen (Zhang et al., “A Porous Tricyclooxacalixarene Cage Based on Tetraphenylethylene,” Angew. Chem., Int. Ed. 54:9244-9248 (2015)) bonds to form the nanocages, however, these efforts still lead to relatively small nanocages whose internal cavities are often not ideal for selective biomolecule recognition and polymerization catalysis.

The present application is directed to overcoming these and other deficiencies in the art.

### SUMMARY

One aspect of the present application relates to a nanocage of Formula (I):
wherein

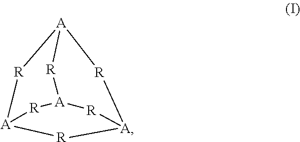

each A is independently selected and has the formula

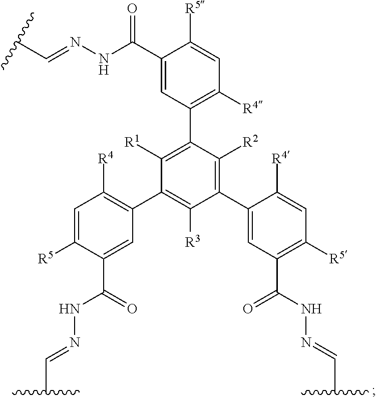

is the point of attachment of A to R;
each R is independently selected and has the formula

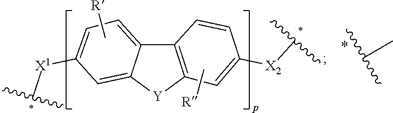

indicates the point of attachment of R to A; R′ is H, or C₁₋₂₀ alkyl;
R′ is H or C₁₋₂₀ alkyl;
R″ is H or C₁₋₂₀ alkyl;
R¹, R², and R³ are each independently selected from the group consisting of H, halogen, —OH, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁴, R^{4′}, and R^{4″} are each independently selected from the group consisting of H, halogen, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OH, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁵, R^{5′}, and R^{5″} are each independently selected from the group consisting of H, halogen, OH, —O(CH₂)ₙ—(OCH₂CH₂)ₘ—OC₁₋₆ alkyl, —O(CH₂)ₙ—(OCH₂CH₂)ₘ—OH, (OCH₂CH₂)ₘ—OC₁₋₆ alkyl, (OCH₂CH₂)ₘ—OH, aryl, heteroaryl, heterocyclyl, —OC₁₋₂₀ alkyl, —O-perfluorinated C₁₋₂₀alkyl, —Oaryl, and —NR¹⁰R¹¹;
R⁶ and R^{6′} are each independently selected at each occurrence from the group consisting of H, C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl, wherein C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl can be substituted from 1 to 3 times with a substituent selected from the group consisting of —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl;
R¹⁰ and R¹¹ are each independently selected from the group consisting of H, C₁₋₆ alkyl, C₂₋₆ alkenyl, C₂₋₆ alkynyl, aryl, heteroarylalkyl, heterocyclylalkyl, and arylalkyl, wherein each C₁₋₆ alkyl, C₂₋₆ alkenyl, C₂₋₆ alkynyl, aryl, heteroarylalkyl, heterocyclylalkyl, and arylalkyl can be optionally substituted from 1 to 3 times with a substituent selected from the group consisting of H, C₁₋₆ alkyl, heteroarylalkyl, heterocyclylalkyl, and arylalkyl;
X¹ and X² are absent or are independently selected from the group consisting of C₁₋₆ alkylene, C₃₋₈ cycloalkylene, and arylene, wherein C₁₋₆ alkylene, C₃₋₈ cycloalkylene, and arylene can be optionally substituted from 1 to 3 times with H or C₁₋₆ alkyl;
Y is independently selected from the group consisting of —N(R⁶)—, —S—, —O—, —C(R⁶)(R^{6′})—, and —P(R⁶)—;
p is 1 to 5;
n is 1 to 10; and
m is 1 to 50.

Another aspect of the present application relates to a process for preparation of a nanocage of Formula (I):

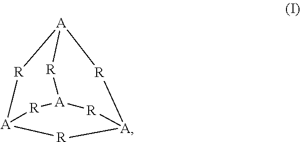

each A is independently selected and has the formula

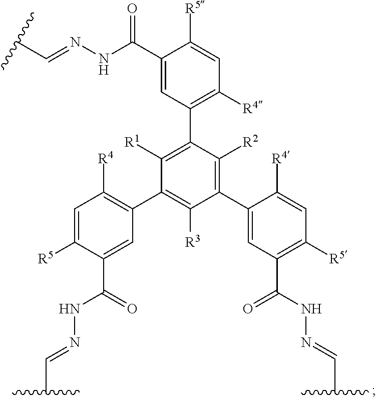

is the point of attachment of A to R;
each R is independently selected and has the formula

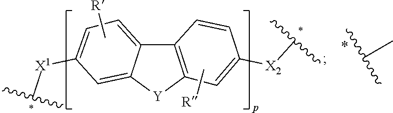

indicates the point of attachment of R to A;
R′ is H or C₁₋₂₀ alkyl;
R″ is H or C₁₋₂₀ alkyl;
R¹, R², and R³ are each independently selected from the group consisting of H, halogen, —OH, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁴, R^{4′}, and R^{4″} are each independently selected from the group consisting of H, halogen, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OH, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁵, R^{5′}, and R^{5″} are each independently selected from the group consisting of H, halogen, —OH, —O(CH₂)ₙ—(OCH₂CH₂)ₘ—OC₁₋₆ alkyl, —O(CH₂)ₙ—(OCH₂CH₂)ₘ—OH, (OCH₂CH₂)ₘ—OC₁₋₆ alkyl, (OCH₂CH₂)ₘ—OH, aryl, heteroaryl, heterocyclyl, —OC₁₋₂₀ alkyl, —O-perfluorinated C₁₋₂₀ alkyl, —Oaryl, and —NR¹⁰R¹¹;
R⁶ and R^{6′} are each independently selected at each occurrence from the group consisting of H, C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl, wherein C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl can be substituted from 1 to 3 times with a substituent selected from the group consisting of —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl;
R¹⁰ and R¹¹ are each independently selected from the group consisting of H, C₁₋₆ alkyl, C₂₋₆ alkenyl, C₂₋₆ alkynyl, aryl, heteroarylalkyl, heterocyclylalkyl, and arylalkyl, wherein each C₁₋₆ alkyl, C₂₋₆ alkenyl, C₂₋₆ alkynyl, aryl, heteroarylalkyl, heterocyclylalkyl, and arylalkyl can be optionally substituted from 1 to 3 times with a substituent selected from the group consisting of H, C₁₋₆ alkyl, heteroarylalkyl, heterocyclylalkyl, and arylalkyl;
X¹ and X² are absent or are independently selected from the group consisting of C₁₋₆ alkylene, C₃₋₈ cycloalkylene, and arylene, wherein C₁₋₆ alkylene, C₃₋₈ cycloalkylene, and arylene can be optionally substituted from 1 to 3 times with H or C₁₋₆ alkyl;
Y is independently selected from the group consisting of —N(R⁶)—, —S—, —O—, —C(R⁶)(R^{6′})—, and —P(R⁶)—;
p is 1 to 5;
n is 1 to 10; and
m is 1 to 50, said process comprising:
providing one or more compounds of Formula (II) having the structure:

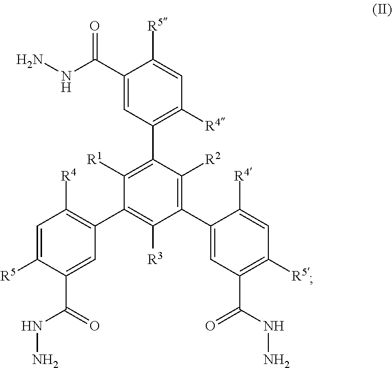

and
forming the nanocage of Formula (I) from the one or more compounds of Formula (II).

Another aspect of the present application relates to a process for preparation of a nanocage of Formula (I′):

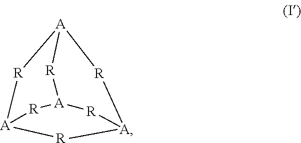

wherein
each A is independently selected and has the formula

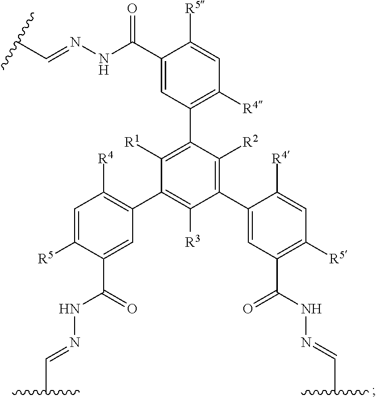

is the point of attachment of A to R;

each R is independently selected and has the formula

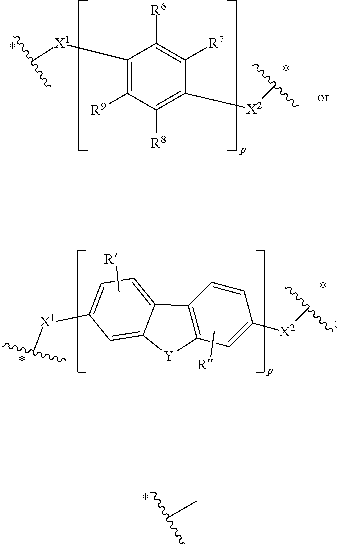

indicates the point of attachment of R to A;
R′ is H or C₁₋₂₀ alkyl;
R″ is H or C₁₋₂₀ alkyl;
R¹, R², and R³ are each independently selected from the group consisting of H, halogen, —OH, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁴, R^{4′}, and R^{4″} are each independently selected from the group consisting of H, halogen, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OH, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁵, R^{5′}, and R^{5″} are each independently selected from the group consisting of H, halogen, —OH, —O(CH₂)ₙ—(OCH₂CH₂)ₘ—OC₁₋₆ alkyl, —O(CH₂)ₙ—(OCH₂CH₂)ₘ—OH, (OCH₂CH₂)ₘ—OC₁₋₆ alkyl, (OCH₂CH₂)ₘ—OH, aryl, heteroaryl, heterocyclyl, —OC₁₋₂₀ alkyl, —O-perfluorinated C₁₋₂₀alkyl, —Oaryl, and —NR¹⁰R¹¹;
R^{6′} and R^{6″} are each independently selected at each occurrence from the group consisting of H, C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl, wherein C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl can be substituted from 1 to 3 times with a substituent selected from the group consisting of —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl;
R⁶, R⁷, R⁸, and R⁹ are each independently selected at each occurrence from the group consisting of H, halogen, C₁₋₂₀ alkyl, C₂₋₂₀ alkenyl, C₂₋₂₀ alkynyl, perfluorinated C₁₋₂₀ alkyl, aryl, heteroaryl, heterocyclyl, OH, —OC₁₋₂₀ alkyl, —OC₂₋₂₀ alkenyl, —O-perfluorinated C₁₋₂₀ alkyl, —OC₂₋₂₀ alkynyl, aryl, heteroaryl, heterocyclyl, and —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, CONH perfluorinated C₁₋₂₀ alkyl, —CONH-aryl, —CONH-heteroaryl, and —CONH— heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl can be optionally substituted from 1 to 3 times with a substituent selected from the group consisting of H, halogen, OH, C₁₋₆ alkyl, aryl, and arylalkyl;

R¹⁰ and R¹¹ are each independently selected from the group consisting of H, C₁₋₆ alkyl, C₂₋₆ alkenyl, C₂₋₆ alkynyl, aryl, heteroarylalkyl, heterocyclylalkyl, and arylalkyl, wherein each C₁₋₆ alkyl, C₂₋₆ alkenyl, C₂₋₆ alkynyl, aryl, heteroarylalkyl, heterocyclylalkyl, and arylalkyl can be optionally substituted from 1 to 3 times with a substituent selected from the group consisting of H, C₁₋₆ alkyl, heteroarylalkyl, heterocyclylalkyl, and arylalkyl;

X¹ and X² are absent or are independently selected from the group consisting of C₁₋₆ alkylene, C₃₋₈ cycloalkylene, and arylene, wherein C₁₋₆ alkylene, C₃₋₈ cycloalkylene, and arylene can be optionally substituted from 1 to 3 times with H or C₁₋₆ alkyl;
Y is independently selected from the group consisting of —N(R^{6′})—, —S—, —O—, —C(R^{6′})(R^{6″})—, and —P(R⁶)—;
p is 1 to 5;
n is 1 to 10; and
m is 1 to 50, said process comprising:
providing one or more compounds of Formula (V′) having the structure:

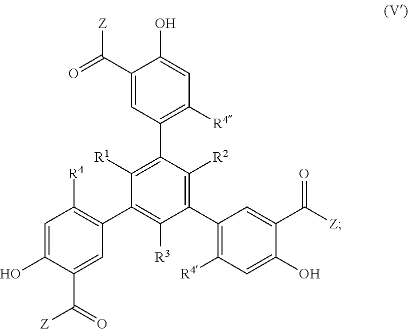

wherein Z is —O—C₁₋₆ alkyl; and
forming the nanocage of Formula (I′) from the one or more compounds of Formula (V′).

Another aspect of the present application relates to a method for detecting an analyte in a fluid. This method includes:
providing a sensor comprising a nanocage of Formula (I):

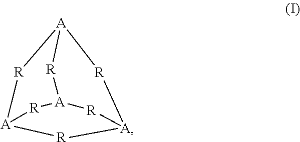

wherein
each A is independently selected and has the formula

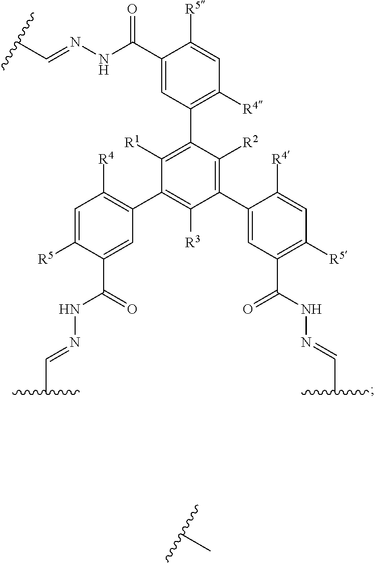

is the point of attachment of A to R;
each R is independently selected and has the formula

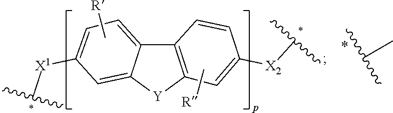

indicates the point of attachment of R to A; R
R′ is H or C₁₋₂₀ alkyl;
R″ is H or C₁₋₂₀ alkyl; p
R¹, R², and R³ are each independently selected from the group consisting of H, halogen, OH, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;

R⁴, R^{4′}, and R^{4″} are each independently selected from the group consisting of H, halogen, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OH, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀-alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl; R⁵, R^{5′}, and R^{5″} are each independently selected from the group consisting of H, halogen, —OH, —O(CH₂)ₙ—(OCH₂CH₂)ₘ—OC₁₋₆ alkyl, —O(CH₂)ₙ—(OCH₂CH₂)ₘ—OH, (OCH₂CH₂)ₘ—OC₁₋₆ alkyl, (OCH₂CH₂)ₘ—OH, aryl, heteroaryl, heterocyclyl, —OC₁₋₂₀ alkyl, —O-perfluorinated C₁₋₂₀ alkyl, —Oaryl, and —NR¹⁰R¹¹;
R⁶ and R^{6′} are each independently selected at each occurrence from the group consisting of H, C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl, wherein C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl can be substituted from 1 to 3 times with a substituent selected from the group consisting of —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀-alkynyl, —CONH perfluorinated C₁₋₂₀ alkyl, —CONH-aryl, —CONH-heteroaryl, and CONH-heterocyclyl;

R¹⁰ and R¹¹ are each independently selected from the group consisting of H, C₁₋₆ alkyl, C₂₋₆ alkenyl, C₂₋₆ alkynyl, aryl, heteroarylalkyl, heterocyclylalkyl, and arylalkyl, wherein each C₁₋₆ alkyl, C₂₋₆ alkenyl, C₂₋₆ alkynyl, aryl, heteroarylalkyl, heterocyclylalkyl, and arylalkyl can be optionally substituted from 1 to 3 times with a substituent selected from the group consisting of H, C₁₋₆ alkyl, heteroarylalkyl, heterocyclylalkyl, and arylalkyl;
X¹ and X² are absent or are independently selected from the group consisting of C₁₋₆ alkylene, C₃₋₈ cycloalkylene, and arylene, wherein C₁₋₆ alkylene, C₃₋₈ cycloalkylene, and arylene can be optionally substituted from 1 to 3 times with H or C₁₋₆ alkyl;
Y is independently selected from the group consisting of —N(R⁶)—, —S—, —O—, —C(R⁶)(R^{6′})—, and —P(R⁶)—;
p is 1 to 5;
n is 1 to 10; and
m is 1 to 50;
providing a fluid containing an analyte; and
contacting a fluid containing the analyte with the sensor to capture the analyte in the nanocage and detect the analyte in the fluid.

Another aspect of the present application relates to a method of functionalizing a polymer. This method includes:
providing a polymer;
providing a nanocage of Formula (I):

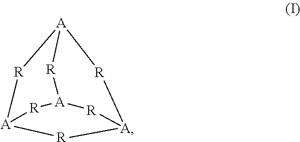

wherein each A is independently selected and has the formula

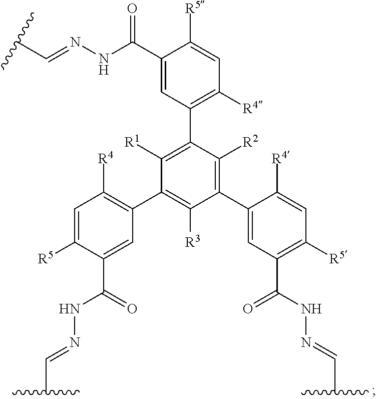

is the point of attachment of A to R;
each R is independently selected and has the formula

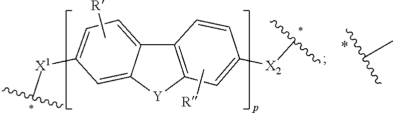

indicates the point of attachment of R to A;
R′ is H or C₁₋₂₀ alkyl;
R″ is H or C₁₋₂₀ alkyl;
R¹, R², and R³ are each independently selected from the group consisting of H, halogen, —OH, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁴, R^{4′}, and R^{4″} are each independently selected from the group consisting of H, halogen, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OH, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁵, R^{5′}, and R^{5″} are each independently selected from the group consisting of H, halogen, OH, —O(CH₂)ₙ—(OCH₂CH₂)ₘ—OC₁₋₆ alkyl, —O(CH₂)ₙ—(OCH₂CH₂)ₘ—OH, (OCH₂CH₂)ₘ—OC₁₋₆ alkyl, (OCH₂CH₂)ₘ—OH, aryl, heteroaryl, heterocyclyl, —OC₁₋₂₀ alkyl, —O-perfluorinated C₁₋₂₀ alkyl, —Oaryl, and —NR¹⁰R¹¹;
R⁶ and R^{6′} are each independently selected at each occurrence from the group consisting of H, C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl, wherein C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl can be substituted from 1 to 3 times with a substituent selected from the group consisting of —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl;
R¹⁰ and R¹¹ are each independently selected from the group consisting of H, C₁₋₆ alkyl, C₂₋₆ alkenyl, C₂₋₆ alkynyl, aryl, heteroarylalkyl, heterocyclylalkyl, and arylalkyl, wherein each C₁₋₆ alkyl, C₂₋₆ alkenyl, C₂₋₆ alkynyl, aryl, heteroarylalkyl, heterocyclylalkyl, and arylalkyl can be optionally substituted from 1 to 3 times with a substituent selected from the group consisting of H, C₁₋₆ alkyl, heteroarylalkyl, heterocyclylalkyl, and arylalkyl;
X¹ and X² are absent or are independently selected from the group consisting of C₁₋₆ alkylene, C₃₋₈ cycloalkylene, and arylene, wherein C₁₋₆ alkylene, C₃₋₈ cycloalkylene, and arylene can be optionally substituted from 1 to 3 times with H or C₁₋₆ alkyl;
Y is independently selected from the group consisting of —N(R⁶)—, —S—, —O—, —C(R⁶)(R^{6′})—, and —P(R⁶)—;
p is 1 to 5;
n is 1 to 10; and
m is 1 to 50;
providing a functionalizing reagent;
reacting the polymer with the functionalizing reagent within the nanocage having a Formula (I) to produce a functionalized polymer.

The present application describes the creation of large, covalent tetrahedral nanocages with hydrazone links, which show superior hydrolytic stability, and can also act as excellent hydrogen bonding acceptors to enable selective polymerization catalysis inside the nanocages (Sharafi et al., “Size-Selective Catalytic Polymer Acylation with a Molecular Tetrahedron,” Chem 6:1469-1494 (2020), which is hereby incorporated by reference in its entirety).

The present application describes an efficient synthetic procedure for the synthesis of a robust, hydrazone-linked molecular tetrahedron with large internal cavities, enabling creation of multiple grams of the tetrahedral nanocages. This synthetic approach involves a late-stage functionalization utilized to install the peripheral functional groups protruding from the tetrahedron, which opens up the possibility to readily access a large family of related hydrazone-linked tetrahedra with tunable surface properties for sensing and catalysis.

The synthetic procedure described in the present application represents a great improvement, and it is highly scalable. Notably, only a single chromatographic purification step is needed, which provides a practical path to gram-scale quantities of the tetrahedron nanocages. Furthermore, this new synthesis also provides for an efficient late-stage functionalization approach (FIG. 1), which will allow to readily synthesize a large family of related, hydrazone-linked nanocages.

### BRIEF DESCRIPTION OF THE DRAWINGS

FIG. 1 is a schematic showing new, scalable synthesis, which allows to readily access a family of large hydrazone-linked tetrahedral nanocages with different peripheral functional groups.

FIG. 2 are images showing compound 2 before washing with acetone (left) and compound 2 after washing with acetone (right).

FIG. 3 are images showing suspension of compound 3 (mixture of syn- and anti-atropodiastereoisomers) immediately after pouring the reaction mixture into water (left) and suspension and filtration after 24 hours (right).

FIG. 4 are images showing hot wash of crude syn-3 (left) and precipitation of syn-4 after stirring the reaction mixture in 3 L water for 24 hours (right).

FIG. 5 are images showing precipitation of syn-5 after 24 hours of stirring as an aqueous suspension (left) and reaction setup to form syn-9 (right).

FIG. 6 shows the synthesis of hydrazone-linked tetrahedron 10.

FIG. 7 shows a newly developed synthetic procedure for the hydrazone-linked molecular tetrahedron 10.

FIG. 8 shows the structure of the hydrazone-linked tetrahedron 100.

FIG. 9 shows ¹H NMR spectrum (500 MHz, CDCl₃, 298 K) of syn-3.

FIG. 10 shows ¹³C (¹H) NMR spectrum (125 MHz, CDCl₃, 298 K) of syn-3.

FIG. 11 shows ¹H NMR spectrum (500 MHz, CDCl₃, 298 K) of syn-4.

FIG. 12 shows ¹³C (¹H) NMR spectrum (125 MHz, CDCl₃, 298 K) of syn-4.

FIG. 13 shows ¹H NMR spectrum (500 MHz, DMSO-d₆, 298 K) of syn-5.

FIG. 14 shows ¹³C (¹H) NMR spectrum (125 MHz, DMSO-d₆, 298 K) of syn-5.

FIG. 15 shows ¹H NMR spectrum (500 MHz, CDCl₃, 298 K) of syn-6.

FIG. 16 shows ¹³C (¹H) NMR spectrum (125 MHz, CDCl₃, 298 K) of syn-6.

FIG. 17 shows ¹H NMR spectrum (500 MHz, CDCl₃, 298 K) of syn-7.

FIG. 18 shows ¹³C (¹H) NMR spectrum (125 MHz, CDCl₃, 298 K) of syn-7.

FIG. 19 shows ¹H NMR spectrum (500 MHz, CDCl₃, 298 K) of syn-8.

FIG. 20 shows ¹³C (¹H) NMR spectrum (125 MHz, CDCl₃, 298 K) of syn-8.

FIG. 21 shows ¹H NMR spectrum (500 MHz, CDCl₃, 298 K) of syn-9.

FIG. 22 shows ¹³C (¹H) NMR spectrum (125 MHz, CDCl₃, 298 K) of syn-9.

FIG. 23 shows ¹H NMR spectrum (500 MHz, CD₂Cl₂, 298 K) of hydrazone-linked tetrahedron 10.

FIG. 24 shows ¹³C (¹H) NMR spectrum (125 MHz, CDCl₃, 298 K) of hydrazone-linked tetrahedron 10.

FIG. 25 shows ¹H NMR spectrum (800 MHz, CD₂Cl₂, 298 K) of hydrazone-linked tetrahedron 11.

FIG. 26 shows ¹³C (¹H) NMR spectrum (200 MHz, CDCl₃, 298 K) of hydrazone-linked tetrahedron 11.

FIGS. 27A-B show full (FIG. 27A) and partial (FIG. 27B)¹H NMR spectrum (800 MHz, C CD₂Cl₂, 298 K) of hydrazone-linked tetrahedron 12.

FIG. 28 shows ¹³C (¹H) NMR spectrum (200 MHz, CDCl₃, 298 K) of hydrazone-linked tetrahedron 12.

FIG. 29 shows ¹H NMR spectrum (800 MHz, CD₂Cl₂, 298 K) of hydrazone-linked tetrahedron 13.

FIG. 30 shows ¹³C (¹H) NMR spectrum (200 MHz, CDCl₃, 298 K) of hydrazone-linked tetrahedron 13.

FIG. 31 shows the synthesis of hydrazone-linked tetrahedron 11.

FIG. 32 shows the synthesis of hydrazone-linked tetrahedron 12.

FIG. 33 shows the synthesis of hydrazone-linked tetrahedron 13.

FIG. 34 shows the synthesis of three new hydrazone-linked covalent cages, 11 with a biphenyl linker, 12 with ap-terphenyl linker, and 13 with ap-quarterphenyl linker. R′═—(CH₂)₄(OCH₂CH₂)₃OCH₃. R=—(CH₂)₃(OCH₂CH₂)₃OCH₃. The structures of the molecular cages represent DFT-optimized models with R=—H.

FIG. 35 shows the HRMS (ESI+) spectrum of hydrazone-linked tetrahedron 11.

FIG. 36 shows the isotopic distribution of the [M+5H]⁵⁺ peak of the HRMS (ESI+) spectrum of hydrazone-linked tetrahedron 11.

FIG. 37 shows the isotopic distribution of the [M+6H]⁶⁺ peak of the HRMS (ESI+) spectrum of hydrazone-linked tetrahedron 11.

FIG. 38 shows the MALDI-TOF mass spectrum (DCTB matrix) of hydrazone-linked tetrahedron 12.

FIG. 39 shows the partial MALDI-TOF mass spectrum (DCTB matrix) of hydrazone-linked tetrahedron 12.

FIG. 40 shows the MALDI-TOF mass spectrum (DCTB matrix) of hydrazone-linked tetrahedron 13.

FIG. 41 shows the partial MALDI-TOF mass spectrum (DCTB matrix) of hydrazone-linked tetrahedron 13.

FIG. 42 shows the DFT-optimized structure of the p-quarterphenyl-linked molecular cage 13. The sphere illustrating the cavity of the cage measures 3.2 nm in diameter, with a sphere volume of 17 nm³. R=—(CH₂)₃(OCH₂CH₂)₃OCH₃ in the synthesized structure, while R=—H in the DFT model.

FIG. 43 shows ¹H DOSY NMR spectrum (800 MHz, CD₂Cl₂) of hydrazone-linked tetrahedron 10.

FIG. 44 shows ¹H DOSY NMR spectrum (800 MHz, CD₂Cl₂) of hydrazone-linked tetrahedron 11.

FIG. 45 shows ¹H DOSY NMR spectrum (800 MHz, CD₂Cl₂) of hydrazone-linked tetrahedron 12.

FIG. 46 shows ¹H DOSY NMR spectrum (800 MHz, CD₂Cl₂) of hydrazone-linked tetrahedron 13.

FIG. 47 shows an intrinsic reaction coordinate scan for the inversion of syn-9 to anti-9, obtained at the B3LYP/LACVP* level of theory. At the B3LYP/aug-cc-PVDZ//B3LYP/LACVP* level of theory, the activation energy for syn-9 to anti-9 conversion was calculated to be 28.5 kcal/mol, which explains why the syn-configuration of the vertex is stable, even under mild heating.

### DETAILED DESCRIPTION

One aspect of the present application relates to a nanocage of Formula (I):

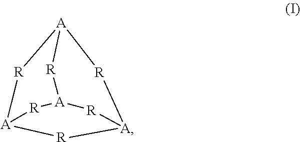

wherein
each A is independently selected and has the formula

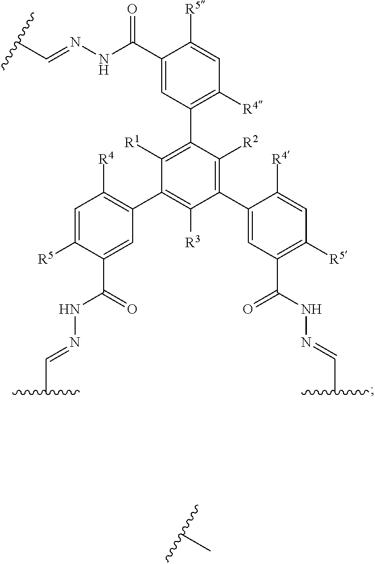

is the point of attachment of A to R;
each R is independently selected and has the formula

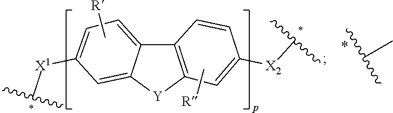

indicates the point of attachment of R to A; R′ is H, or C₁₋₂₀ alkyl;
R′ is H or C₁₋₂₀ alkyl;
R″ is H or C₁₋₂₀ alkyl;
R¹, R², and R³ are each independently selected from the group consisting of H, halogen, OH, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀-alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;

R⁴, R^{4′}, and R^{4″} are each independently selected from the group consisting of H, halogen, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OH, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀-alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁵, R^{5′}, and R^{5″} are each independently selected from the group consisting of H, halogen, —OH, —O(CH₂)ₙ—(OCH₂CH₂)ₘ—OC₁₋₆ alkyl, —O(CH₂)ₙ—(OCH₂CH₂)ₘ—OH, (OCH₂CH₂)ₘ—OC₁₋₆ alkyl, (OCH₂CH₂)ₘ—OH, aryl, heteroaryl, heterocyclyl, —OC₁₋₂₀ alkyl, —O-perfluorinated C₁₋₂₀ alkyl, —Oaryl, and —NR¹⁰R¹¹;
R⁶ and R^{6′} are each independently selected at each occurrence from the group consisting of H, C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl, wherein C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl can be substituted from 1 to 3 times with a substituent selected from the group consisting of —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀-alkynyl, —CONH perfluorinated C₁₋₂₀ alkyl, —CONH-aryl, —CONH-heteroaryl, and CONH-heterocyclyl;

R¹⁰ and R¹¹ are each independently selected from the group consisting of H, C₁₋₆ alkyl, C₂₋₆ alkenyl, C₂₋₆ alkynyl, aryl, heteroarylalkyl, heterocyclylalkyl, and arylalkyl, wherein each C₁₋₆ alkyl, C₂₋₆ alkenyl, C₂₋₆ alkynyl, aryl, heteroarylalkyl, heterocyclylalkyl, and arylalkyl can be optionally substituted from 1 to 3 times with a substituent selected from the group consisting of H, C₁₋₆ alkyl, heteroarylalkyl, heterocyclylalkyl, and arylalkyl;
X¹ and X² are absent or are independently selected from the group consisting of C₁₋₆ alkylene, C₃₋₈ cycloalkylene, and arylene, wherein C₁₋₆ alkylene, C₃₋₈ cycloalkylene, and arylene can be optionally substituted from 1 to 3 times with H or C₁₋₆ alkyl;
Y is independently selected from the group consisting of —N(R⁶)—, —S—, —O—, —C(R⁶)(R^{6′})—, and —P(R⁶)—;
p is 1 to 5;
n is 1 to 10; and
m is 1 to 50.

As used above, and throughout the description herein, the following terms, unless otherwise indicated, shall be understood to have the following meanings. If not defined otherwise herein, all technical and scientific terms used herein have the same meaning as is commonly understood by one of ordinary skill in the art to which this technology belongs. In the event that there is a plurality of definitions for a term herein, those in this section prevail unless stated otherwise.

The term “alkyl” means an aliphatic hydrocarbon group which may be straight or branched. When not otherwise restricted, the term refers to an alkyl of 20 or fewer carbons. Lower alkyl refers to alkyl groups having about 1 to about 6 carbon atoms in the chain. Branched means that one or more lower alkyl groups such as methyl, ethyl, or propyl are attached to a linear alkyl chain. Exemplary alkyl groups include methyl, ethyl, n-propyl, i-propyl, n-butyl, t-butyl, n-pentyl, 3-pentyl, and the like.

The term “alkenyl” means an aliphatic hydrocarbon group containing a carbon-carbon double bond and which may be straight or branched having about 2 to about 20 carbon atoms in the chain. Particular alkenyl groups have 2 to about 10 carbon atoms in the chain. Branched means that one or more lower alkyl groups such as methyl, ethyl, or propyl are attached to a linear alkenyl chain. Exemplary alkenyl groups include ethenyl, propenyl, n-butenyl, and i-butenyl. The term “alkenyl” may also refer to a hydrocarbon chain having 2 to 6 carbons containing at least one double bond and at least one triple bond.

The term “alkynyl” means an aliphatic hydrocarbon group containing a carbon-carbon triple bond and which may be straight or branched having about 2 to about 20 carbon atoms in the chain. Particular alkynyl groups have 2 to about 10 carbon atoms in the chain. Branched means that one or more lower alkyl groups such as methyl, ethyl, or propyl are attached to a linear alkynyl chain. Exemplary alkynyl groups include ethynyl, propynyl, n-butynyl, 2-butynyl, 3-methylbutynyl, and n-pentynyl.

The term “cycloalkyl” means a non-aromatic mono- or multicyclic ring system of about 3 to about 12 carbon atoms, preferably of about 3 to about 8 carbon atoms. Exemplary monocyclic cycloalkyls include cyclopentyl, cyclohexyl, cycloheptyl, bicyclo[1.1.1]pentyl, and the like.

As used herein, the term “alkane” refers to aliphatic hydrocarbons of formula CₙH₂ₙ₊₂, which may be straight or branched having about 1 to about 40 (e.g., 1-2, 1-3, 1-4, 1-5, 1-6, 1-7, 1-8) carbon atoms in the chain. Branched means that one or more lower alkyl groups such as methyl, ethyl, or propyl are attached to a linear alkyl chain. Exemplary alkanes include methane, ethane, n-propane, i-propane, n-butane, t-butane, n-pentane, and 3-pentane. The term alkylene” refers to a divalent group formed from an alkane by removal of two hydrogen atoms. Exemplary alkylene groups include, but are not limited to, divalent groups derived from the alkanes described above.

As used herein, the term “cycloalkane” refers to aliphatic hydrocarbons of formula CₙH₂ₙ, which may be straight or branched having about 3 to about 8 carbon atoms in the chain. Exemplary cycloalkanes include cyclopropane, cyclobutane, cyclopentane, cyclohexane, and cycloheptane. The term “cycloalkylene” refers to a divalent group formed from a cycloalkane by removal of two hydrogen atoms. Exemplary cycloalkylene groups include, but are not limited to, divalent groups derived from the cycloalkanes described above.

The term “monocyclic” used herein indicates a molecular structure having one ring.

The term “polycyclic” or “multi-cyclic” used herein indicates a molecular structure having two or more rings, including, but not limited to, fused, bridged, or spiro rings.

The term “aryl” means an aromatic monocyclic or multi-cyclic (polycyclic) ring system of 6 to about 19 carbon atoms, or of 6 to about 10 carbon atoms, and includes arylalkyl groups. The ring system of the aryl group may be optionally substituted. Representative aryl groups include, but are not limited to, groups such as phenyl, naphthyl, azulenyl, phenanthrenyl, anthracenyl, fluorenyl, pyrenyl, triphenylenyl, chrysenyl, and naphthacenyl.

The term “heteroaryl” means an aromatic monocyclic or multi-cyclic ring system of about 5 to about 19 ring atoms, or about 5 to about 10 ring atoms, in which one or more of the atoms in the ring system is/are element(s) other than carbon, for example, nitrogen, oxygen, or sulfur. In the case of multi-cyclic ring system, only one of the rings needs to be aromatic for the ring system to be defined as “heteroaryl”. Particular heteroaryls contain about 5 to 6 ring atoms. The prefix aza, oxa, thia, or thio before heteroaryl means that at least a nitrogen, oxygen, or sulfur atom, respectively, is present as a ring atom. A nitrogen, carbon, or sulfur atom in the heteroaryl ring may be optionally oxidized; the nitrogen may optionally be quaternized. Representative heteroaryls include pyridyl, 2-oxo-pyridinyl, pyrimidinyl, pyridazinyl, pyrazinyl, triazinyl, furanyl, pyrrolyl, thiophenyl, pyrazolyl, imidazolyl, oxazolyl, isoxazolyl, thiazolyl, isothiazolyl, triazolyl, oxadiazolyl, thiadiazolyl, tetrazolyl, indolyl, isoindolyl, benzofuranyl, benzothiophenyl, indolinyl, 2-oxoindolinyl, dihydrobenzofuranyl, dihydrobenzothiophenyl, indazolyl, benzimidazolyl, benzooxazolyl, benzothiazolyl, benzoisoxazolyl, benzoisothiazolyl, benzotriazolyl, benzo[1,3]dioxolyl, quinolinyl, isoquinolinyl, quinazolinyl, cinnolinyl, pthalazinyl, quinoxalinyl, 2,3-dihydro-benzo[1,4]dioxinyl, benzo[1,2,3]triazinyl, benzo[1,2,4]triazinyl, 4H-chromenyl, indolizinyl, quinolizinyl, 6aH-thieno[2,3-d]imidazolyl, 1H-pyrrolo[2,3-b]pyridinyl, imidazo[1,2-a]pyridinyl, pyrazolo[1,5-a]pyridinyl, [1,2,4]triazolo[4,3-a]pyridinyl, [1,2,4]triazolo[1,5-a]pyridinyl, thieno[2,3-b]furanyl, thieno[2,3-b]pyridinyl, thieno[3,2-b]pyridinyl, furo[2,3-b]pyridinyl, furo[3,2-b]pyridinyl, thieno[3,2-d]pyrimidinyl, furo[3,2-d]pyrimidinyl, thieno[2,3-b]pyrazinyl, imidazo[1,2-a]pyrazinyl, 5,6,7,8-tetrahydroimidazo[1,2-a]pyrazinyl, 6,7-dihydro-4H-pyrazolo[5,1-c][1,4]oxazinyl, 2-oxo-2,3-dihydrobenzo[d]oxazolyl, 3,3-dimethyl-2-oxoindolinyl, 2-oxo-2,3-dihydro-1H-pyrrolo[2,3-b]pyridinyl, benzo[c][1,2,5]oxadiazolyl, benzo[c][1,2,5]thiadiazolyl, 3,4-dihydro-2H-benzo[b][1,4]oxazinyl, 5,6,7,8-tetrahydro-[1,2,4]triazolo[4,3-a]pyrazinyl, [1,2,4]triazolo[4,3-a]pyrazinyl, 3-oxo-[1,2,4]triazolo[4,3-a]pyridin-2(3H)-yl, and the like.

As used herein, “heterocyclyl” or “heterocycle” refers to a stable 3- to 18-membered ring (radical) which consists of carbon atoms and from one to five heteroatoms selected from the group consisting of nitrogen, oxygen, and sulfur. For purposes of this application, the heterocycle may be a monocyclic, or a polycyclic ring system, which may include fused, bridged, or spiro ring systems; and the nitrogen, carbon, or sulfur atoms in the heterocycle may be optionally oxidized; the nitrogen atom may be optionally quaternized; and the ring may be partially or fully saturated. Examples of such heterocycles include, without limitation, azepinyl, azocanyl, pyranyl dioxanyl, dithianyl, 1,3-dioxolanyl, tetrahydrofuryl, dihydropyrrolidinyl, decahydroisoquinolyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, 2-oxoazepinyl, oxazolidinyl, oxiranyl, piperidinyl, piperazinyl, 4-piperidonyl, pyrrolidinyl, pyrazolidinyl, thiazolidinyl, tetrahydropyranyl, thiamorpholinyl, thiamorpholinyl sulfoxide, and thiamorpholinyl sulfone. Further heterocycles and heteroaryls are described in Katritzky et al., eds., Comprehensive Heterocyclic Chemistry: The Structure, Reactions, Synthesis and Use of Heterocyclic Compounds, Vol. 1-8, Pergamon Press, N.Y. (1984), which is hereby incorporated by reference in its entirety.

The terms “arylalkyl” and “heteroarylalkyl” mean an alkyl substituted with one or more aryl or heteroaryl groups, wherein the alkyl, aryl, and heteroaryl groups are as herein described. One particular example is an arylmethyl or heteroarylmethyl group, in which a single carbon spacer unit is attached to an aryl or heteroaryl group, where the carbon spacer and the aryl or heteroaryl group can be optionally substituted as described herein.

The term “heterocyclylalkyl” mean an alkyl substituted with one or more heterocyclyl groups, wherein the alkyl and heterocyclyl groups are as herein described.

The term “arylene” means a group obtained by removal of a hydrogen atom from an aryl group. Non-limiting examples of arylene include phenylene and naphthylene.

The term “halogen” means fluoro, chloro, bromo, or iodo.

The term “perfluorinated C₁₋₂₀ alkyl” means both branched and straight-chain alkyl substituted with one or more fluorine atoms, wherein the alkyl group is as herein described.

The term “optionally substituted” is used to indicate that a group may have a substituent at each substitutable atom of the group (including more than one substituent on a single atom), provided that the designated atom's normal valency is not exceeded, and the identity of each substituent is independent of the others. Up to three H atoms in each residue are replaced with alkyl, halogen, haloalkyl, hydroxy, loweralkoxy, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy. “Unsubstituted” atoms bear all of the hydrogen atoms dictated by their valency. When a substituent is keto (i.e., =0), then two hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture, and formulation into an efficacious therapeutic agent.

The term “a derivative thereof” refers to a salt thereof, an ester thereof, a free acid form thereof, a free base form thereof, a solvate thereof, a deuterated derivative thereof, a hydrate thereof, an N-oxide thereof, a polymorph thereof, a stereoisomer thereof, a geometric isomer thereof, a tautomer thereof, a mixture of tautomers thereof, an enantiomer thereof, a diastereomer thereof, a racemate thereof, a mixture of stereoisomers thereof, an isotope thereof (e.g., tritium, deuterium), or a combination thereof.

Compounds described herein may contain one or more asymmetric centers and may thus give rise to enantiomers, diastereomers, and other stereoisomeric forms. Each chiral center may be defined, in terms of absolute stereochemistry, as (R)- or (S)-. This technology is meant to include all such possible isomers, as well as mixtures thereof, including racemic and optically pure forms. Optically active (R)- and (S)-, (−)- and (+)-, or (D)- and (L)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both (E)- and (Z)-geometric isomers. Likewise, all tautomeric forms are also intended to be included.

Compounds described herein contain hydrazone bonds. All possible cis/trans/s-cis/s-trans isomers of the hydrazone bonds are intended to be encompassed within the scope of the present application. This technology is meant to include all such possible isomers, as well as mixtures thereof.

Compounds described herein may also contain isotopes at one or more of the atoms that constitute such compounds. For example, the compounds may be radiolabeled with radioactive isotopes, such as for example tritium (³H), iodine-125 (¹²I), carbon-14 (¹⁴C), carbon-11 (¹¹C), or fluorine-18 (¹⁸F). All isotopic variations of the compounds of the present application, whether radioactive or not, are intended to be encompassed within the scope of the present application.

According to the present application, in the nanocage of Formula (I), p can be 1, 2, 3, 4, or 5. In one embodiment, p is 1 to 3.

In one embodiment, at least one A in the nanocage of Formula (I) is different from the other A.

In another embodiment, all R in the nanocage of Formula (I) are the same.

In another embodiment, all R in the nanocage of Formula (I) are different.

In yet another embodiment, at least one R in the nanocage of Formula (I) is different from the other R.

In a further embodiment, the nanocage is Td-symmetric.

The height of the nanocage can be determined with standard molecular modeling tools with the “measure distance” application programming interfaces (APIs) (Humphrey et al., “VMD—Visual Molecular Dynamics,” J. Molec. Graphics. 14:33-38 (1996); Hanwell et al., “Avogadro: An Advanceed Semantic Chemical Editor, Visualization, and Analysis Platform,” J. Cheminform. 4:17 (2012); Guex et al., “SWISS-MODEL and the Swiss-PDB Viewer: An Environment for Comparative Protein Modeling,” Electrophoresis 18:2714-2723 (1997); Pirhadi et al., “Open Source Molecular Modeling,” J. Mol. Graphics Modell. 69:127-143 (2016), which are hereby incorporated by reference in their entirety).

According to the present application, the nanocage has a height (an internal height) from 15 Å to 50 Å. Preferably, the nanocage has a height from 15 Å to 49 Å, from 15 Å to 48 Å, from 15 Å to 47 Å, from 15 Å to 46 Å, from 15 Å to 45 Å, from 16 Å to 45 Å, from 17 Å to 44 Å, from 18 Å to 43 Å, from 19 Å to 42 Å, from 20 Å to 41 Å, from 20 Å to 40 Å, from 20 Å to 35 Å, from 20 Å to 30 Å, from 20 Å to 29 Å, from 20 Å to 28 Å, from 20 Å to 27 Å, from 20 Å to 26 Å, from 20 Å to 25 Å. More preferably, the nanocage has a height (an internal height) from 20 Å to 40 Å, 25 Å to 40 Å, from 25 Å to 38 Å, from 30 Å to 38 Å, from 32 Å to 38 Å, from 32 Å to 36 Å, 20 Å to 30 Å, 21 Å to 30 Å, from 22 Å to 30 Å, from 23 Å to 30 Å, from 24 Å to 30 Å, from 25 Å to 30 Å, from 26 Å to 30 Å, from 27 Å to 30 Å, from 28 Å to 30 Å, from 29 Å to 30 Å, 20 Å to 29 Å, from 20 Å to 28 Å, from 20 Å to 27 Å, from 20 Å to 26 Å, 20 Å to 25 Å, from 20 Å to 24 Å, from 20 Å to 23 Å, from 20 Å to 22 Å, from 20 Å to 21 Å, from 21 Å to 29 Å, from 22 Å to 28 Å, from 23 Å to 27 Å, from 24 Å to 26 Å.

As used herein the term “internal height of the nanocage” or “height of the nanocage” is the height of the nanocage itself (triglyme groups on the outside of the tetrahedron (cage) are not considered for the height determination). The height (an internal height) can be measured using molecular modeling software such as Maestro (Schrödinger Release 2023-2: Maestro, Schrödinger, LLC, New York, NY, 2023) or similar software tools using standard techniques known to those skilled in the art.

According to the present application, the nanocage has a solvodynamic radius of from about 1 to about 10 nm, from about 1 to about 8 nm, from about 1 to about 6 nm, from about 1.5 to about 5 nm, from about 2 to about 5 nm, from about 2.5 to about 5 nm, or from about 2.5 to about 4 nm. For example, the nanocage has a solvodynamic radius of about 1.0 nm, about 1.1 nm, about 1.2 nm, about 1.3 nm, about 1.4 nm, about 1.5 nm, about 1.6 nm, about 1.7 nm, about 1.8 nm, 1.9 nm, 2.0 nm, about 2.1 nm, about 2.2 nm, about 2.3 nm, about 2.4 nm, about 2.5 nm, about 2.6 nm, about 2.7 nm, about 2.8 nm, 2.9 nm, 3.0 nm, about 3.1 nm, about 3.2 nm, about 3.3 nm, about 3.4 nm, about 3.5 nm, about 3.6 nm, about 3.7 nm, about 3.8 nm, 3.9 nm, 4.0 nm, about 4.1 nm, about 4.2 nm, about 4.3 nm, about 4.4 nm, about 4.5 nm, about 4.6 nm, about 4.7 nm, about 4.8 nm, 4.9 nm, or 5.0 nm.

As used herein the term “solvodynamic radius” refers to the Stokes-Einstein radius of a solute. This radius defines a sphere, which includes the average amount of solvent diffusing with the solute. The solvodynamic radius can be measured using diffusion-ordered spectroscopy (DOSY) NMR using the following method. First, average diffusion coefficients for the nuclei (for example, the ¹H nuclei of the nanocage) molecule are obtained from the DOSY NMR spectra, using standard methods implemented in NMR analysis software such as TopSpin (Donaldson et al., “A Computational Group Theoretic Symmetry Reduction Package for the SPIN Model Checker,” In Proceedings of the 11th International Conference on Algebraic Methodology and Software Technology (AMAST'06), Lecture Notes in Computer Science 4019, p. 374-380, Springer (2006), which is hereby incorporated by reference in its entirety). Next, by using Stokes-Einstein Equation, which assumes a spherical model, these diffusion coefficients can be translated into approximate solvodynamic radius by solving the Stokes Einstein equation for the solvodynamic radius.

According to the present application, the nanocage has a cavity volume of from about 5 to about 30 nm³, from about 10 to about 25 nm³, from about 15 to about 25 nm³, or from about 15 to about 20 nm³. For example, the nanocage has a cavity volume of about 10.0 nm³, about 11 nm³, about 12 nm³, about 13 nm³, about 14 nm³, about 15 nm³, about 16 nm³, about 17 nm³, about 18 nm³, about 19 nm³, about 20 nm³, about 21 nm³, about 22 nm³, about 23 nm³, about 24 nm³, about 25 nm³, about 26 nm³, about 27 nm³, about 28 nm³, about 29 nm³, or about 30 nm³.

As used herein the term “cavity volume” refers to the volume of the cavity of the tetrahedron (triglyme groups on the outside of the tetrahedron (cage) are not considered for the determination of cavity volume). The cavity volume can be measured using computational chemistry with software such as Maestro (Schrödinger Release 2023-2: Maestro, Schrödinger, LLC, New York, NY, 2023), MoloVol (Maglic et al., “MoloVol: An Easy-To-Use Program For Analyzing Cavities, Volumes and Surface Areas of Chemical Structures,” J. Appl. Crystallogr. 55(Pt 4):1033-1044 (2022), which is hereby incorporated by reference in its entirety), or similar software tools.

One embodiment relates to the nanocage of Formula (I), wherein R¹, R², and R³ are Me, R⁴ is —OMe, R⁵ is —O(CH₂)₄—(OCH₂CH₂)₃—OMe, and R⁶ is —C₆H₁₃.

In one embodiment, the nanocage of Formula (I) is stable in water.

In another embodiment, the nanocage of Formula (I) is stable in water at elevated temperatures. For example, the nanocage of Formula (I) is stable in water at 30° C., 40° C., 50° C., 60° C., 70° C., 80° C., 90° C., or 100° C.

The general scheme for the synthesis of nanocages of the present application is shown in Scheme 1.

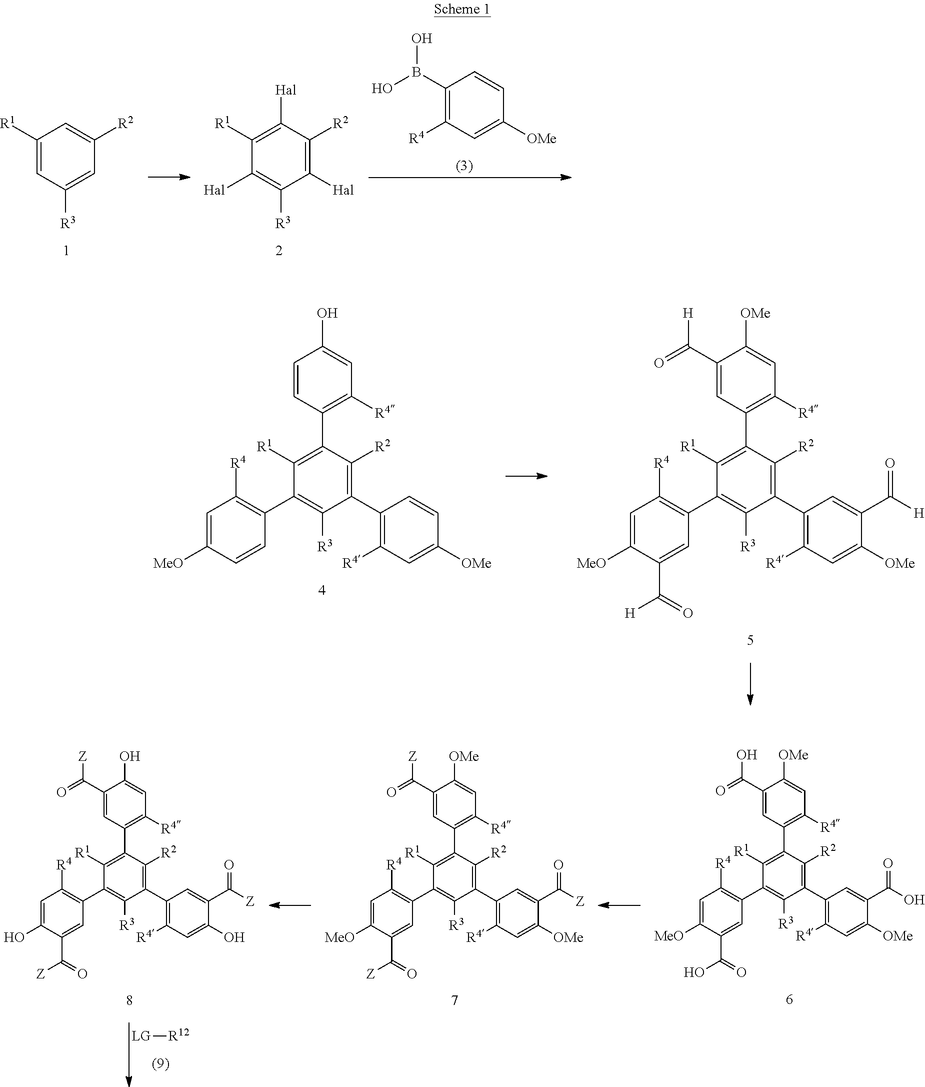

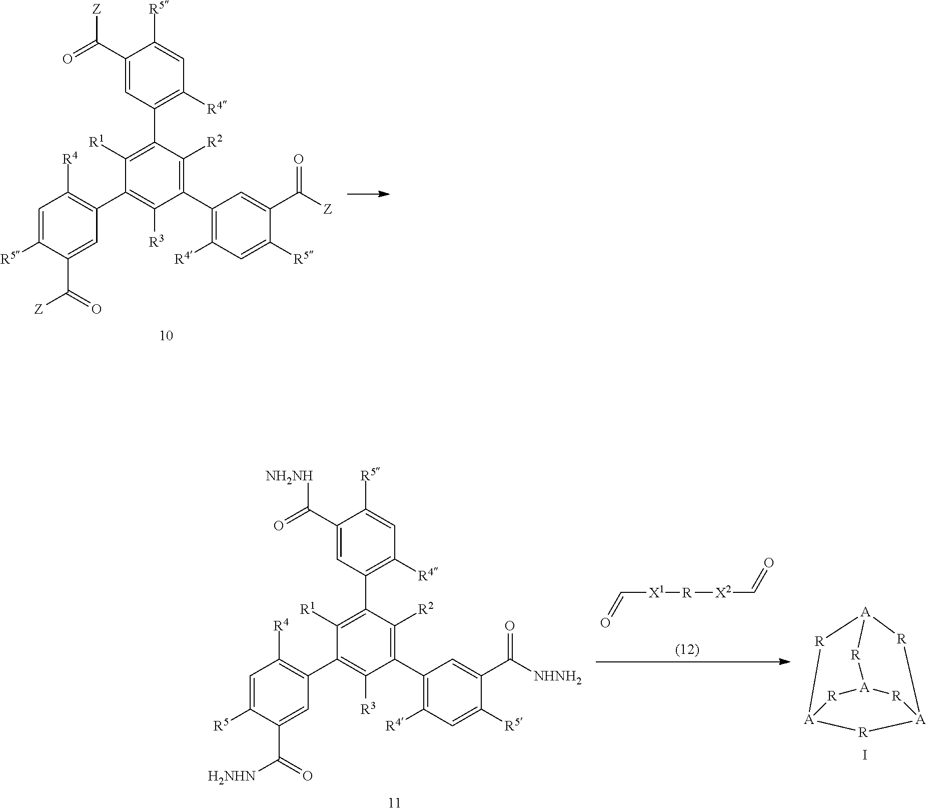

each A is independently selected and has the formula

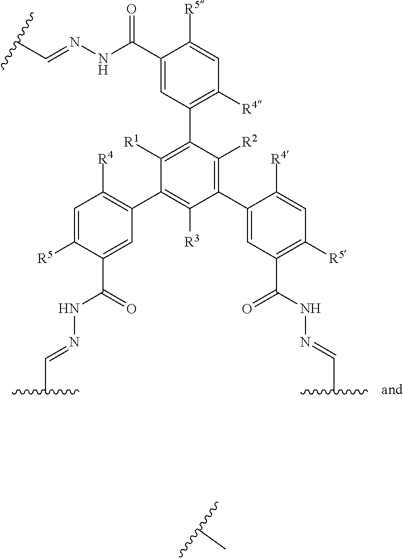

is the point of attachment of A to R.

Halogenation of compound 1 leads to formation of compound 2. Any suitable halogenating agent can be used, for example, I₂, N-iodosuccinimide (NIS), or Br₂. The reaction can be carried out in a variety of solvents, for example in water, methylene chloride (CH₂Cl₂), tetrahydrofuran (THF), dimethylformamide (DMF), or other such solvents or in the mixture of such solvents. This reaction can be carried out at room temperature or at elevated temperatures. Coupling of compound 2 with boronic acid 3 leads to formation of compound 4. The reaction can be carried out in a variety of solvents, for example in water, methylene chloride (CH₂Cl₂), tetrahydrofuran (THF), dimethylformamide (DMF), or other such solvents or in the mixture of such solvents. This reaction can be carried out at room temperature or at elevated temperatures. Formylation of compound 4 leads to formation of trialdehyde 5. Any suitable formylation reagent can be used, for example, DMF/POCl₃ and paraformaldehyde/BF₃. The reaction can be carried out in a variety of solvents, for example in methylene chloride (CH₂Cl₂), tetrahydrofuran (THF), dimethylformamide (DMF), or other such solvents or in the mixture of such solvents.

Oxidation of trialdehyde 5 leads to formation of acid 6. Suitable oxidizing agents that can be used include NaClO₂, oxone, H₅IO₆, H₂O₂, O₂, sodium perborate, or KMnO₄. The reaction can be carried out in a variety of solvents, for example in methylene chloride (CH₂Cl₂), tetrahydrofuran (THF), dimethylformamide (DMF), or other such solvents or in the mixture of such solvents. Esterification of compound 6 leads to formation of ester 7. Hydroxyl groups in ester 7 can be alkylated to form compound 8. Compound 8 is reacted with compound 9 to form compound 10. The reaction can be carried out in a variety of solvents, for example in methylene chloride (CH₂Cl₂), tetrahydrofuran (THF), dimethylformamide (DMF), or other such solvents or in the mixture of such solvents. Reaction of compound 10 with the hydrozine source leads to formation of compound 11. Suitable hydrozine sources that can be used include hydrazine, hydrazine hydrate, and hydrazine salts (for example hydrazine monohydrochloride). This reaction can be carried out at room temperature or at elevated temperatures. Reaction of compound 11 with dialdehyde 12 affords nanocage of Formula (I). The reaction can be carried out in a variety of solvents, for example in methylene chloride (CH₂Cl₂), tetrahydrofuran (THF), dimethylformamide (DMF), or other such solvents or in the mixture of such solvents.

During the reaction process described in Scheme 1, the non-participating carboxylic acids can be protected by a suitable protecting group (PG) which can be selectively removed at a later time if desired. A detailed description of these groups and their selection and chemistry is contained in “The Peptides, Vol. 3”, Gross and Meinenhofer, Eds., Academic Press, New York, 1981, which is hereby incorporated by reference in its entirety.

Another aspect of the present application relates to a process for preparation of a nanocage of Formula (I):

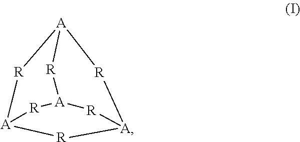

each A is independently selected and has the formula

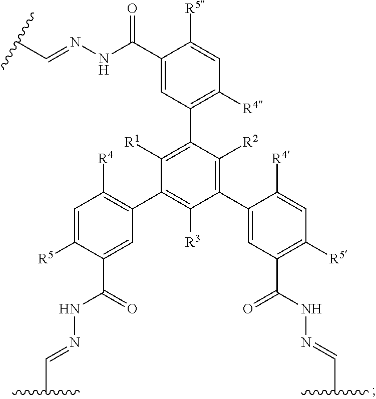

is the point of attachment of A to R;
each R is independently selected and has the formula

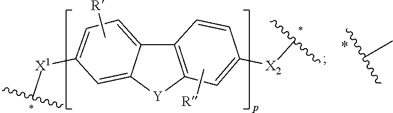

indicates the point of attachment of R to A;
R′ is H or C₁₋₂₀ alkyl;
R″ is H or C₁₋₂₀ alkyl;
R¹, R², and R³ are each independently selected from the group consisting of H, halogen, —OH, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁴, R^{4′}, and R^{4″} are each independently selected from the group consisting of H, halogen, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OH, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀-alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁵, R^{5′}, and R^{5″} are each independently selected from the group consisting of H, halogen, —OH, —O(CH₂)ₙ—(OCH₂CH₂)ₘ—OC₁₋₆ alkyl, —O(CH₂)ₙ—(OCH₂CH₂)ₘ—OH, (OCH₂CH₂)ₘ—OC₁₋₆ alkyl, (OCH₂CH₂)ₘ—OH, aryl, heteroaryl, heterocyclyl, —OC₁₋₂₀ alkyl, —O-perfluorinated C₁₋₂₀alkyl, —Oaryl, and —NR¹⁰R¹¹;
R⁶ and R^{6′} are each independently selected at each occurrence from the group consisting of H, C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl, wherein C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl can be substituted from 1 to 3 times with a substituent selected from the group consisting of —OC₁₋₂₀ alkyl, —OC₂₋₂₀ alkenyl, OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀-alkynyl, —CONH perfluorinated C₁₋₂₀ alkyl, —CONH-aryl, —CONH-heteroaryl, and CONH-heterocyclyl;
R¹⁰ and R¹¹ are each independently selected from the group consisting of H, C₁₋₆ alkyl, C₂₋₆ alkenyl, C₂₋₆ alkynyl, aryl, heteroarylalkyl, heterocyclylalkyl, and arylalkyl, wherein each C₁₋₆ alkyl, C₂₋₆ alkenyl, C₂₋₆ alkynyl, aryl, heteroarylalkyl, heterocyclylalkyl, and arylalkyl can be optionally substituted from 1 to 3 times with a substituent selected from the group consisting of H, C₁₋₆ alkyl, heteroarylalkyl, heterocyclylalkyl, and arylalkyl;
X¹ and X² are absent or are independently selected from the group consisting of C₁₋₆ alkylene, C₃₋₈ cycloalkylene, and arylene, wherein C₁₋₆ alkylene, C₃₋₈ cycloalkylene, and arylene can be optionally substituted from 1 to 3 times with H or C₁₋₆ alkyl;
Y is independently selected from the group consisting of —N(R⁶)—, —S—, —O—, —C(R⁶)(R^{6′})—, and —P(R⁶)—;
p is 1 to 5;
n is 1 to 10; and
m is 1 to 50, said process comprising:
providing one or more compounds of Formula (II) having the structure:

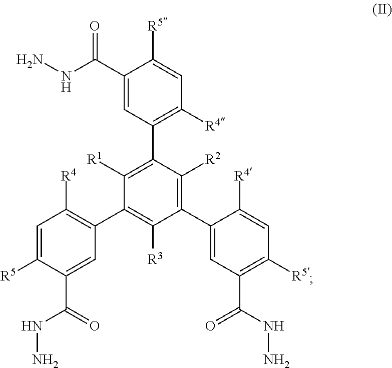

forming the nanocage of Formula (I) from the one or more compounds of Formula (II).

According to the present application, in the nanocage of Formula (I), p can be 1, 2, 3, 4, or 5. In one embodiment, p is 1 to 3.

One embodiment of the present invention further comprises reacting the one or more compounds of Formula (II) with one or more compounds of Formula (III):

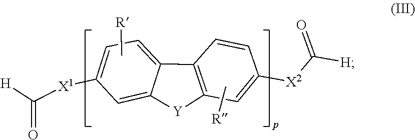

to produce the nanocage of Formula (I).

In one embodiment, the compound of Formula (III) has the following structure:

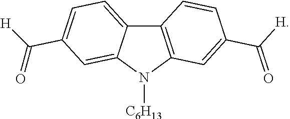

Another embodiment of the present invention further comprises providing one or more compounds of Formula (IV) having the structure:

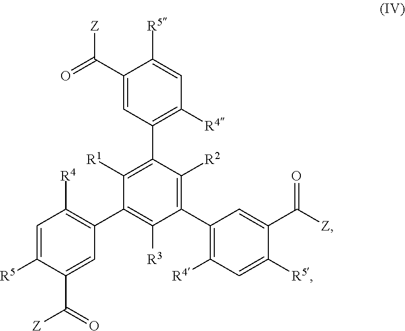

wherein
Z is —O—C₁₋₆ alkyl; and
forming the one or more compounds of Formula (II) from the one or more compounds of Formula (IV).

In accordance with one embodiment, forming the one or more compounds of Formula (II) may comprise reacting the one or more compounds of Formula (IV) with a hydrazine source to produce the one or more compounds of Formula (II).

The hydrazine source that can be used according to the present application includes, but is not limited to, hydrazine, hydrazine hydrate, or hydrazine salts (for example hydrazine monohydrochloride).

Another embodiment of the present invention further comprises providing one or more compounds of Formula (V) having the structure:

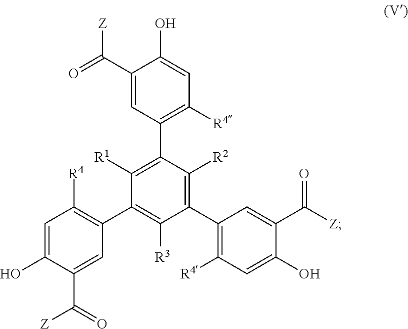

forming the one or more compounds of Formula (IV) from the one or more compounds of Formula (V).

In accordance with one embodiment, forming the one or more compounds of Formula (IV) may comprise reacting the one or more compounds of Formula (V) with one or more compounds of Formula (VI):

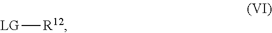

wherein LG is a suitable leaving group; and
R¹² is selected from a group consisting of (CH₂)ₙ—(OCH₂CH₂)ₘ—OC₁₋₆ alkyl, —(CH₂)ₙ—(OCH₂CH₂)ₘ—OH, CH₂CH₂—(OCH₂CH₂)ₘ-1-OC₁₋₆ alkyl, CH₂CH₂—(OCH₂CH₂)ₘ₋₁—OH, C₁₋₂₀ alkyl, -perfluorinated C₁₋₂₀ alkyl, and -aryl;
to produce the one or more compounds of Formula (IV).

In one embodiment, the one or more compounds of Formula (VI) has the formula:

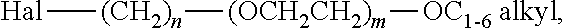

wherein Hal is Cl or Br.

Another embodiment of the present invention further comprises providing one or more compounds of Formula (VII) having the structure:

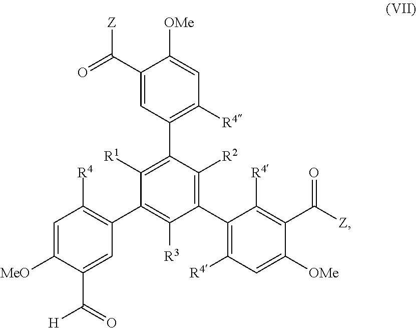

forming the one or more compounds of Formula (V) from the one or more compounds of Formula (VII).

In accordance with one embodiment, forming the one or more compounds of Formula (V) may comprise reacting the compound of Formula (VII) with a compound of Formula (VIII):

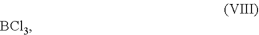

to produce the one or more compounds of Formula (V).

Another embodiment of the present invention further comprises providing one or more compounds of Formula (IX) having the structure:

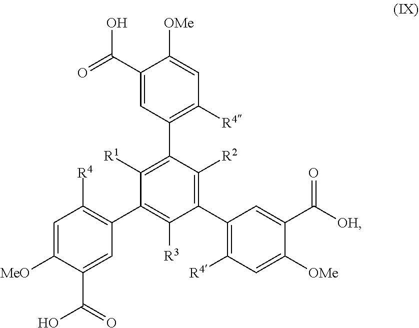

esterifying the one or more compounds of Formula (IX) to produce the one or more compounds of Formula (VII).

In accordance with one embodiment, esterifying the one or more compounds of Formula (IX) may comprise reacting the one or more compounds of Formula (IX) with MeOH to produce the one or more compounds of Formula (VII).

Another embodiment of the present invention further comprises providing one or more compounds of Formula (X) having the structure:

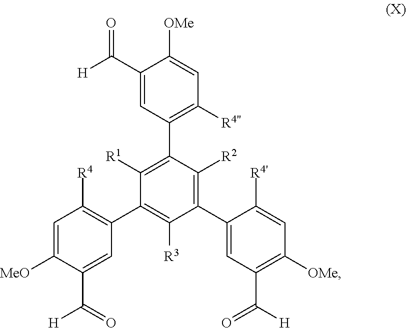

forming the one or more compounds of Formula (IX) from the one or more compounds of Formula (X).

In accordance with one embodiment, forming the one or more compounds of Formula (IX) may comprise reacting the compound of Formula (X) with an oxidizing agent to produce the one or more compounds of Formula (IX). Suitable oxidizing agents include, but are not limited to, NaClO₂, oxone (potassium peroxymonosulfate), H₅IO₆, H₂O₂, O₂, sodium perborate, and KMnO₄.

Another embodiment of the present invention further comprises providing one or more compounds of Formula (XI) having the structure:

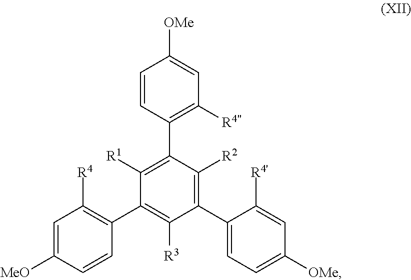

forming the one or more compounds of Formula (X) from the one or more compounds of Formula (XI).

In accordance with one embodiment, forming the one or more compounds of Formula (X) may comprise reacting the one or more compounds of Formula (XI) with a formylating agent to produce the one or more compounds of Formula (X). Suitable formylating agent include, but are not limited to, DMF/POCl₃ and paraformaldehyde/BF₃.

Another embodiment of the present invention further comprises providing one or more compounds of Formula (XII) having the structure:

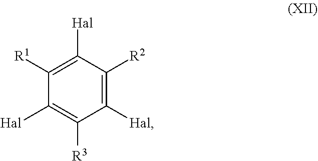

wherein Hal is halogen; and
forming the one or more compounds of Formula (XI) from the one or more compounds of Formula (XII)

In accordance with one embodiment, forming the one or more compounds of Formula (XI) may comprise reacting the one or more compounds of Formula (XII) with one or more compounds of Formula (XIII):

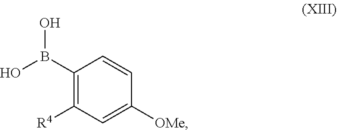

to produce the one or more compounds of Formula (XI).

Another embodiment of the present invention further comprises providing one or more compounds of Formula (XIV) having the structure:

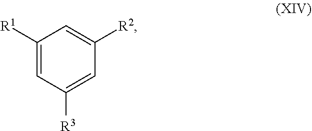

forming the one or more compounds of Formula (XII) from the one or more compounds of Formula (XIV).

In accordance with one embodiment, forming the one or more compounds of Formula (XII) may comprise reacting the one or more compounds of Formula (XIV) with halogenating agent to produce the one or more compounds of Formula (XII). Suitable halogenating agents that can be used include, but are not limited to, I₂, N-iodosuccinimide (NIS), and Br₂.

Another aspect of the present application relates to a process for preparation of a nanocage of Formula (I′):

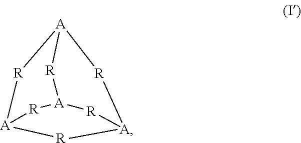

wherein
each A is independently selected and has the formula

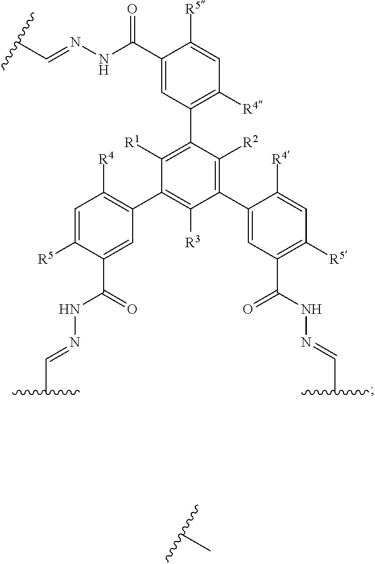

is the point of attachment of A to R;
each R is independently selected and has the formula

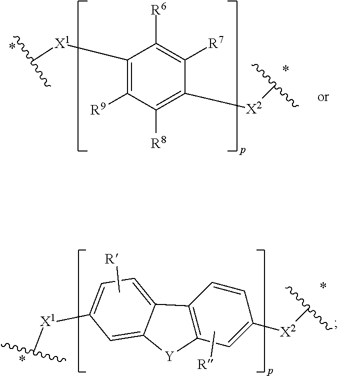

indicates the point of attachment of R to A;
R′ is H or C₁₋₂₀ alkyl;
R″ is H or C₁₋₂₀ alkyl;
R¹, R², and R³ are each independently selected from the group consisting of H, halogen, —OH, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀-alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;

R⁴, R^{4′}, and R^{4″} are each independently selected from the group consisting of H, halogen, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OH, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀-alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁵, R^{5′}, and R^{5″} are each independently selected from the group consisting of H, halogen, —OH, —O(CH₂)ₙ—(OCH₂CH₂)ₘ—OC₁₋₆ alkyl, —O(CH₂)ₙ—(OCH₂CH₂)ₘ—OH, (OCH₂CH₂)ₘ—OC₁₋₆ alkyl, (OCH₂CH₂)ₘ—OH, aryl, heteroaryl, heterocyclyl, —OC₁₋₂₀ alkyl, —O-perfluorinated C₁₋₂₀ alkyl, —Oaryl, and —NR¹⁰R¹¹;
R^{6′} and R^{6″} are each independently selected at each occurrence from the group consisting of H, C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl, wherein C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl can be substituted from 1 to 3 times with a substituent selected from the group consisting of —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀-alkynyl, —CONH perfluorinated C₁₋₂₀ alkyl, —CONH-aryl, —CONH-heteroaryl, and CONH-heterocyclyl;
R⁶, R⁷, R⁸, and R⁹ are each independently selected at each occurrence from the group consisting of H, halogen, C₁₋₂₀ alkyl, C₂₋₂₀ alkenyl, C₂₋₂₀ alkynyl, perfluorinated C₁₋₂₀ alkyl, aryl, heteroaryl, heterocyclyl, OH, —OC₁₋₂₀ alkyl, —OC₂₋₂₀ alkenyl, —O-perfluorinated C₁₋₂₀ alkyl, —OC₂₋₂₀ alkynyl, aryl, heteroaryl, heterocyclyl, and —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, CONH perfluorinated C₁₋₂₀ alkyl, —CONH-aryl, —CONH-heteroaryl, and —CONH— heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl can be optionally substituted from 1 to 3 times with a substituent selected from the group consisting of H, halogen, OH, C₁₋₆ alkyl, aryl, and arylalkyl;
R¹⁰ and R¹¹ are each independently selected from the group consisting of H, C₁₋₆ alkyl, C₂₋₆ alkenyl, C₂₋₆ alkynyl, aryl, heteroarylalkyl, heterocyclylalkyl, and arylalkyl, wherein each C₁₋₆ alkyl, C₂₋₆ alkenyl, C₂₋₆ alkynyl, aryl, heteroarylalkyl, heterocyclylalkyl, and arylalkyl can be optionally substituted from 1 to 3 times with a substituent selected from the group consisting of H, C₁₋₆ alkyl, heteroarylalkyl, heterocyclylalkyl, and arylalkyl;
X¹ and X² are absent or are independently selected from the group consisting of C₁₋₆ alkylene, C₃₋₈ cycloalkylene, and arylene, wherein C₁₋₆ alkylene, C₃₋₈ cycloalkylene, and arylene can be optionally substituted from 1 to 3 times with H or C₁₋₆ alkyl;
Y is independently selected from the group consisting of —N(R^{6′})—, —S—, —O—, —C(R^{6′})(R^{6″})—, and —P(R⁶)—;
p is 1 to 5;
n is 1 to 10; and
m is 1 to 50, said process comprising:
providing one or more compounds of Formula (V′) having the structure:

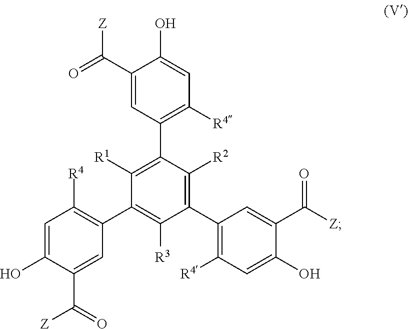

wherein Z is —O—C₁₋₆ alkyl; and
forming the nanocage of Formula (I′) from the one or more compounds of Formula (V′).

According to the present application, in the nanocage of Formula (I′), p can be 1, 2, 3, 4, or 5. In one embodiment, p is 1 to 3.

In accordance with one embodiment, forming the nanocage of Formula (I′) may comprise reacting the one or more compounds of Formula (V′) with one or more compounds of Formula (VI′):

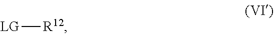

wherein LG is a suitable leaving group; and
R¹² is selected from a group consisting of (CH₂)ₙ—(OCH₂CH₂)ₘ—OC₁₋₆ alkyl, —(CH₂)ₙ—(OCH₂CH₂)ₘ—OH, CH₂CH₂—(OCH₂CH₂)ₘ₋₁—OC₁₋₆ alkyl, CH₂CH₂—(OCH₂CH₂)ₘ₋₁—OH, C₁₋₂₀ alkyl, -perfluorinated C₁₋₂₀ alkyl, and -aryl;
to produce the one or more compounds of Formula (IV′):

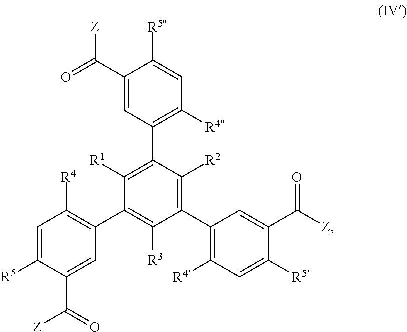

wherein Z is —O—C₁₋₆ alkyl;
reacting the one or more compounds of Formula (IV′) with a hydrazine source to produce the one or more compounds of Formula (II′):

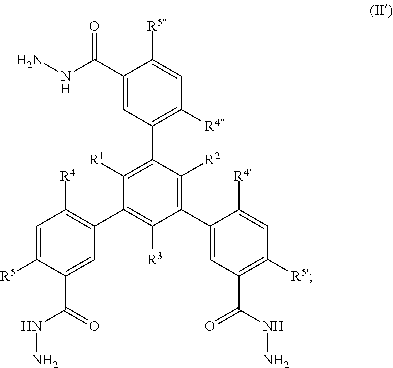

reacting one or more compounds of Formula (II′) with one or more compounds of Formula (III′A) or Formula (III′B):

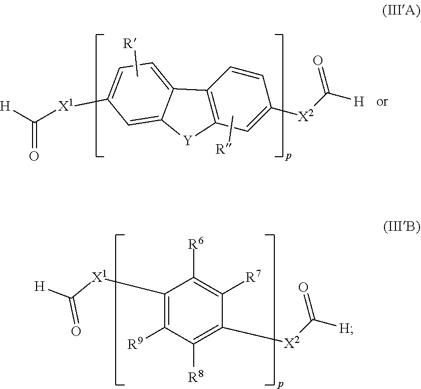

to produce the nanocage of Formula (I′).

In one embodiment, the compound of Formula (III′A) has the following structure:

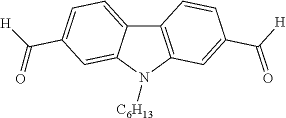

In another embodiment, the compound of Formula (III′B) has the following structure:

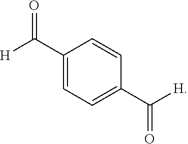

In another embodiment, the compound of Formula (III′B) has the following structure:

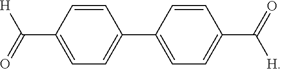

In another embodiment, the compound of Formula (III′B) has the following structure:

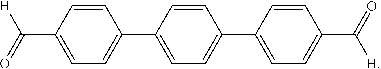

In another embodiment, the compound of Formula (III′B) has the following structure:

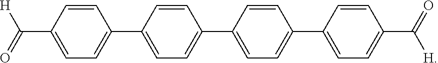

In another embodiment, the one or more compounds of Formula (VI′) has the formula:

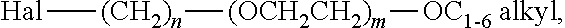

wherein Hal is Cl or Br.

Another embodiment of the present invention further comprises providing one or more compounds of Formula (VII′) having the structure:

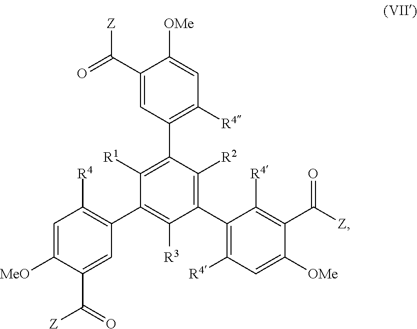

and
forming the one or more compounds of Formula (V′) from the one or more compounds of Formula (VII′).

In accordance with one embodiment, forming the one or more compounds of Formula (V′) may comprise reacting the compound of Formula (VII′) with a compound of Formula (VIII′):

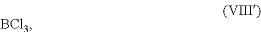

to produce the one or more compounds of Formula (V′).

Another embodiment of the present invention further comprises providing one or more compounds of Formula (IX′) having the structure:

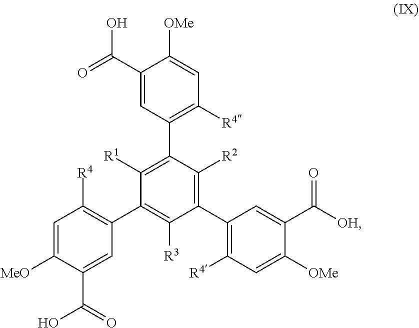

and
esterifying the one or more compounds of Formula (IX′) to produce the one or more compounds of Formula (VII′).

In accordance with one embodiment, esterifying the one or more compounds of Formula (IX′) may comprise reacting the one or more compounds of Formula (IX′) with MeOH to produce the one or more compounds of Formula (VII′)

Another embodiment of the present invention further comprises providing one or more compounds of Formula (X′) having the structure:

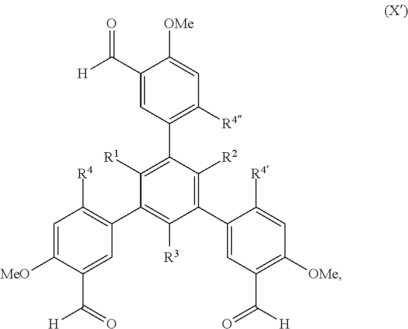

and
forming the one or more compounds of Formula (IX′) from the one or more compounds of Formula (X′).

In accordance with one embodiment, forming the one or more compounds of Formula (IX′) may comprise reacting the compound of Formula (X′) with an oxidizing agent to produce the one or more compounds of Formula (IX′). Suitable oxidizing agents include, but are not limited to, NaClO₂, oxone (potassium peroxymonosulfate), H₅IO₆, H₂O₂, O₂, sodium perborate, and KMnO₄.

Another embodiment of the present invention further comprises providing one or more compounds of Formula (XI′) having the structure:

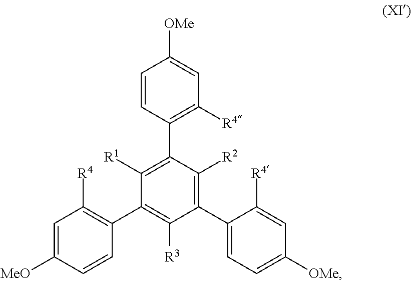

and
forming the one or more compounds of Formula (X′) from the one or more compounds of Formula (XI′).

In accordance with one embodiment, forming the one or more compounds of Formula (X′) may comprise reacting the one or more compounds of Formula (XI′) with a formylating agent to produce the one or more compounds of Formula (X′). Suitable formylating agents include, but are not limited to, DMF/POCl₃ and paraformaldehyde/BF₃.

Another embodiment of the present invention further comprises providing one or more compounds of Formula (XII′) having the structure:

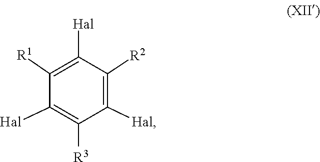

wherein
Hal is halogen; and
forming the one or more compounds of Formula (XI′) from the one or more compounds of Formula (XII′).

In accordance with one embodiment, forming the one or more compounds of Formula (XI′) may comprise reacting the one or more compounds of Formula (XII′) with one or more compounds of Formula (XIII′):

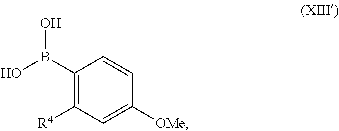

to produce the one or more compounds of Formula (XI′).

Another embodiment of the present invention further comprises providing one or more compounds of Formula (XIV′) having the structure:

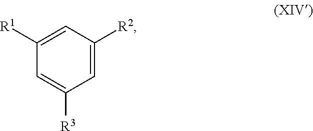

and
forming the one or more compounds of Formula (XII′) from the one or more compounds of Formula (XIV′)

In accordance with one embodiment, forming the one or more compounds of Formula (XII′) may comprise reacting the one or more compounds of Formula (XIV′) with halogenating agent to produce the one or more compounds of Formula (XII′). Suitable halogenating agents include, but are not limited to, I₂, N-iodosuccinimide (NIS), and Br₂.

Another aspect of the present application relates to a method for detecting an analyte in a fluid. This method includes:
providing a sensor comprising a nanocage of formula (I):

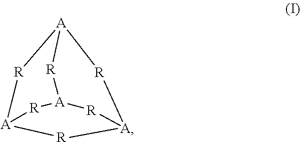

wherein
each A is independently selected and has the formula

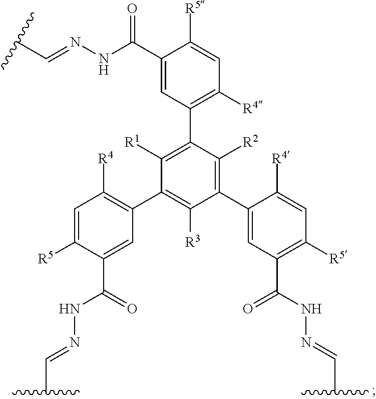

is the point of attachment of A to R;
each R is independently selected and has the formula

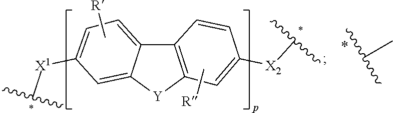

indicates the point of attachment of R to A;
R′ is H or C₁₋₂₀ alkyl;
R″ is H or C₁₋₂₀ alkyl;
R¹, R², and R³ are each independently selected from the group consisting of H, halogen, —OH, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁴, R^{4′}, and R^{4″} are each independently selected from the group consisting of H, halogen, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OH, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀-alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁵, R^{5′}, and R^{5″} are each independently selected from the group consisting of H, halogen, OH, —O(CH₂)ₙ—(OCH₂CH₂)ₘ—OC₁₋₆ alkyl, —O(CH₂)ₙ—(OCH₂CH₂)ₘ—OH, (OCH₂CH₂)ₘ—OC₁₋₆ alkyl, (OCH₂CH₂)ₘ—OH, aryl, heteroaryl, heterocyclyl, —OC₁₋₂₀ alkyl, —O-perfluorinated C₁₋₂₀alkyl, —Oaryl, and —NR¹⁰R¹¹;
R⁶ and R^{6′} are each independently selected at each occurrence from the group consisting of H, C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl, wherein C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl can be substituted from 1 to 3 times with a substituent selected from the group consisting of —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl;
R¹⁰ and R¹¹ are each independently selected from the group consisting of H, C₁₋₆ alkyl, C₂₋₆ alkenyl, C₂₋₆ alkynyl, aryl, heteroarylalkyl, heterocyclylalkyl, and arylalkyl, wherein each C₁₋₆ alkyl, C₂₋₆ alkenyl, C₂₋₆ alkynyl, aryl, heteroarylalkyl, heterocyclylalkyl, and arylalkyl can be optionally substituted from 1 to 3 times with a substituent selected from the group consisting of H, C₁₋₆ alkyl, heteroarylalkyl, heterocyclylalkyl, and arylalkyl;
X¹ and X² are absent or are independently selected from the group consisting of C₁₋₆ alkylene, C₃₋₈ cycloalkylene, and arylene, wherein C₁₋₆ alkylene, C₃₋₈ cycloalkylene, and arylene can be optionally substituted from 1 to 3 times with H or C₁₋₆ alkyl;
Y is independently selected from the group consisting of —N(R⁶)—, —S—, —O—, —C(R⁶)(R^{6′})—, and —P(R⁶)—;
p is 1 to 5;
n is 1 to 10; and
m is 1 to 50;
providing a fluid containing an analyte; and
contacting a fluid containing the analyte with the sensor to capture the analyte in the nanocage and detect the analyte in the fluid.

According to the present application, in the nanocage of Formula (I), p can be 1, 2, 3, 4, or 5. In one embodiment, p is 1 to 3.

In one embodiment, the method further comprises providing a signal generator operatively associated with said sensor, said method further comprising:
producing a signal with the signal generator when said analyte is captured by said sensor.

Suitable analytes that can be detected in accordance with the present application include, but are not limited to, polyvinylpyrrolidone (PVP), poly(isobutylene-alt-n-octyl maleimide) (POI), picrocrocin, curcumin, and components of chinese tea.

According to the present application, the sensor further comprises a substrate having a surface with a layer of the nanocage of Formula (I) covering at least 1% of the surface. Preferably, the layer of the nanocage of Formula (I) covers at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25% of the substrate's surface.

According to the present application, the suitable substrates include paper, plastics (e.g., photoresist materials, acrylic polymers, carbonate polymers, etc.), glass, silicon based materials (e.g., silicon, silicon dioxide, silicon nitride, etc.) and metals.

In one embodiment, the substrate is a paper strip.

In another embodiment, the sensor further comprises a fluorescent polymer.

Suitable fluorescent polymers that can be used according to the present application include, but are not limited to, dye-functionalized glycol polymers (e.g. polyethylene glycol, polypropylene glycol, polybutylene glycol, etc.), poly-alkyl-imides (including polyisobutylene-alt-n-octyl imide), and polyacrylates.

In one embodiment, a fluorescent dye is attached to a nanocage of Formula (I). Suitable fluorescent dyes that can be used include, but are not limited to, Cy3-azide, Cy5-azide, Cy3-alkyne, Cy5-alkyne, Azide-fluor 488, Azide-fluor 545, Azide cyanine dye 728, DBCO-Cy3, Azide MegaStokes dye 673.

In another embodiment, a fluorescent dye is attached to an alkylene-containing nanocage of Formula (I) using click chemistry.

In yet another embodiment, the sensor also contains polymers containing fluorescent dies. Suitable polymers that can be used according to the present application include glycol polymers, poly-alkyl-imides, and polyacrylates.

In one embodiment, the method for detecting an analyte in a fluid can provide detection in real time.

In another embodiment, detection of an analyte in a fluid by the sensor will result in a change in color that could be directly observed by a naked eye. Alternatively, the change in color can be observed by applying a light source.

Suitable light sources that can be used in accordance to the present invention include, but are not limited to, light emitting diodes (LED), flash lamps, cold-cathode fluorescent lamps, and electroluminescent lamps. The illumination may be multiplexed and/or collimated. In some cases, the illumination may be pulsed to reduce any background interference. Further, illumination may be continuous or may combine continuous wave (CW) and pulsed illumination where multiple illumination beams are multiplexed (e.g., a pulsed beam is multiplexed with a CW beam), permitting signal discrimination between a signal induced by the CW source and a signal induced by the pulsed source.

In one embodiment, detection of an analyte in a fluid by the sensor will result in a change in color that could be observed by using yellow LED lamp.

According to the present application, qualitative, quantitative, or semi-quantitative determination of the presence or concentration of an analyte may be achieved.

In another embodiment, the color change is concentration-dependent. The higher concentration of the analyte, the bigger is the change in color.

In a further embodiment, the sensor is a nanosensor.

In yet another embodiment, the sensor tests the quality of saffron.

During the manufacture of sensor, a sensor material comprising the nanocage of Formula (I) is placed on the substrate. This sensor material may be deposited, coated, or otherwise applied on the substrate.

Different sensing elements can be deposited in different areas of the substrate to form sensing arrays. Analyte samples of different quality then lead to specific fingerprints of color patterns with the sensing arrays under blue, yellow, green, or red, LED illumination. The color patterns can be recorded with a smartphone camera and analyzed (see, for an example, Kim et al., “Prediction of Key Aroma Development in Coffees Roasted to Different Degrees by Colorimetric Sensor Array,” Food Chem. 240:808-816 (2018), which is hereby incorporated by reference in its entirety) with standard pattern recognition techniques to determine the sample origin/quality in a semi-quantitative/quantitative manner.

Another aspect of the present application relates to a method of functionalizing a polymer. This method includes:
providing a polymer;
providing a nanocage of Formula (I):

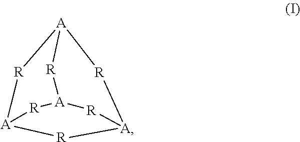

wherein each A is independently selected and has the formula

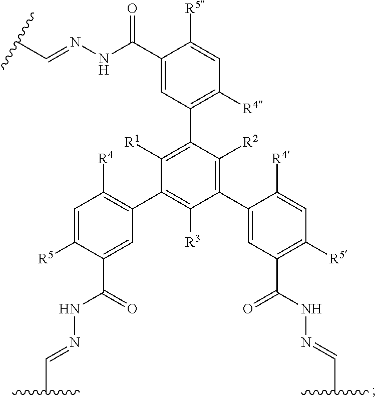

is the point of attachment of A to R;
each R is independently selected and has the formula

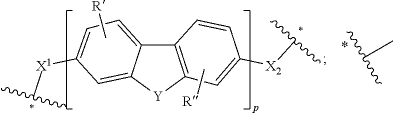

indicates the point of attachment of R to A;
R′ is H or C₁₋₂₀ alkyl;
R″ is H or C₁₋₂₀ alkyl;
R¹, R², and R³ are each independently selected from the group consisting of H, halogen, —OH, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀-alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁴, R^{4′}, and R^{4″} are each independently selected from the group consisting of H, halogen, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OH, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁵, R^{5′}, and R^{5″} are each independently selected from the group consisting of H, halogen, —OH, —O(CH₂)ₙ—(OCH₂CH₂)ₘ—OC₁₋₆ alkyl, —O(CH₂)ₙ—(OCH₂CH₂)ₘ—OH, (OCH₂CH₂)ₘ—OC₁₋₆ alkyl, (OCH₂CH₂)ₘ—OH, aryl, heteroaryl, heterocyclyl, —OC₁₋₂₀ alkyl, —O-perfluorinated C₁₋₂₀alkyl, —Oaryl, and —NR¹⁰R¹¹;
R⁶ and R^{6′} are each independently selected at each occurrence from the group consisting of H, C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl, wherein C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl can be substituted from 1 to 3 times with a substituent selected from the group consisting of —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl;
R¹⁰ and R¹¹ are each independently selected from the group consisting of H, C₁₋₆ alkyl, C₂₋₆ alkenyl, C₂₋₆ alkynyl, aryl, heteroarylalkyl, heterocyclylalkyl, and arylalkyl, wherein each C₁₋₆ alkyl, C₂₋₆ alkenyl, C₂₋₆ alkynyl, aryl, heteroarylalkyl, heterocyclylalkyl, and arylalkyl can be optionally substituted from 1 to 3 times with a substituent selected from the group consisting of H, C₁₋₆ alkyl, heteroarylalkyl, heterocyclylalkyl, and arylalkyl;
X¹ and X² are absent or are independently selected from the group consisting of C₁₋₆ alkylene, C₃₋₈ cycloalkylene, and arylene, wherein C₁₋₆ alkylene, C₃₋₈ cycloalkylene, and arylene can be optionally substituted from 1 to 3 times with H or C₁₋₆ alkyl;
Y is independently selected from the group consisting of —N(R⁶)—, —S—, —O—, —C(R⁶)(R^{6′})—, and —P(R⁶)—;
p is 1 to 5;
n is 1 to 10; and
m is 1 to 50;
providing a functionalizing reagent;
reacting the polymer with the functionalizing reagent within the nanocage having a Formula (I) to produce a functionalized polymer.

According to the present application, in the nanocage of Formula (I), p can be 1, 2, 3, 4, or 5. In one embodiment, p is 1 to 3.

In one embodiment, one kind of polymer is provided. In another embodiment, a mixture of polydisperse polymers is provided.

Polymers that can be functionalized according to the present invention include, but are not limited to, amine-functionalized polyimides, amine-functionalized glycol polymers, amine-functionalized acrylate polymers, amine-functionalized polyolefins, amine-functionalized polyesters, amine-functionalized polylisocyanates, and amine-functionalized polyamides as well as co-polymers thereof.

In another embodiment, the nanocage of Formula (I) has a void space suitable to receive and functionalize the provided polymer.

According to the present application, functionalization of a polymer can be carried out in any suitable solvent, including, but not limited to deuterated dichloromethane (CD₂Cl₂), dichloromethane, deuterated chloroform (CDCl₃), chloroform, pentanes, heptanes, octanes, nonanes, acetonitrile, tetrahydrofuran, ethyl acetate, diethyl ether, dipropyl ether, diphenyl ether, tetrachloroethane, carbon tetrachloride, and nitrobenzene, or the mixture thereof.

Functionalization of a polymer can be carried out at room temperature or at an elevated temperature. Preferably, the temperature is below 60° C., below 55° C., below 50° C., below 45° C., below 40° C., below 35° C., below 30° C. More preferably, functionalization of a polymer is carried out at room temperature.

According to the present application, the nanocage of Formula (I) is provided in the amount of 0.01 wt % to 50 wt %. Preferably, the nanocage of Formula (I) is provided in the amount of 0.01 wt % to 50 wt %, 0.1 wt % to 45 wt %, 1 wt % to 40 wt %, 5 wt % to 40 wt %, 10 wt % to 40 wt %, 15 wt % to 40 wt %, 20 wt % to 40 wt %, 25 wt % to 50 wt %, 30 wt % to 40 wt %. More preferably, the nanocage of Formula (I) is provided in the amount of 0.01 wt % to 30 wt %, 0.01 wt % to 20 wt %, 0.01 wt % to 10 wt %, 0.1 wt % to 10 wt %.

In a further embodiment, the functionalizing reagent is selected from the group consisting of nitrophenyl-3,5-dinitrobenzoate and nitrophenyl acetate.

In yet another embodiment, the polymer is acylated.

The above disclosure is general. A more specific description is provided below in the following examples. The examples are described solely for the purpose of illustration and are not intended to limit the scope of the present application. Changes in form and substitution of equivalents are contemplated as circumstances suggest or render expedient. Although specific terms have been employed herein, such terms are intended in a descriptive sense and not for purposes of limitation.

### EXAMPLES

The following Examples are presented to illustrate various aspects of the present application, but are not intended to limit the scope of the claimed application.

### Example 1—General Methods and Materials

All commercially available starting materials were purchased from Sigma Aldrich, Fisher Scientific, AK Scientific, Alfa Aesar, Ambeed, or Oakwood Chemical. Unless notes otherwise, all reagents were used as received without further purification. When needed, tetrahydrofuran (THF) was dried using a Glass Contour solvent purification system by SG Water USA, LLC. Acetonitrile (CH₃CN), acetic acid (AcOH), dimethyl sulfoxide (DMSO), methanol (MeOH), N,N-dimethylformamide (DMF), dichloromethane (CH₂Cl₂), and diglyme were used as received from Fisher Scientific.

### Reagents and Solvents

The following reagents and solvents were used: mesitylene (Lancaster Synthesis, CAS Registry No. 108-67-8, 98% purity); iodine (Alfa Aesar, CAS Registry No. 7553-56-2, 99.5% purity); periodic acid (Acros Organics, CAS Registry No. 10450-60-9, 99% purity); acetic acid (Fisher Scientific, CAS Registry No. 64-19-7, 99.7% purity); sulfuric acid (Fisher Scientific, CAS Registry No. 7664-93-9, 95% to 98% purity); 2,4-dimethoxybenzeneboronic acid (AK Scientific, CAS Registry No. 133730-34-4, 98% purity); barium hydroxide octahydrate (Alfa Aesar, CAS Registry No. 12230-71-6, 98% purity); diglyme (Fisher Scientific, CAS Registry No. 111-96-6, 99% purity); tetrakis(triphenylphosphine)palladium(0) (Ambeed, CAS Registry No. 14221-01-3, 98% purity); dichloromethane (Fisher Scientific, CAS Registry No. 75-09-2, 99.5% purity); dimethylformamide (Fisher Scientific, CAS Registry No. 68-12-2, 99.8% purity); phosphorus oxychloride (Acros Organics, CAS Registry No. 10025-87-3, 99% purity); acetonitrile (Fisher Scientific, CAS Registry No. 75-05-8, 99.5% purity); dimethyl sulfoxide (Fisher Scientific, CAS Registry No. 67-68-5, 99.9% purity); sodium phosphate monobasic (Acros Organics, CAS Registry No. 7558-80-7, 99% purity); sodium chlorite (Aldrich Chemical Company, CAS Registry No. 7758-19-2, 80% purity); boron trichloride solution in dichloromethane (Sigma Aldrich, CAS Registry No. 10294-34-5, 1 M in CH₂Cl₂); tetrahydrofuran (Fisher Scientific, CAS Registry No. 109-99-9); caesium carbonate (AK Scientific, CAS Registry No. 534-17-8, 99.9% purity); hydrazine hydrate (Alfa Aesar, CAS Registry No. 7803-57-8, 98% purity); magnesium sulfate (Fisher Scientific, CAS Registry No. 7487-88-9, AR); trifluoroacetic acid (Fisher Scientific, CAS Registry No. 76-05-1, 99% purity); terephthalaldehyde (Acros Organics, CAS Registry No. 623-27-8, 98% purity); sodium bicarbonate (Oakwood Chemicals, CAS Registry No. 144-55-8, AR); and sodium sulfate (Fisher Scientific, CAS Registry No. 7757-82-6, AR).

### Equipment

All the glassware was from Chemglass. Magnetic stirring apparatus used were Chemglass arex 3 digital pro heating magnetic stirrer CG-1994-V or Chemglass optimag+safety control. Column chromatography was performed on a Teledyne CombiFlash® Rf+ chromatography system. Melting points were obtained using a Digimelt SRS MPA160 melting point apparatus. Evaporation of solvents was achieved using Buchi R-210 rotary evaporator.

¹H NMR spectra were recorded at 298 K on a Varian Unity Inova 500 (500 MHz) spectrometer, a Bruker ARX 500 (500 MHz) spectrometer or, a Bruker Avance-III-800 (800 MHz) spectrometer. ¹³C (¹H) NMR spectra were recorded at 298 K on a Bruker ARX 500 (125 MHz) spectrometer or a Bruker Avance-III-800 (200 MHz) spectrometer with a QCI cryoprobe. Samples for NMR spectroscopy were dissolved in CDCl₃, CD₂Cl₂, or DMSO-d₆. The spectra were referenced to the residual solvent peak (CDCl₃: 7.26 ppm for ¹H and 77.16 ppm for ¹³C (¹H) NMR), or to tetramethylsilane (TMS, 0.00 ppm for ¹H and ¹³C (¹H) NMR) as the internal standard. Chemical shift values are reported in parts per million (ppm). Data are reported as follows: chemical shift, multiplicity (s=singlet, d=doublet, t=triplet, q=quartet, m=multiplet, br=broad peak), coupling constants (Hz), and number of protons. The raw ¹H NMR and ¹³C (¹H) NMR spectral data were processed and analyzed with the MestReNova software suite (version 14.0.1).

### Reaction Setup/Workup and Chromatography

If necessary, air and/or moisture sensitive reactions were carried out under an inert atmosphere of argon. Removal of solvents was accomplished on a Buchi R-210 rotary evaporator and further concentration was attained under a Fisher Scientific Maxima C-Plus vacuum line. Column chromatography was performed automatically with a Teledyne CombiFlash® Rf+ chromatography system.

### Example 2—Synthesis of 1,3,5-Triiodo-2,4,6-trimethylbenzene (2)

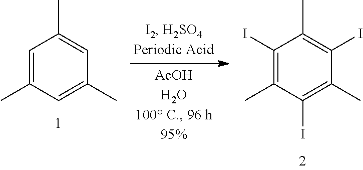

Mesitylene (1) (6.4 g, 7.41 mL, 53.2 mmol), iodine (54.1 g, 213 mmol), and periodic acid (9.7 g, 42.6 mmol) were added to a 500 mL round bottom flask charged with a magnetic stir bar, followed by the addition of acetic acid (100 mL), water (20.0 mL), and sulfuric acid (3.2 mL, 60.2 mmol). A reflux condenser was attached to the flask and the solution was stirred at 100° C. for 72 hours. Afterwards, the reaction mixture was diluted with water (250 mL) and the resulting precipitate was filtered off using a 150 mL sintered filtration funnel. The precipitate was washed with water to remove the acid and then washed with acetone to give 1,3,5-triiodo-2,4,6-trimethylbenzene (2) as a slightly pink solid (FIG. 2). After drying under vacuum overnight the solid became colorless (25.2 g, 95%). The characterization data matched the data reported in the literature (Yang et al., “Methyl-Restricted Rotor Rotation on the Stator Produces High-Efficiency Fluorescence Emission: A New Strategy to Achieve Aggregation-Induced Emission,” RSC Adv. 9:12078-12084 (2019), which is hereby incorporated by reference in its entirety). ¹H NMR (500 MHz, CDCl₃) δ (ppm)=3.01 (s, 9H). ¹³C (¹H) NMR (125 MHz, CDCl₃) δ (ppm)=144.15, 101.16, 39.56.

### Example 3—Synthesis of Syn-5′-(2,4-dimethoxyphenyl)-2,2″,4,4′-tetramethoxy-2′,4′,6′-trimethyl-1,1′:3′,1″-terphenyl (Syn-3)

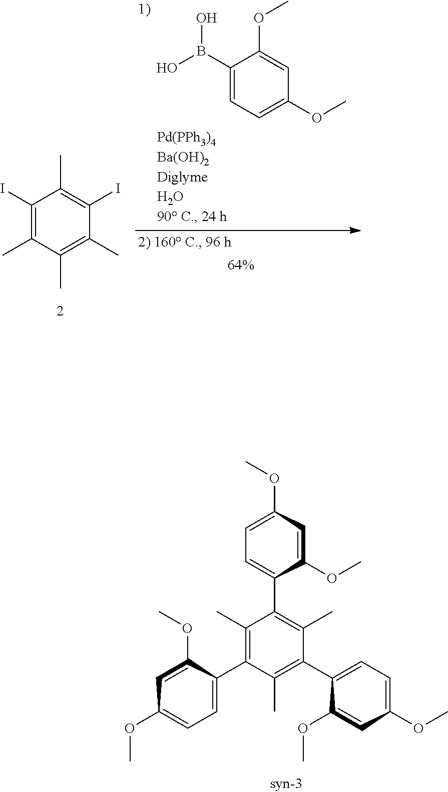

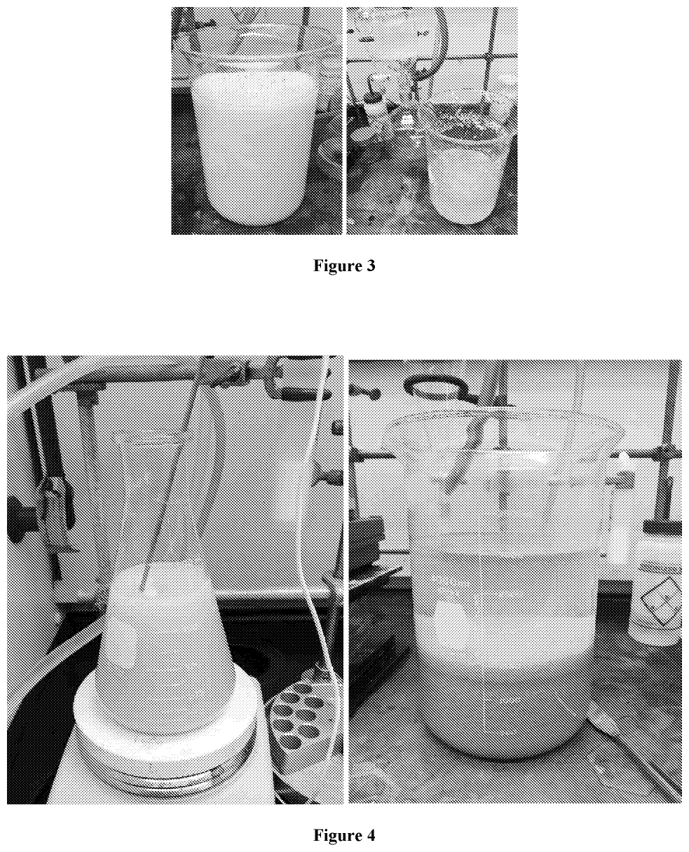

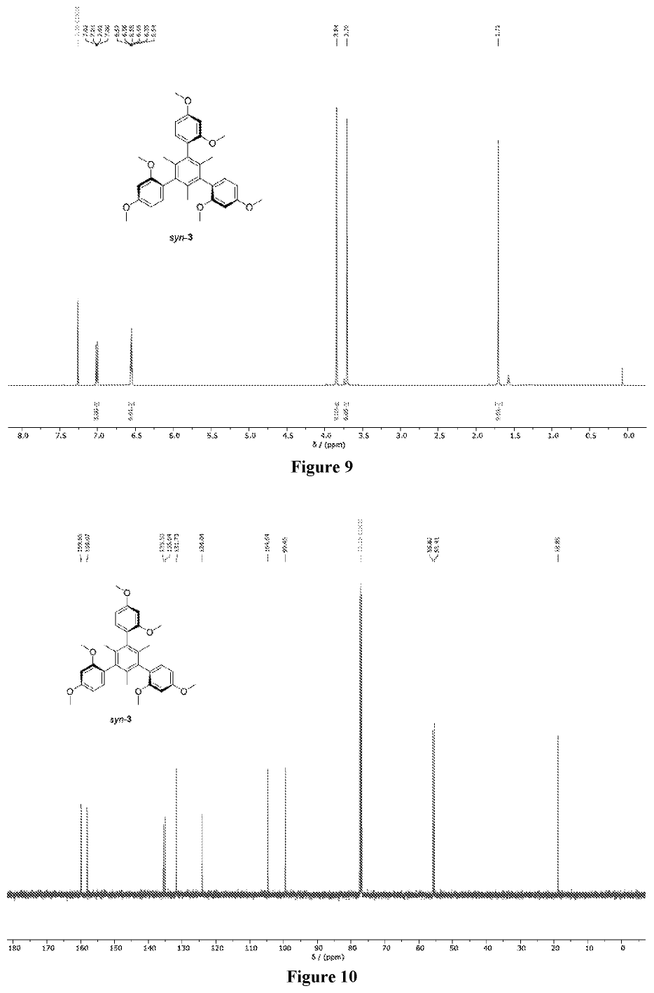

1,3,5-Triiodo-2,4,6-trimethylbenzene (2, 20.0 g, 40.2 mmol), 2,4-dimethoxyphenylboronic acid (35.5 g, 195 mmol), and barium hydroxide octahydrate (110 g, 348 mmol) were suspended in a mixture of diglyme (500 mL) and water (100 mL) in a 1000 mL round bottom flask charged with a magnetic stir bar. After purging the suspension with argon for 3 minutes, tetrakis(triphenylphosphine) palladium(0) (2.32 g, 2.01 mmol) was added to the reaction flask followed by further purging with Argon for 5 additional minutes, at which point the flask was capped with a silicon stopper and fitted a balloon filled with argon. The reaction mixture was then heated to 90° C. and stirred for 24 hours. After 24 hours, the reaction mixture was allowed to cool to room temperature and filtered through celite. The filtrate was then poured into deionized water (3 L) and left to precipitate overnight (FIG. 3). The precipitate was a yellow slimy goo which was filtered off using a 350 mL sintered funnel and transferred to a 500 mL Erlenmeyer flask. The funnel was rinsed with a small amount of CH₂Cl₂ to wash out the last of the compound. The CH₂Cl₂ was evaporated by adding the flask to a hotplate and setting it to 50° C. When the mixture stopped boiling, ethanol (300 mL) was added to the flask, the hotplate was set to 80° C. and the mixture was brought to boil (FIG. 4). After boiling for approximately 2 minutes the flask was taken off the hotplate and allowed to cool to room temperature over 24 hours. The precipitate was filtered off leaving a colorless solid. The solid was added to three 20 mL vials and heated to 160° C. for 96 hours, converting all of the compound to the syn-confirmation. As the compound heated up it would melt into a liquid and after about 24 hours of heating it became a solid again. After heating, the compound was again transferred to an Erlenmeyer flask which was added to a hotplate. Ethanol (150 mL) was then added to the flask, the hotplate was set to 80° C., and the mixture was brought to a boil. After boiling for approximately 2 minutes the flask was taken off the hotplate and allowed to cool to room temperature over 24 hours. The precipitate was filtered off providing syn-3 (13.6 g, 64%) as a colorless solid. The characterization data matched the data reported in the literature (Sharafi et al., “Crystal-Packing-Driven Enrichment of Atropoisomers,” Angew. Chem., Int. Ed. 56:7097 7101 (2017), which is hereby incorporated by reference in its entirety). ¹H NMR (500 MHz, CDCl₃) δ=7.04-6.99 (m, 3H), 6.58-6.52 (m, 6H), 3.84 (s, 9H), 3.70 (s, 9H), 1.71 (s, 9H) (FIG. 9). ¹³C (¹H) NMR (125 MHz, CDCl₃)=δ 159.73, 157.95, 135.38, 134.92, 131.61, 123.93, 104.52, 99.33, 55.70, 55.29, 18.74 (FIG. 10). HRMS (ESI+) m/z: calcd for C₃₃H₃₆O₆ [M+H]⁺ 529.2590, found 529.2604.

### Example 4—Synthesis of Syn-5′-(5-formyl-2,4-dimethoxyphenyl)-4,4″,6,6′-tetramethoxy-2′,4′,6′-trimethyl-[1,1′:3′,1″-terphenyl]-3,3″-dicarbaldehyde (Syn-4)

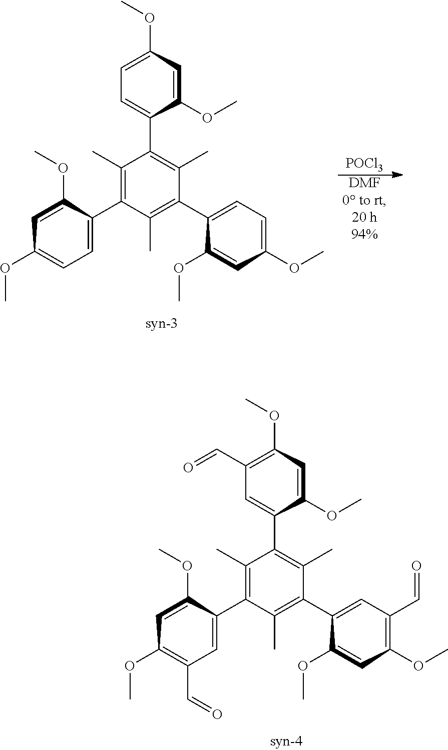

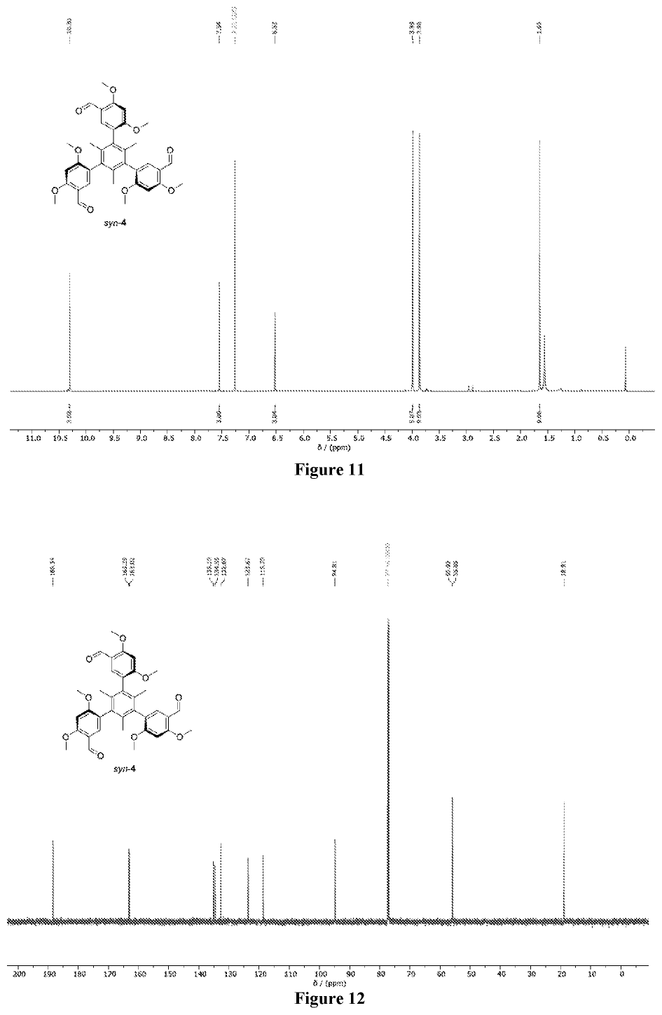

Syn-3 (13.3 g, 25.2 mmol) was dissolved in anhydrous DMF (500 mL) followed by the addition of phosphorus oxychloride (60 mL, 644 mmol) at room temperature. After 2 hours another batch of phosphorus oxychloride (20 mL, 215 mmol) was added and the reaction was left to stir at room temperature for 20 hours. After 20 hours the reaction was analyzed by Thin Layer Chromatography (TLC) and it found that it was not yet complete. Therefore, another batch of phosphorus oxychloride (20 mL, 215 mmol) was added. The reaction was left to stir for 48 hours at room temperature and again TLC showed the reaction was not complete and another batch of phosphorus oxychloride (20 mL, 215 mmol) was added and the reaction mixture was left to stir for another 2 hours. Next, the reaction was again analyzed by TLC, which now showed full conversion and the reaction mixture was quenched by pouring it into 3 L of ice water. The mixture was left overnight to allow for the product to completely precipitate out (FIG. 3, right panel). The precipitated solid was filtered off using a 350 mL sintered funnel, washed with deionized water, and left to dry under high vacuum overnight to afford syn-4 as a colorless solid (14.6 g, 94%). The characterization data matched the data reported in the literature (Sharafi et al., “Size-Selective Catalytic Polymer Acylation with a Molecular Tetrahedron,” Chem 6:1469-1494 (2020), which is hereby incorporated by reference in its entirety). ¹H NMR (500 MHz, CDCl₃) d=10.30 (s, 3H), 7.54 (s, 3H), 6.52 (s, 3H), 3.99 (s, 9H), 3.86 (s, 9H), 1.65 (s, 9H) (FIG. 11). ¹³C NMR (125 MHz, CDCl₃) d=188.3, 163.2, 162.9, 135.1, 134.5, 132.7, 123.6, 118.7, 94.8, 55.9, 55.8, 18.8 (FIG. 12). FIRMS (ESI+) m/z: calcd for C₃₆H₃₆O₉ [M+H]⁺ 613.2438, found 613.2452.

### Example 5—Synthesis of Syn-5′-(5-carboxy-2,4-dimethoxyphenyl)-4,4″,6,6″-tetramethoxy-2′,4′,6′-trimethyl-[1,1′:3′,1″-terphenyl]-3,3″-dicarboxylic acid (Syn-5)

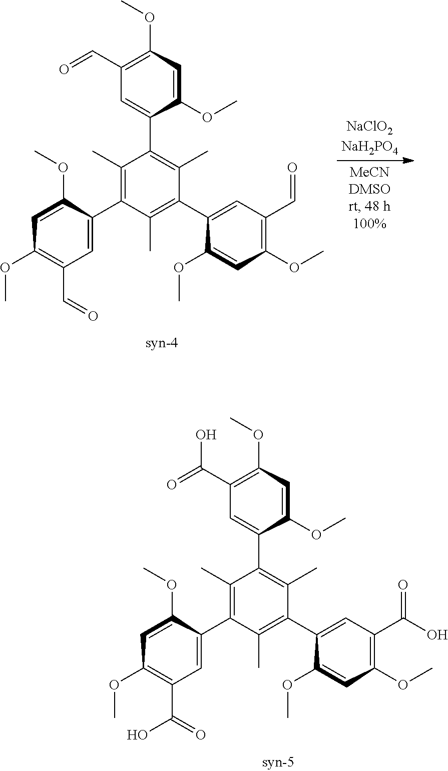

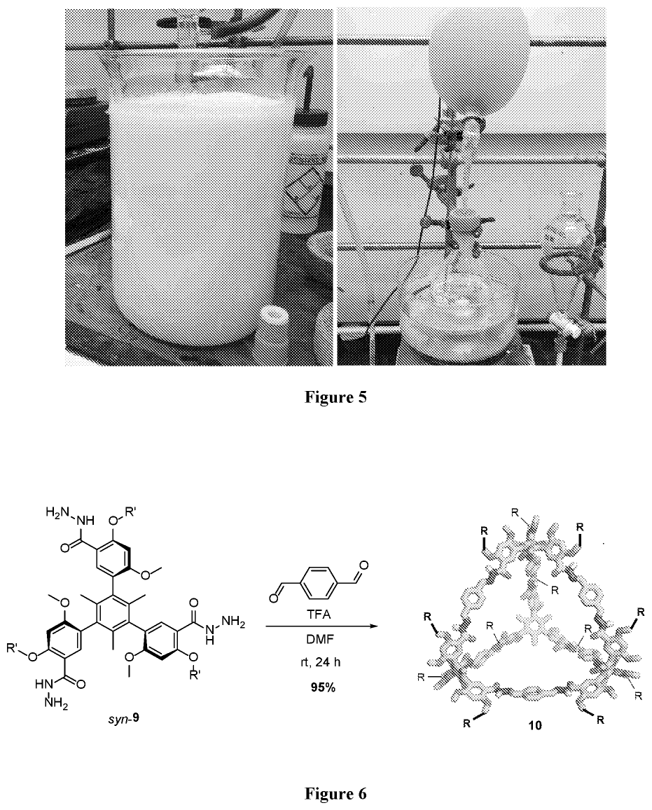

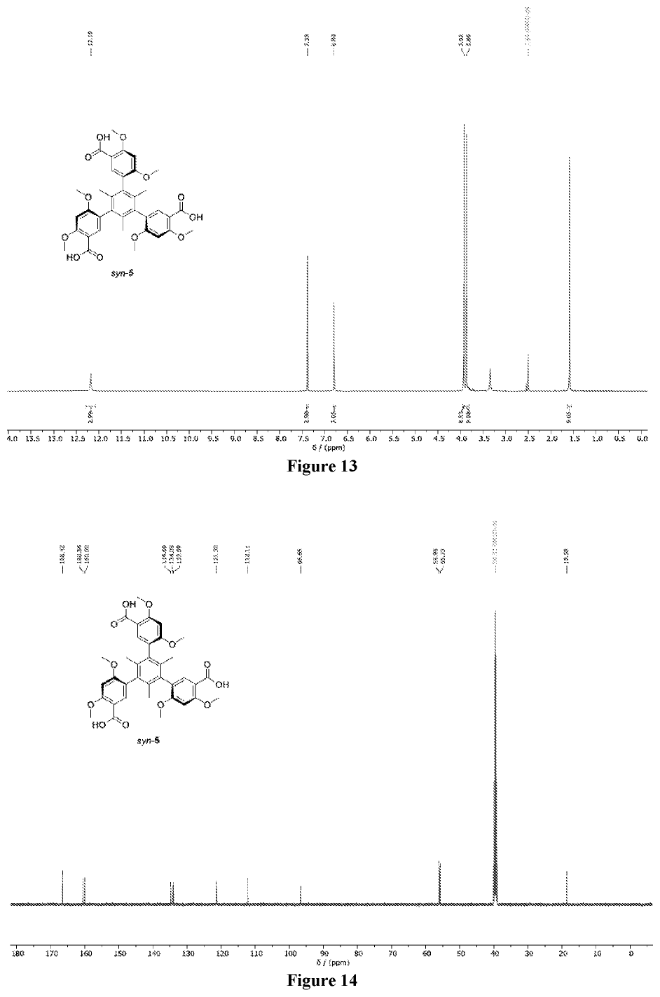

Syn-4 (14.0 g, 22.9 mmol) was dissolved in a 500 mL of a 1:1 vol % MeCN/DMSO mixture in a 1000 mL round-bottomed flask. Next, sodium phosphate monobasic (32.9 g, 274 mmol) and sodium chlorite (tech., nominally, 80%, 24.8 g, 274 mmol) were dissolved in deionized water (150 mL) and added to the reaction mixture, which led the solution to go from colorless to an orange color. The reaction temperature increased significantly, and the flask was cooled in an ice bath for 1 hour. Afterwards, the ice bath was removed, and the reaction was allowed to stir at room temperature for 48 hours at which point the solution was poured into 3 L of 1% HCl in water and was allowed to precipitate out for 24 hours (FIG. 5, left panel). The precipitate was filtered off using a 350 mL sintered funnel and washed with deionized water. The precipitated solid was added to a 500 mL round bottomed flask and dried under vacuum overnight providing syn-5 a colorless solid (15.1 g, 100%). ¹H NMR (500 MHz, DMSO-d6) δ=12.17 (s, 3H), 7.37 (s, 3H), 6.79 (s, 3H), 3.91 (s, 9H), 3.85 (s, 9H), 1.57 (s, 9H) (FIG. 13). ¹³C (¹H) NMR (125 MHz, DMSO-d6) δ=166.88, 161.00, 160.46, 135.15, 134.55, 134.35, 121.76, 112.57, 97.11, 56.41, 56.19, 19.04 (FIG. 14). FIRMS (ESI+) m/z: calcd for C₃₆H₃₆O₁₂ [M+Na]⁺ 683.2104, found 683.2106.

### Example 6—Synthesis of Syn-dimethyl 5′-(2,4-dimethoxy-5-(methoxycarbonyl)phenyl)-4,4″,6,6′-tetramethoxy-2′,4′,6′-trimethyl-[1,1′:3′,1″-terphenyl]-3,3″-dicarboxylate (Syn-6)

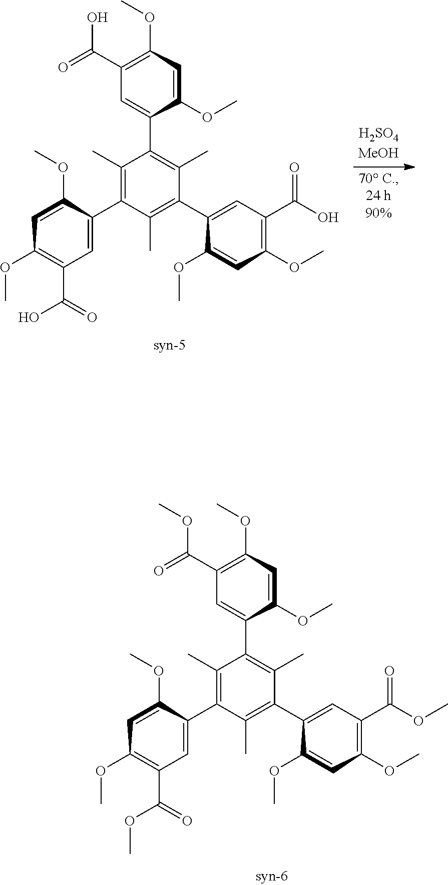

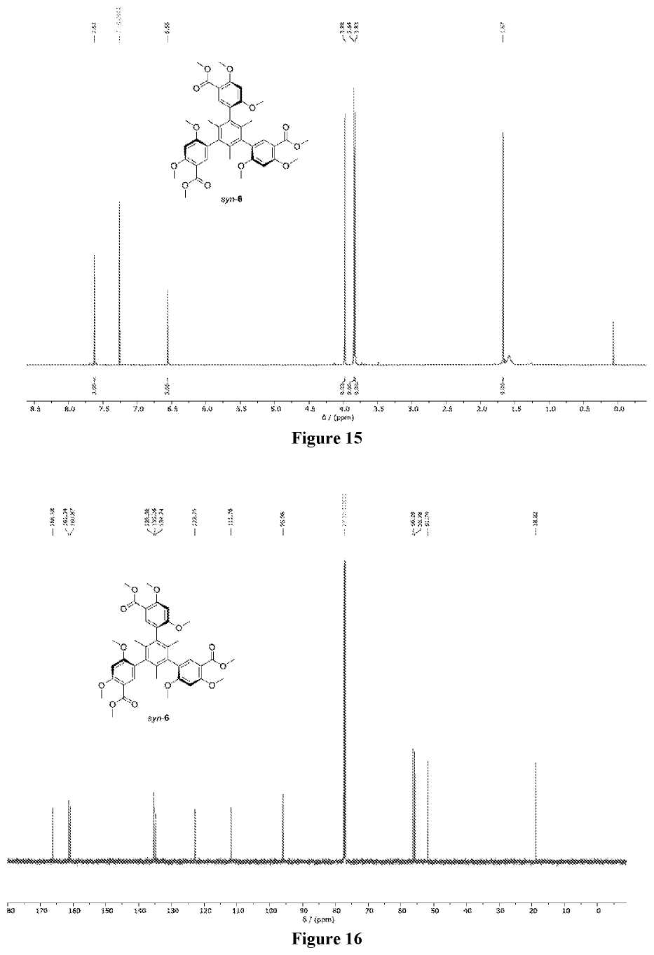

Syn-5 (15.0 g, 22.7 mmol) was dissolved in MeOH (750 mL) in a 1000 mL round-bottomed flask, followed by the dropwise addition of concentrated sulfuric acid (28.4 mL, 534 mmol). A reflux condenser was added, and the reaction mixture was stirred at reflux (at around 70° C.) for 24 hours. The solution was then poured into 3 L of deionized water and allowed to precipitate overnight. The white precipitate was filtered off and washed with deionized water and then dried under vacuum overnight to provide syn-6 as a colorless solid (14.4 g, 90%). ¹H NMR (500 MHz, CDCl₃) δ=7.62 (s, 3H), 6.56 (s, 3H), 3.98 (s, 9H), 3.84 (s, 9H), 3.83 (s, 9H), 1.67 (s, 9H) (FIG. 15). ¹³C (¹H) NMR (125 MHz, CDCl₃) δ=166.07, 161.23, 160.76, 135.25, 135.15, 134.63, 122.64, 111.67, 95.85, 56.18, 55.67, 51.63, 18.71 (FIG. 16). HRMS (ESI+) m/z: calcd for C₃₉H₄₂O₁₂ [M+H]⁺ 703.2755, found 703.2755.

### Example 7—Synthesis of Syn-dimethyl 4,4″-dihydroxy-5′-(4-hydroxy-2-methoxy-5-(methoxycarbonyl)phenyl)-6,6′-dimethoxy-2′,4′,6′-trimethyl-[1,1′:3′,1″-terphenyl]-3,3″-dicarboxylate (Syn-7)

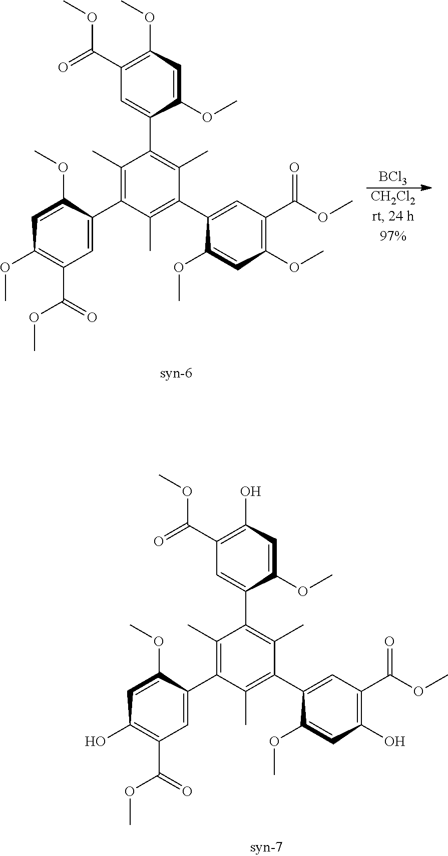

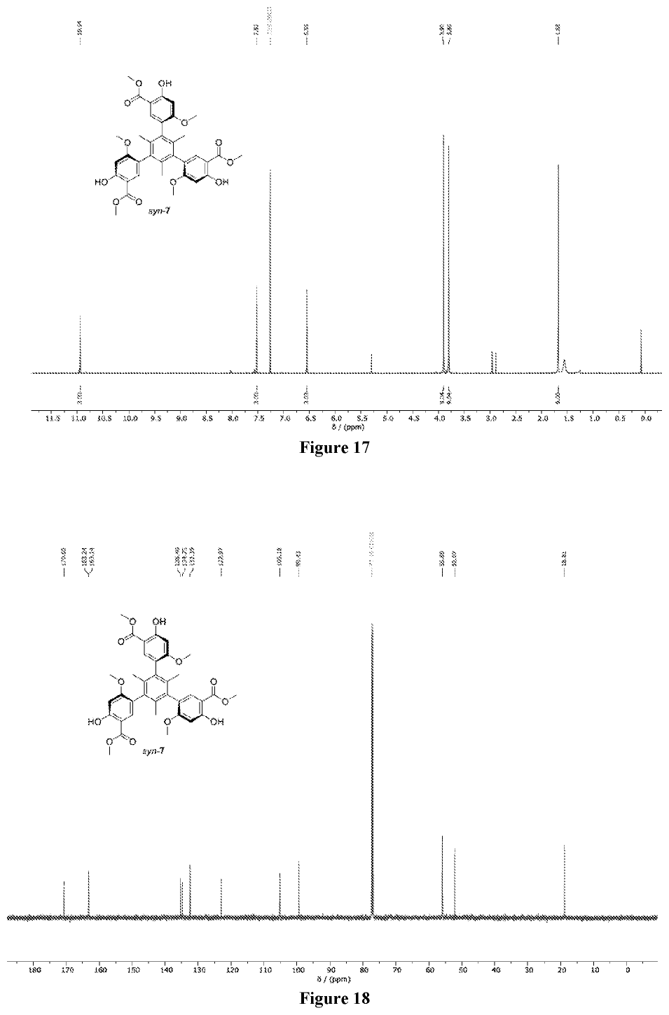

Syn-6 (14.2 g, 20.2 mmol) was dissolved in CH₂Cl₂ (500 mL) in a 1000 mL round bottom flask. Next, a 1 M Boron trichloride solution in CH₂Cl₂ (90 mL, 90 mmol) was added by syringe to the reaction mixture under an argon atmosphere. After stirring at room temperature for 24 hours, deionized water (50 mL) was added to quench the reaction. The flask with the resulting biphasic mixture was attached to a rotary evaporator and the CH₂Cl₂ was evaporated leading the product to precipitate out. The product was filtered off using a 350 mL sintered funnel, providing a grey solid. The precipitate was added to a 500 mL round bottom flask and dried under vacuum overnight to afford syn-7 (13.3 g, 97%). ¹H NMR (500 MHz, CDCl₃) δ=10.94 (s, 3H), 7.52 (s, 3H), 6.55 (s, 3H), 3.90 (s, 9H), 3.80 (s, 9H), 1.68 (s, 9H) (FIG. 17). ¹³C (¹H) NMR (125 MHz, CDCl₃) δ=170.52, 163.11, 163.01, 135.27, 134.62, 132.26, 122.84, 105.05, 99.29, 55.75, 51.94, 18.68 (FIG. 18). FIRMS (ESI+) m/z: calcd for C₃₆H₃₆O₁₂ [M+H]⁺ 661.2285, found 661.2286.

### Example 8—Synthesis of Syn-dimethyl 4,4″-bis((2,5,8,11-tetraoxapentadecan-15-yl)oxy)-5′-(4-((2,5,8,11-tetraoxapentadecan-15-yl)oxy)-2-methoxy-5-(methoxycarbonyl)phenyl)-6,6′-dimethoxy-2′,4′,6′-trimethyl-[1,1′:3′,1″-terphenyl]-3,3″-dicarboxylate (Syn-8)

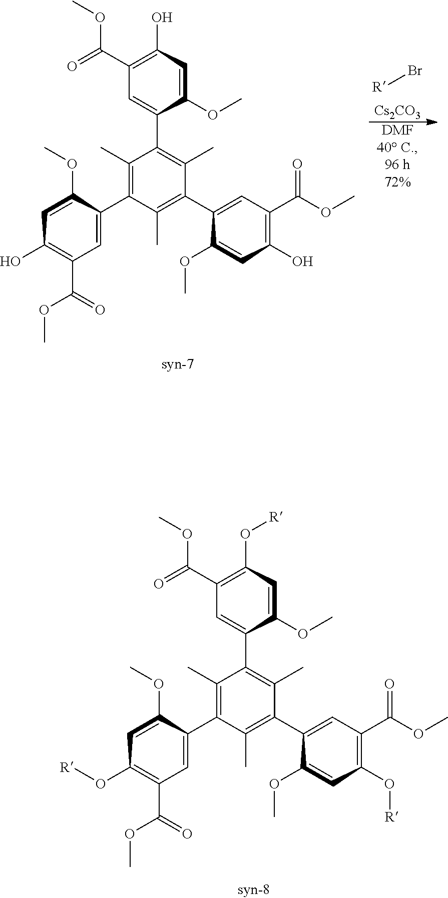

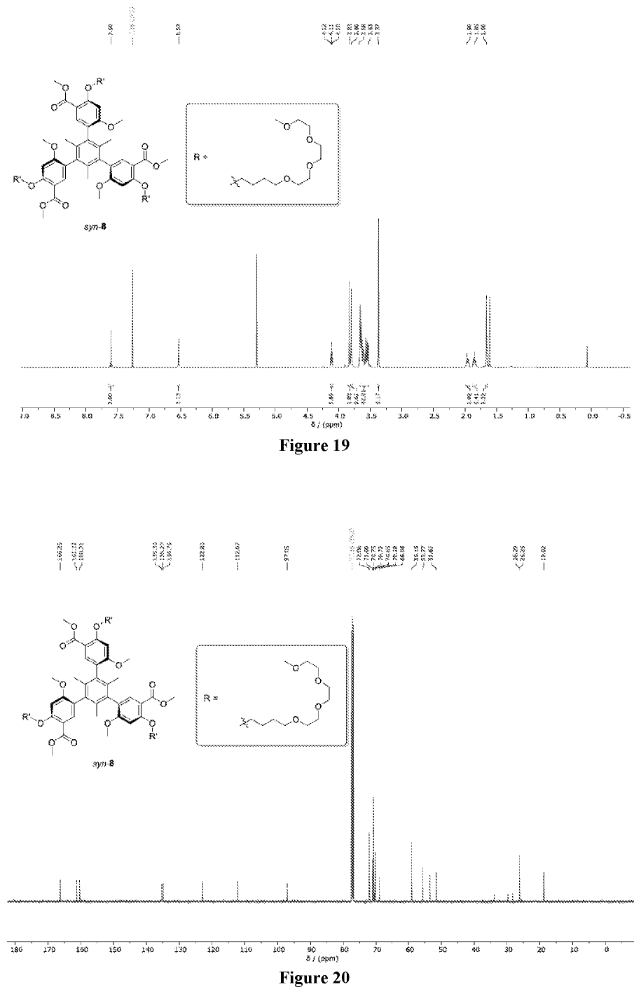

Syn-7 (3.00 g, 4.54 mmol) and cesium carbonate (7.40 g, 22.7 mmol) were added to a 500 mL round-bottomed flask along with DMF (180 mL) followed by the addition of 15-bromo-2,5,8,11-tetraoxapentadecane (4.35 g, 14.5 mmol). The reaction mixture was heated to 40° C. and stirred for 72 hours. Then, the reaction mixture was transferred to a 1000 mL separatory funnel and 200 mL of deionized water was added. Finally, the mixture was extracted with EtOAc (3×100 mL), the organic phases were combined in the separatory funnel and the reaction was washed four times with 150 mL of 20% brine solution to remove any remaining DMF. The organic phase was then dried over anhydrous Na₂SO₄, filtered, and evaporated under reduced pressure. The crude product was purified by flash column chromatography over silica gel using a Teledyne CombiFlash® Rf+ chromatography system (eluent: 10 vol % MeOH in CH₂Cl₂) to afford syn-8 as a pale-yellow oil (4.32 g, 72%). The characterization data matched the data reported in the literature (Sharafi et al., “Size-Selective Catalytic Polymer Acylation with a Molecular Tetrahedron,” Chem 6:1469-1494 (2020), which is hereby incorporated by reference in its entirety). ¹H NMR (500 MHz, CDCl₃) δ=7.60 (s, 3H), 6.53 (s, 3H), 4.11 (t, J 6.3 Hz, 6H), 3.83 (s, 9H), 3.80 (s, 9H), 3.67-3.53 (m, 42H), 1.96 (m, 6H), 1.85 (m, 6H), 3.37 (s, 9H), 1.66 (s, 9H) (FIG. 19). ¹³C (¹H) NMR (125 MHz, CDCl₃) δ=166.13, 161.11, 160.19, 135.19, 135.08, 134.67, 122.69, 111.96, 96.94, 71.94, 70.88, 70.64, 70.61, 70.54, 70.07, 68.87, 59.03, 55.66, 51.51, 26.18, 26.13, 18.71 (FIG. 20). FIRMS (ESI+) m/z: [M+NH₄]⁺ calcd 1332.7128, found 1332.7105.

### Example 9—Synthesis of Syn-4,4″-bis((2,5,8,11-tetraoxapentadecan-15-yl)oxy)-5′-(4-((2,5,8,11-tetraoxapentadecan-15-yl)oxy)-5-(hydrazinecarbonyl)-2-methoxyphenyl)-6,6′-dimethoxy-2′,4′,6′-trimethyl-[1,1′:3′,1″-terphenyl]-3,3″-dicarbohydrazide (Syn-9)

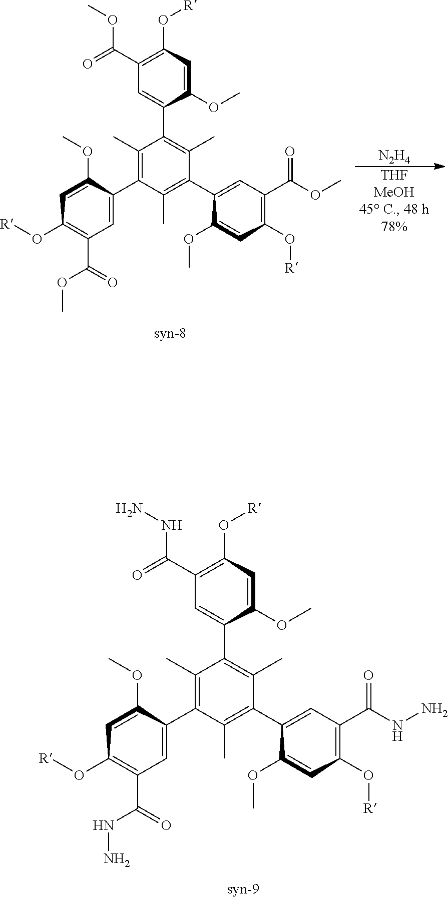

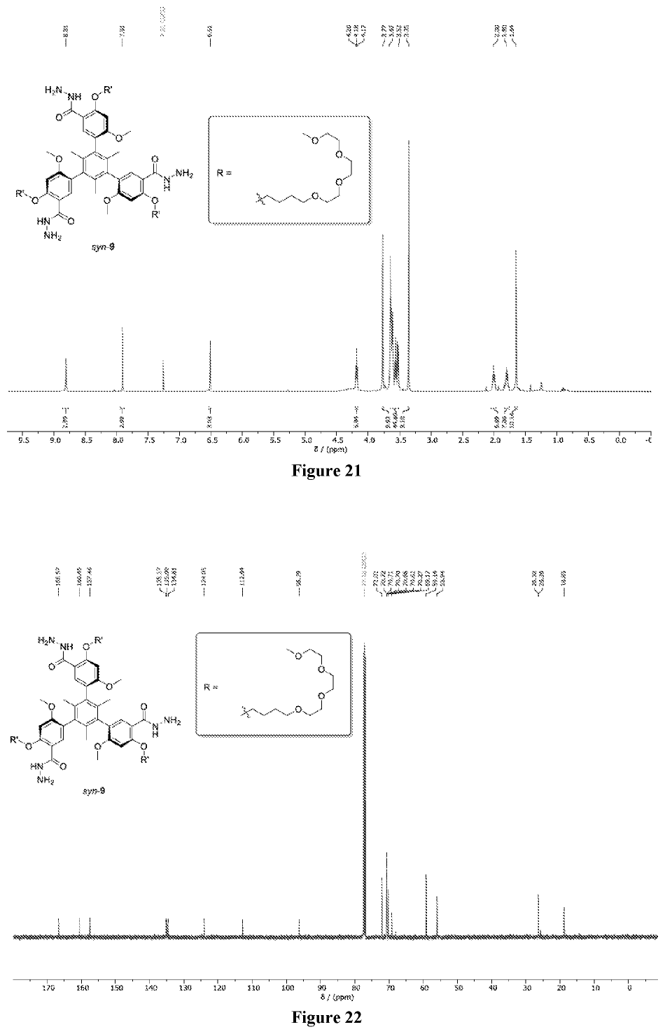

Syn-8 (4.00 g, 3.04 mmol) was dissolved in THF (85 mL) and MeOH (170 mL) in a 500 mL round bottom flask. Next, the reaction mixture was degassed with argon and hydrazine hydrate (47.7 mL, 1.52 mol) was added. The reaction was heated to 45° C. and allowed to stir for 48 hours under an argon atmosphere. After 48 hours, the solvent was evaporated under reduced pressure and the resulting mixture was transferred to a 500 mL separatory funnel and extracted with CH₂Cl₂ (3×50 mL). The combined organic layers were washed with water (3×75 mL) to remove excess hydrazine, dried over anhydrous Na₂SO₄, filtered using a 150 mL sintered funnel, concentrated under reduced pressure, and dried in vacuo overnight to afford syn-9 as a yellow oil (3.11 g, 78%). The characterization data matched the data reported in the literature (Sharafi et al., “Size-Selective Catalytic Polymer Acylation with a Molecular Tetrahedron,” Chem 6:1469 1494 (2020), which is hereby incorporated by reference in its entirety). ¹H NMR (500 MHz, CDCl₃) δ=8.81 (s, 3H), 7.91 (s, 3H), 6.51 (s, 3H), 4.18 (t, J=6.6 Hz, 6H), 3.77 (s, 9H), 3.67-3.52 (m, 42H), 3.35 (s, 9H), 2.00 (m, 6H), 1.80 (m, 6H), 1.64 (s, 9H) (FIG. 21). ¹³C (¹H) NMR (125 MHz, CDCl₃) δ=166.47, 160.35, 157.36, 135.07, 134.90, 134.51, 123.94, 112.73, 96.19, 71.92, 70.62, 70.60, 70.59, 70.55, 70.52, 70.16, 69.07, 59.03, 55.83, 26.21, 26.18, 18.75 (FIG. 22). HRMS (ESI+) m/z: [M+H]⁺ calcd 1315.7176, found 1315.7200.

### Example 10—Synthesis of Hydrazone-Linked Tetrahedron 10

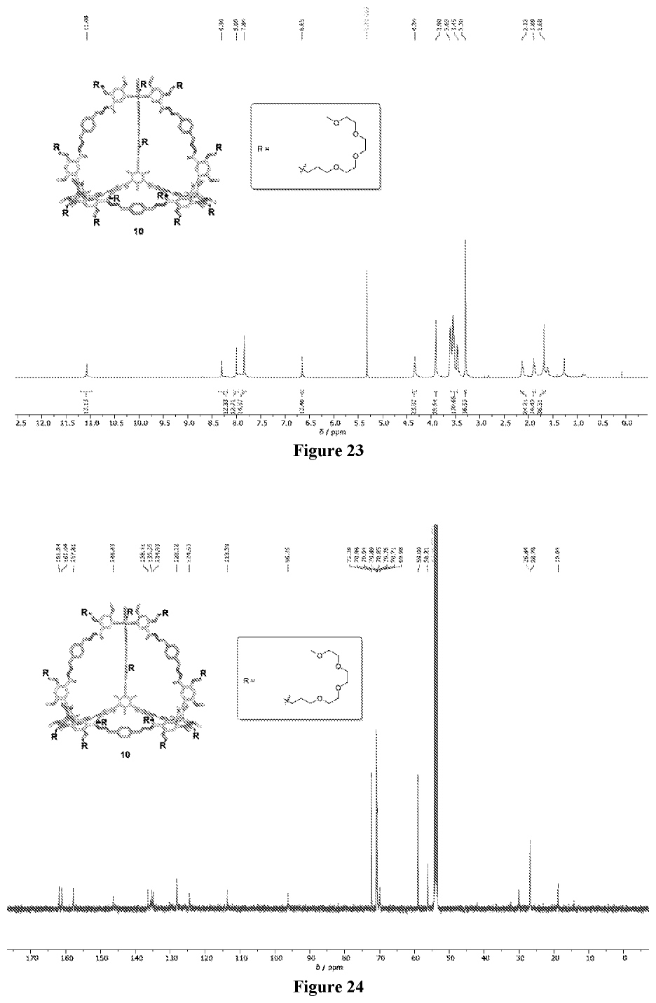

Syn-9 (2.41 g, 1.83 mmol) was dissolved in DMF (400 mL), and the solution was degassed with argon. Next, terephthalaldehyde (369 mg, 2.75 mmol) and trifluoro acetic acid (140 uL, 1.83 mmol) were added consecutively, and the reaction mixture was stirred at room temperature under an argon atmosphere for 48 hours. After 48 hours, the mixture was poured into 2 L of a 1% aqueous solution of sodium bicarbonate and allowed to precipitate for 24 hours at which point the precipitate was filtered off using a 350 mL sintered funnel. The yellow precipitate was dissolved in CH₂Cl₂, filtered again, evaporated to dryness under reduced pressure, and dried under high vacuum to provide the hydrazone-linked tetrahedron 10 as a yellow solid (2.55 g, 95%) (FIG. 6). The characterization data matched the data reported in the literature (Sharafi et al., “Size-Selective Catalytic Polymer Acylation with a Molecular Tetrahedron,” Chem 6:1469-1494 (2020), which is hereby incorporated by reference in its entirety). ¹H NMR (500 MHz, CD₂Cl₂) δ=11.08 (s, 12H), 8.30 (s, 12H), 8.00 (s, 12H), 7.84 (s, 24H), 6.65 (s, 12H), 4.34 (t, J=6.6 Hz, 24H), 3.90 (s, 36H), 3.63-3.45 (m, 168H), 3.30 (s, 36H), 2.13 (m, 24H), 1.89 (m, 24H), 1.68 (s, 36H) (FIG. 23). ¹³C (¹H) NMR (125 MHz, CD₂Cl₂) δ 161.42, 160.63, 157.40, 146.02, 136.00, 135.23, 134.94, 134.51, 127.70, 124.11, 113.18, 95.84, 71.87, 70.55, 70.53, 70.48, 70.43, 70.35, 70.30, 69.57, 58.58, 55.80, 26.43, 26.36, 18.43 (FIG. 24). HRMS (ESI+) m/z: [M+5H]⁵⁺ calcd 1,170.9964, found 1,170.9982.

### Example 11—Method Description

The synthesis started with iodination of the commercially available mesitylene, 1, to form 1,3,5-triiodomesitylene (2), achieved via an electrophilic aromatic substitution reaction using periodic acid and iodine (25.2 g, 95% yield) using a modified literature procedure (Yang et al., “Methyl-Restricted Rotor Rotation on the Stator Produces High-Efficiency Fluorescence Emission: A New Strategy to Achieve Aggregation-Induced Emission,” RSC Adv. 9:12078-12084 (2019), which is hereby incorporated by reference in its entirety). A Suzuki reaction between 2 and the commercially available 2,4-dimethoxyphenylboronic acid then produced a mixture of the syn and anti-isomers of compound 3, which was simply heated to achieve a solid-state-driven amplification of the syn-atropoisomer (13.6 g, 64% yield). While this unique solid-state driven amplification of a minor atropostereoisomer was reported previously (Sharafi et al., “Crystal-Packing-Driven Enrichment of Atropoisomers,” Angew. Chem., Int. Ed. 56:7097-7101 (2017), which is hereby incorporated by reference in its entirety), the procedure has been i) scaled up to prepare over 10 g of material in a single batch and ii) simplified by eliminating the need for chromatographic purification of the crude syn/anti-mixture of compound 3. Syn-3 was then formylated using phosphorus oxychloride to reach syn-4 (14.6 g, 94% yield) (Sharafi et al., “Size-Selective Catalytic Polymer Acylation with a Molecular Tetrahedron,” Chem 6:1469-1494 (2020), which is hereby incorporated by reference in its entirety). Next, a Pinnick oxidation was used to convert the aldehydes to carboxylic acids, producing syn-5 (15.1 g, 100% yield). The carboxylic acids subsequently underwent an esterification to methyl esters, to yield compound syn-6 (14.4 g, 90% yield). Finally, the methoxyl groups ortho to the aldehydes of the methyl ester functions were removed selectively with BCl₃ to afford the tris-phenol syn-7 (13.3 g, 97%), which can serve as a common late-stage intermediate for the synthesis of a large family of future nanocages with different peripheral functional groups. Ultimately, an optimized large-scale procedure to convert syn-7 into our original, hydrazone-linked tetrahedron 10 was also developed (Sharafi et al., “Size-Selective Catalytic Polymer Acylation with a Molecular Tetrahedron,” Chem 6:1469-1494 (2020), which is hereby incorporated by reference in its entirety). Notably, this new procedure to form the nanocage 10 uses DMF instead of CH₂Cl₂ as the solvent, which allowed to simply precipitate the nanocage 10 with water from the crude reaction mixture. Furthermore, the solvent volume required for nanocage formation was also reduced by a factor of 6 compared to the original reported procedure (Sharafi et al., “Size-Selective Catalytic Polymer Acylation with a Molecular Tetrahedron,” Chem 6:1469-1494 (2020), which is hereby incorporated by reference in its entirety), resulting in a much more cost-effective synthesis of the nanocage 10 (2.1 g, 95% yield).

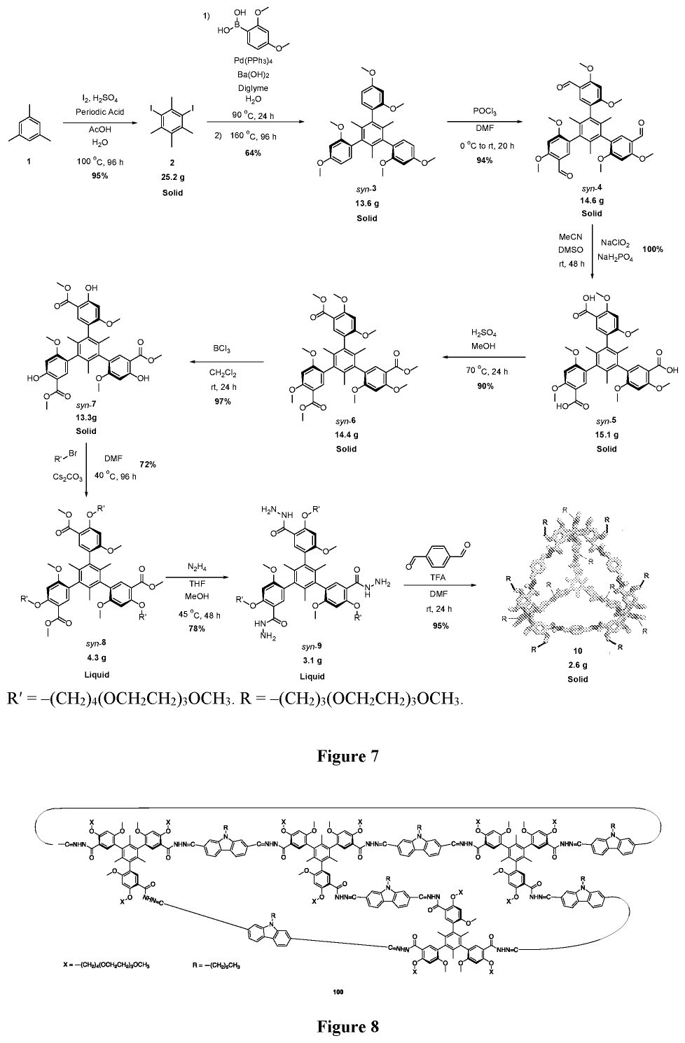

Newly developed synthetic procedure for the hydrazone-linked molecular tetrahedron 10 shown in FIG. 7. Since compounds syn-5, syn-6 and syn-7 were solids, they were purified by simple precipitation/recrystallization techniques to allow for simple and cost-effective scale up.

### Example 12—Results and Discussion of Examples 1-11

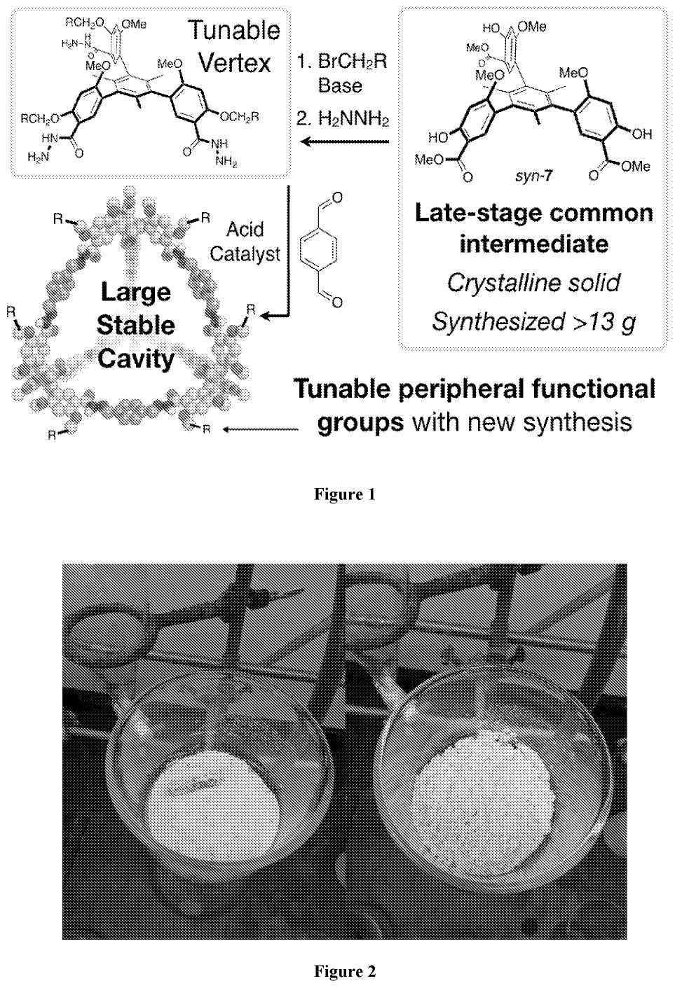

In Sharafi et al., “Size-Selective Catalytic Polymer Acylation with a Molecular Tetrahedron,” Chem 6:1469-1494 (2020), which is hereby incorporated by reference in its entirety, the peripheral triglyme functional groups —R (see also Scheme 1) were attached to the vertices of the tetrahedron at the aldehyde stage, rendering every compound afterwards in the original synthesis an oil with relatively high molecular weight. The physical properties of these original intermediates thus prohibited purification with scalable techniques such as recrystallization or distillation, requiring purification with expensive chromatographic techniques at nearly every step. The new synthesis described in the present application allows for selective deprotection (Scheme 1) of all three ortho-methoxyl groups of the triester vertex syn-6 with BCl₃ in CH₂Cl₂. This provides for an efficient late-stage diversification strategy, since the peripheral triglyme functional groups are now introduced at the latest possible stage in the synthesis. Therefore, this new synthetic strategy provides the vertex syn-7 (FIG. 1) as a common late-stage intermediate for a large family of nanocages with different peripheral functionalization patterns, making it much easier to form cages with different —R groups.

However, the biggest advantage of this new late-stage functionalization approach is that all synthetic intermediates up to compound syn-7 are now crystalline solids, which can be readily purified with simple precipitation/recrystallization techniques. Specifically, it was discovered that compounds syn-5, syn-6, and syn-7 could be purified by simply precipitating them in water and then filtering them off. No further purification was required. The only compound in this new synthesis that still required purification by column chromatography was the triglyme-functionalized vertex syn-8.

The present application describes a facile new synthesis of a covalent, hydrazone-linked molecular tetrahedron, which allowed the production of the catalytically-active (Sharafi et al., “Size-Selective Catalytic Polymer Acylation with a Molecular Tetrahedron,” Chem 6:1469-1494 (2020), which is hereby incorporated by reference in its entirety) tetrahedron for the first time on a multigram scale in a single batch. This new procedure requires a minimal amount of purification as it relies heavily on precipitation in water rendering the synthesis much more practical for large scale. In the nine synthetic steps performed, only one compound required purification by column chromatography. Furthermore, this new synthesis provides access to a common late-stage intermediate, which will allow to readily expand the family of tetrahedral nanocages in the future to tune the peripheral functional groups for selective recognition and catalysis.

### Example 13—Synthesis of 9-Hexyl-9H-carbazole-2,7-dicarbaldehyde (B)

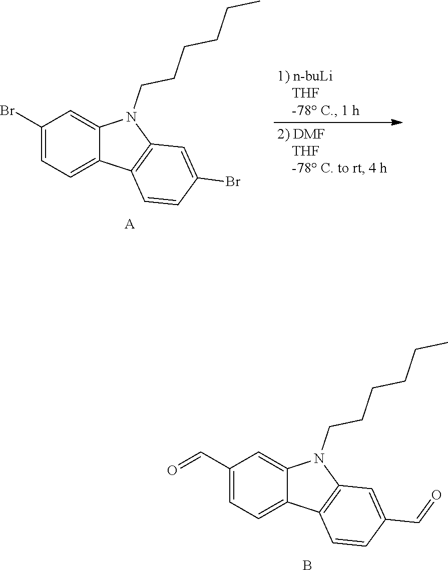

N-Butyllithium (0.269 mL, 2.7 M in toluene, 727 μmol) was added to a solution of 2,7-dibromo-9-hexyl-9H-carbazole (A) (119 mg, 291 μmol) in anhydrous THE (10 mL) at −78° C. under argon atmosphere. The mixture was stirred at −78° C. for 1 hour and then anhydrous dimethylformamide (225 μL, 2.91 mmol) was slowly added. The resulting mixture was stirred for 1 hour at −78° C. and for 3 hours at room temperature. After quenching with water, the mixture was poured into a separatory funnel and extracted with dichloromethane. Next, the combined organic phases were dried using Na₂SO₄ and evaporated in vacuo. The crude product was purified by column chromatography on silica gel using ethyl acetate/hexane (1:10) as the eluent to afford 40 mg of 9-hexyl-9H-carbazole-2,7-dicarbaldehyde (B) as a yellow solid in 45% yield. ¹H NMR (500 MHz, CDCl₃) δ=10.20 (s, 2H), 8.29 (d, J=9.0 Hz, 2H), 8.01 (s, 2H), 7.80 (d, J=9.0 Hz, 2H), 4.45 (t, J=7.5 Hz, 2H), 1.93 (m, 2H), 1.31 (m, 6H), 0.87 ppm (t, J=6.0 Hz, 3H). ¹³C NMR (125 MHz, CDCl₃) —6=192.43, 141.79, 135.20, 126.91, 121.84, 121.47, 110.26, 77.28, 77.03, 76.77, 43.67, 31.51, 29.13, 26.92, 22.53, 13.97.

### Example 14—Synthesis of the Hydrazone-Linked Tetrahedron 100 from B and Syn-9

Syn-9 (26 mg, 1.83 mmol) and 9-hexyl-9H-carbazole-2,7-dicarbaldehyde (B, 26.0 mg, 19.8 μmol) was dissolved in DCM (4 mL), and the solution was degassed with argon. Next, trifluoroacetic acid (2.27 μL, 29.6 μmol) was added, and the reaction mixture was stirred at room temperature under an argon atmosphere for 24 hours. After 24 hours, the solvent was removed under vacuum leaving the product (the hydrazone-linked tetrahedron 100) (FIG. 8) as an orange solid (35 mg, 100%). ¹H NMR (500 MHz, CDCl₃) δ=11.28 (s, 12H), 8.48 (m, 12H), 8.21 (m, 12H), 7.90 (m, 12H), 7.45 (m, 12H), 6.86 (s, 12H), 4.28 (m, 36H), 3.90 (m, 36H), 3.53-3.21 (m, 204H), 1.91-0.56 (m, 150H).

### Example 15—General Procedure for the Synthesis of the Larger Molecular Tetrahedra

Syn-9 (1 eq) and a dicarbaldehyde (1.5 eq) were dissolved in CH₂Cl₂ (1.5 mM solution) followed by the addition of trifluoroacetic acid (1.0 eq). Next, the reaction mixture was stirred at room temperature for 24 hours. After 24 hours, the solvent was evaporated under reduced pressure. The resulting crude product was dried in vacuo and washed with methanol to provide the hydrazone-linked tetrahedra 11, 12, and 13.

### Example 16—Synthesis of the Hydrazone-Linked Tetrahedron 11 from Syn-9 and [1,1′-Biphenyl]-4,4′-dicarbaldehyde

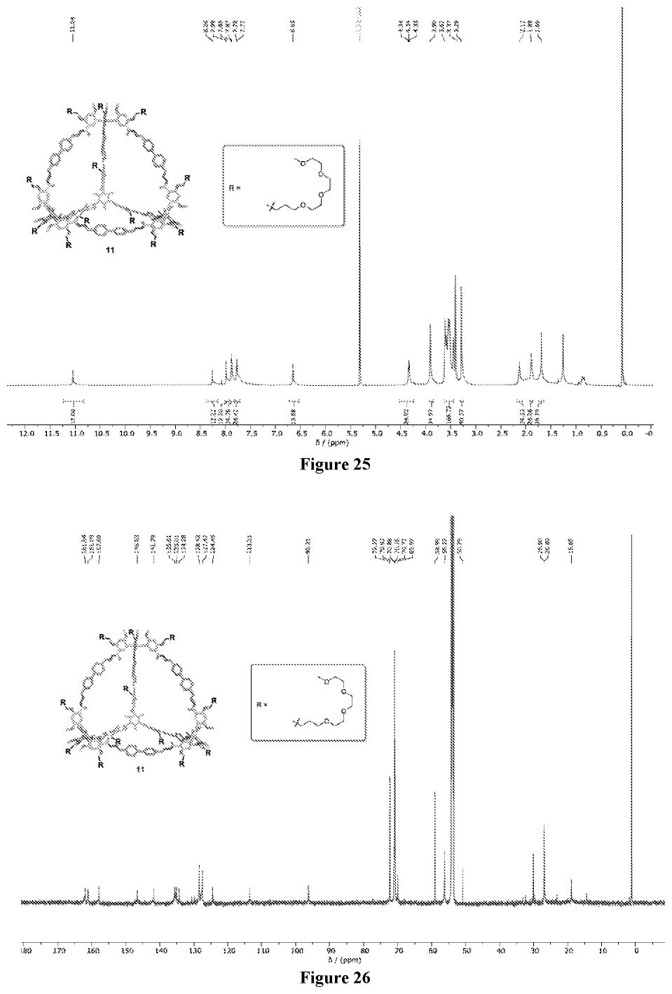

Following the general procedure for the synthesis of the molecular tetrahedra with syn-9 (30.7 mg, 23.3 umol) and [1,1′-biphenyl]-4,4′-dicarbaldehyde (7.4 mg, 35.0 umol), compound 11 was obtained as a yellow solid (32 mg, 88%). ¹H NMR (500 MHz, CD₂Cl₂) δ (ppm); 11.04 (s, 12H), 8.26 (s, 12H), 7.99 (s, 12H), 7.88 to 7.87 (m, 24H), 7.78 to 7.77 (m, 24H) 6.65 (s, 12H), 4.34 (t, J=6.6 Hz, 24H), 3.90 (s, 36H), 3.62-3.47 (m, 168H), 3.29 (s, 36H), 2.12 (m, 24H), 1.89 (m, 24H), 1.69 (s, 36H) (FIG. 25). ¹³C (¹H) NMR (200 MHz, CD₂Cl₂) δ (ppm); 161.94, 161.09, 157.88, 146.63, 141.79, 135.61, 135.01, 134.28, 128.42, 127.47, 124.45, 113.51, 96.21, 72.27, 70.93, 70.88, 70.76, 70.72, 69.97, 58.99, 56.22, 50.79, 26.90, 26.80, 18.85 (FIG. 26). HRMS (ESI+) m/z: [M+5H]⁵⁺ calcd 1262.4349, found 1262.4342.

### Example 17—Synthesis of the Hydrazone-Linked Tetrahedron 12 from Syn-9 and [1,1′: 4′,1″-Terphenyl]-4,4″-dicarbaldehyde

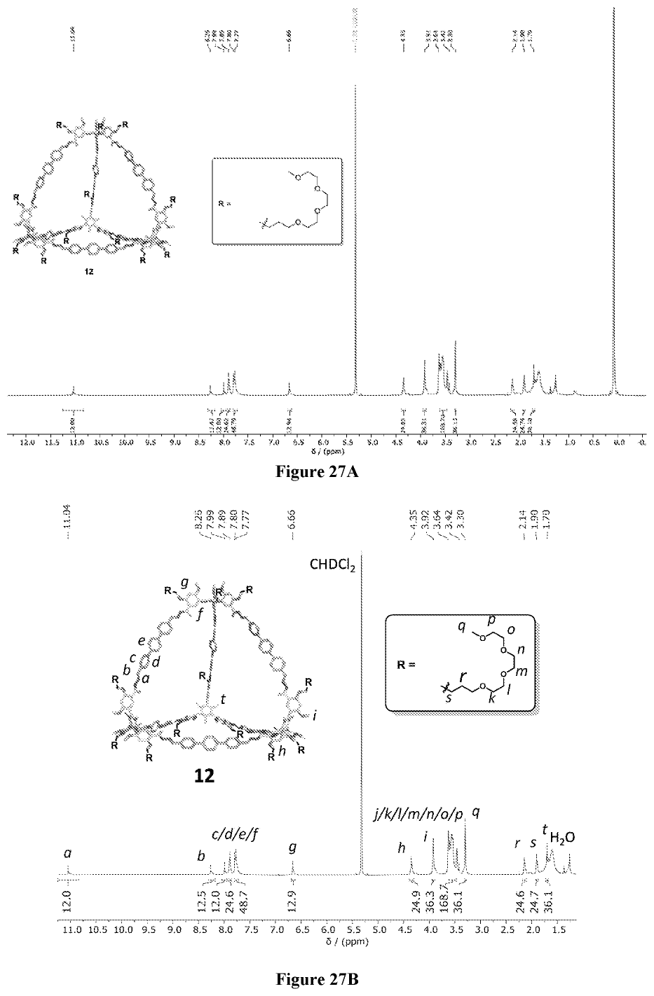

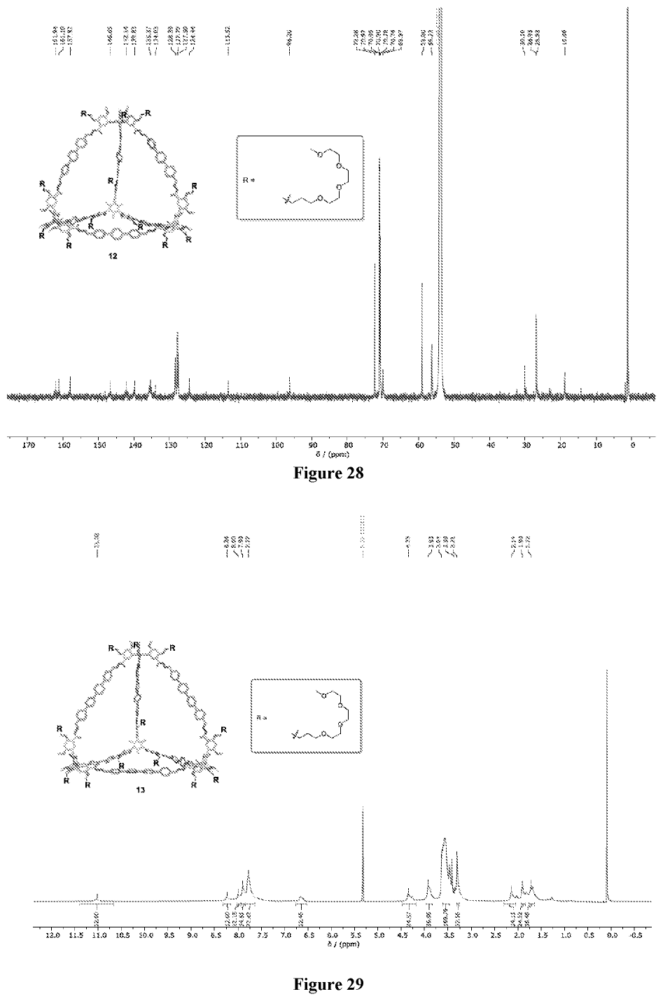

Following the general procedure for the synthesis of the molecular tetrahedra with syn-9 (25.5 mg, 19.4 umol) and [1,1′:4′,1″-terphenyl]-4,4″-dicarbaldehyde (8.33 mg, 29.1 umol), compound 12 was obtained as a yellow solid (30 mg, 92%). ¹H NMR (800 MHz, CD₂Cl₂) δ (ppm); 11.04 (s, 12H), 8.26 (s, 12H), 7.99 (s, 12H), 7.88 to 7.87 (m, 24H), 7.78 to 7.77 (m, 24H) 6.65 (s, 12H), 4.34 (t, J=6.6 Hz, 24H), 3.90 (s, 36H), 3.62-3.47 (m, 168H), 3.29 (s, 36H), 2.12 (m, 24H), 1.89 (m, 24H), 1.69 (s, 36H) (FIGS. 27A-B). ¹³C (¹H) NMR (200 MHz, CD₂Cl₂) δ (ppm); 161.94, 161.10, 157.92, 146.65, 142.14, 139.83, 135.62, 135.37, 135.07, 134.03, 128.39, 127.79, 127.50, 124.44, 113.52, 96.20, 72.28, 70.97, 70.95, 70.90, 70.78, 70.74, 69.97, 59.00, 56.23, 30.10, 26.93, 26.83, 18.86 (FIG. 28). MS (MALDI, DCTB) m/z: [M+Na]⁺ calcd 6786, found 6785.

### Example 18—Synthesis of the Hydrazone-Linked Tetrahedron 13 from Syn-9 and [1,1′:4′,1″:4″,1″-Quaterphenyl]-4,4″-dicarbaldehyde

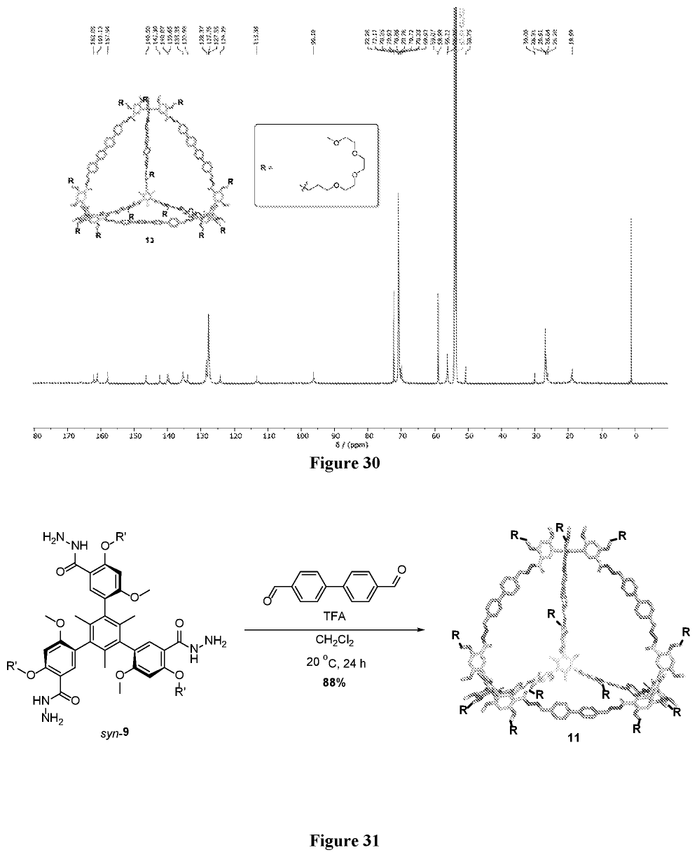

Following the general procedure for the synthesis of the molecular tetrahedra with syn-9 (25.0 mg, 19.0 umol) and [1,1:4′,1:4″,1″-quaterphenyl]-4,4″-dicarbaldehyde (10.3, 28.5 umol), compound 13 was obtained as a yellow solid (29 mg, 86%). ¹H NMR (800 MHz, CD₂Cl₂) δ (ppm); 11.02 (s, 12H), 8.24 (s, 12H), 8.00 (s, 12H), 7.92 to 7.84 (m, 24H), 7.82 to 7.64 (m, 72H) 6.66 (s, 12H), 4.35 (s, 24H), 3.93 (s, 36H), 3.64-3.38 (m, 168H), 3.31 (s, 36H), 2.14 (m, 24H), 1.90 (m, 24H), 1.72 (s, 36H) (FIG. 29). ¹³C (¹H) NMR (200 MHz, CD₂Cl₂) δ (ppm); 162.05, 161.13, 157.94, 146.50, 142.30, 140.07, 139.65, 135.59, 135.35, 135.09, 133.98, 128.37, 127.76, 127.55, 124.29, 113.38, 96.18, 72.26, 72.17, 70.95, 70.93, 70.88, 70.76, 70.72, 70.33, 69.93, 59.07, 58.99, 56.22, 56.16, 50.75, 30.08, 26.91, 26.81, 26.64, 26.20, 18.89 (FIG. 30). MS (MALDI, DCTB) m/z: [M+Na]⁺ calcd 7242, found 7241.

### Example 19—DOSY NMR Spectroscopy

The NMR samples for ¹H DOSY NMR spectroscopy were prepared in CD₂Cl₂ (0.3 mL) at room temperature and recorded with chloroform-d-matched Shigemi NMR tubes. The ¹H DOSY NMR spectra were acquired on a Bruker Avance-III-800 (800 MHz) spectrometer, equipped with a 5 mm QCI Z-gradient cryoprobe, and a Z-axis field gradient module. The DOSY pulse program used was a standard double-stimulated-echo experiment with bipolar gradient pulses and convection compensation. All experiments were acquired at 298 K and DOSY spectra were processed/analyzed using Bruker's TopSpin (version 4.1.1) software. The hydrodynamic radii were estimated using the Stokes-Einstein equation. This equation was s solved for r using values for η from the literature.

$$r = \frac{{k}_{B} \cdot T}{6 \cdot π \cdot η \cdot D} \qquad ( Eq . l )$$

D is the measured diffusion coefficient (m²/s)
k_{B} is Boltzmann constant (1.3806485·10⁻²³ kg m²/s² K)
T is the temperature (K)
r is the solvodynamic radius of the analyte (m)
η is the viscosity of the solvent (0.00041 kg/m s)

### Example 20—Quantum Mechanical Calculations

All structures were optimized with the Jaguar software package (Jaguar, version 11.8, Schrödinger, Inc., New York, NY, 2022; Bochevarov et al., “Jaguar: A High-Performance Quantum Chemistry Software Program with Strengths in Life and Materials Sciences,” J. Quantum Chem. 113:2110-2142 (2013), which are hereby incorporated by reference in their entirety) at the B3LYP/LACVP* level (Becke A. D., “Density-Functional Thermochemistry. III. The Role of Exact Exchange,” J. Chem. Phys. 98:5648-5652 (1993); Lee et al., “Development of the Colle-Salvetti Correlation-Energy Formula Into a Functional of the Electron Density,” Phys. Rev. B 37:785-789 (1988); Vosko et al. “Accurate Spin-Dependent Electron Liquid Correlation Energies for Local Spin Density Calculations: A Critical Analysis,” Can. J. Phys. 58:1200-1211 (1980); Stephens et al., “Ab Initio Calculation of Vibrational Absorption and Circular Dichroism Spectra Using Density Functional Force Fields,” J. Phys. Chem. 98:11623 11627 (1994), which are hereby incorporated by reference in their entirety) of theory. The structures of the molecular cages 10, 11, 12, and 13 were optimized for model compounds with the peripheral —(CH₂)₄(OCH₂CH₂)₃OCH₃ substituents replaced by methyl groups. Due to their very large structures (the model of cage 13, for example, contains over 600 atoms), the structures of the molecular cages were optimized in cartesian coordinates, with loose (Jaguar keyword: iaccg=3) convergence criteria in order to be able to get the geometry optimizations to converge.

To calculate the rotational barrier for conversion of syn-9 to anti-9, a minimum energy dihedral scan was first performed at the B3LYP/LACVP* level, which performed a rotation around one of the rotationally restricted phenyl-phenyl single in syn-9. This initial dihedral scan revealed structures close to the syn-9 to anti-9 transition state and the two structures closest to the transition state (one to either side of the transition state) were then used as the input for an LST transition state search (with an initial LST guess of 0.5) in Jaguar. The transition state search (which was also performed at the B3LYP/LACVP* level) then yielded the geometry of the transition state for the syn-9 to anti-9 atropoisomer conversion. The nature of the transition state was verified by a frequency calculation at the B3LYP/LACVP* level, which showed one imaginary frequency at 32 cm⁻¹.

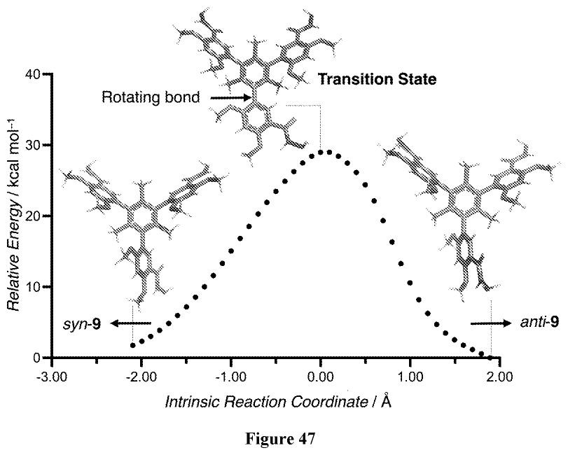

Finally, to ensure that the correct transition state for the syn-9 to anti-9 conversion was found, an intrinsic reaction coordinate scan (FIG. 47) was performed at the B3LYP/LACVP* level, which proceeded energetically downhill in both directions from the transition state to afford anti-9 in the forward direction, and syn-9 in the reverse direction. To calculate the rotational barrier, single point calculations were then performed at the B3LYP/aug-cc-pVDZ level on the optimized structures of syn-9, anti-9, and the syn-9 to anti-9 transition state, which provided an overall activation energy of 28.5 kcal/mol for the syn-9 to anti-9 interconversion.

### Example 21—Results and Discussion of Examples 15-20

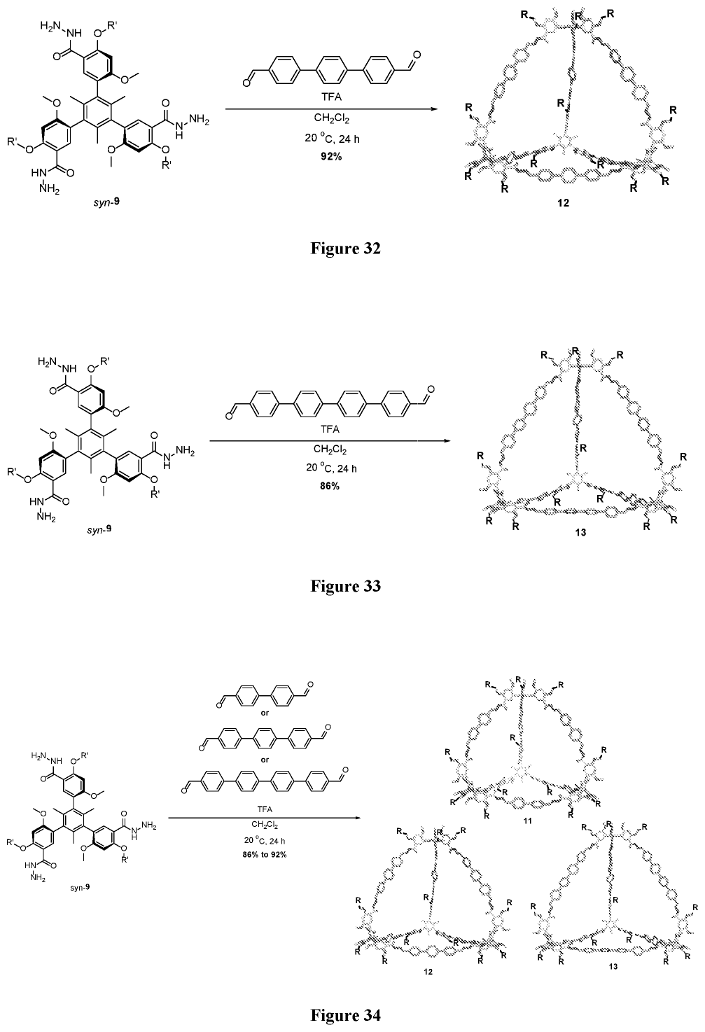

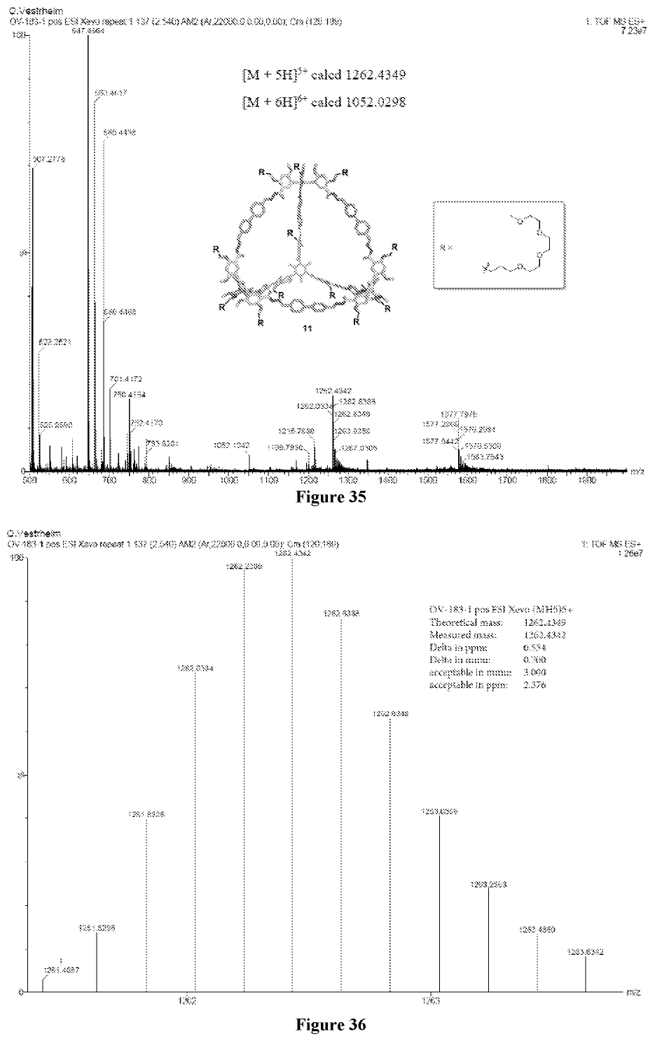

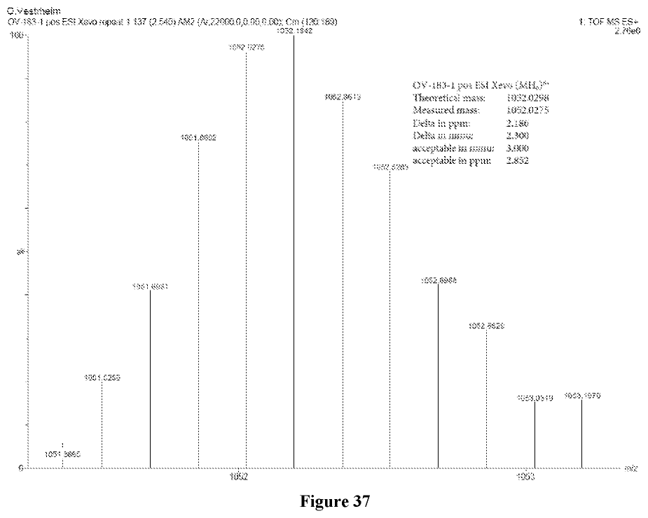

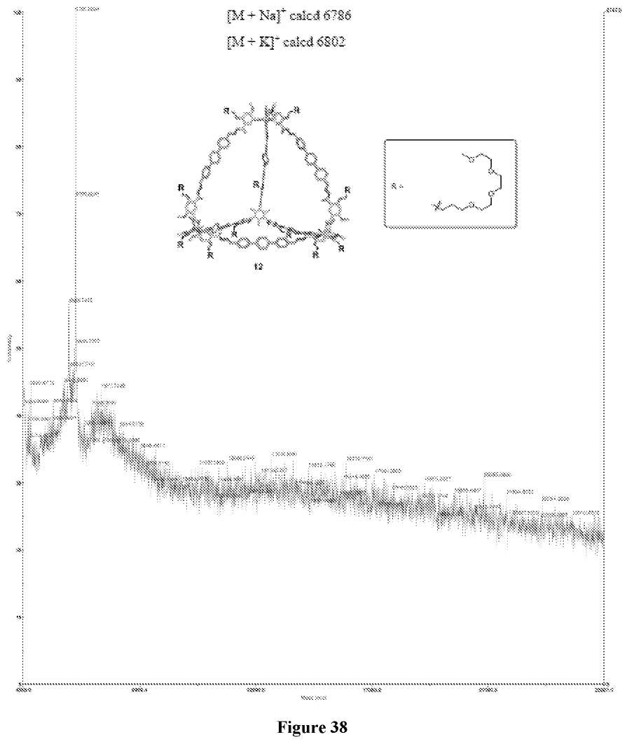

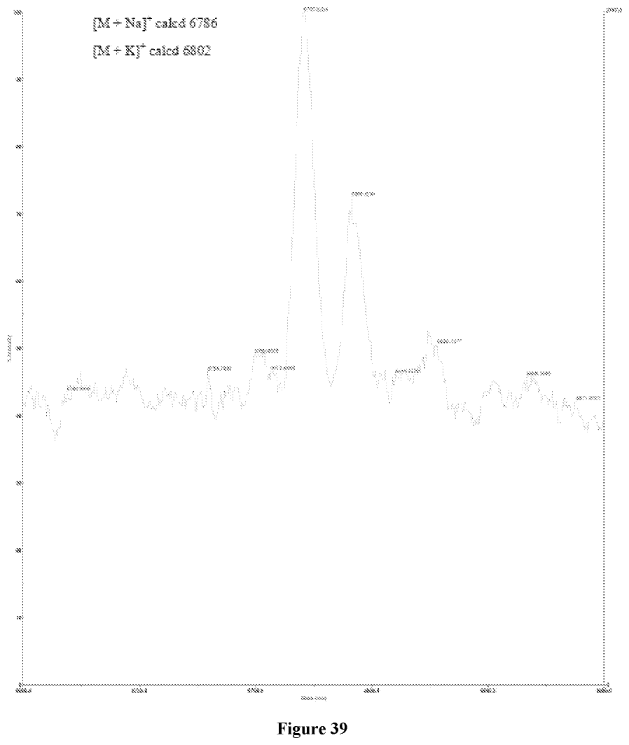

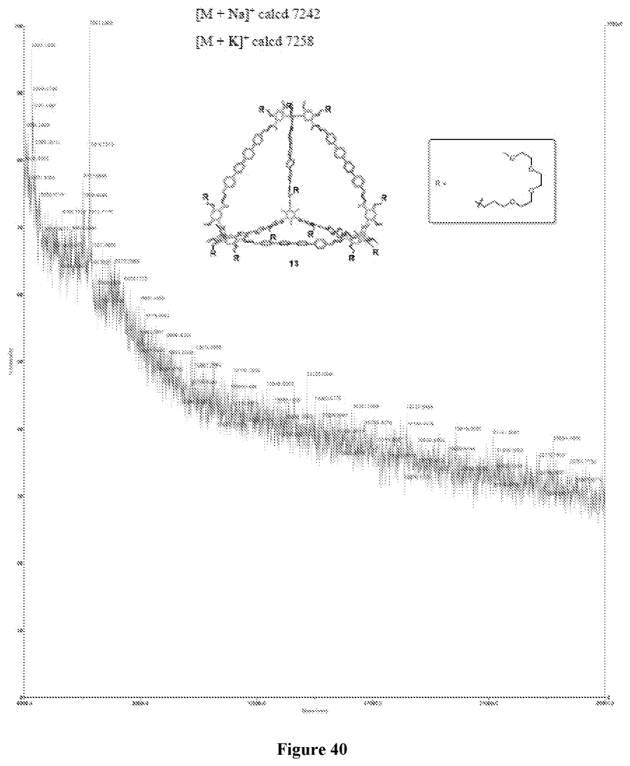

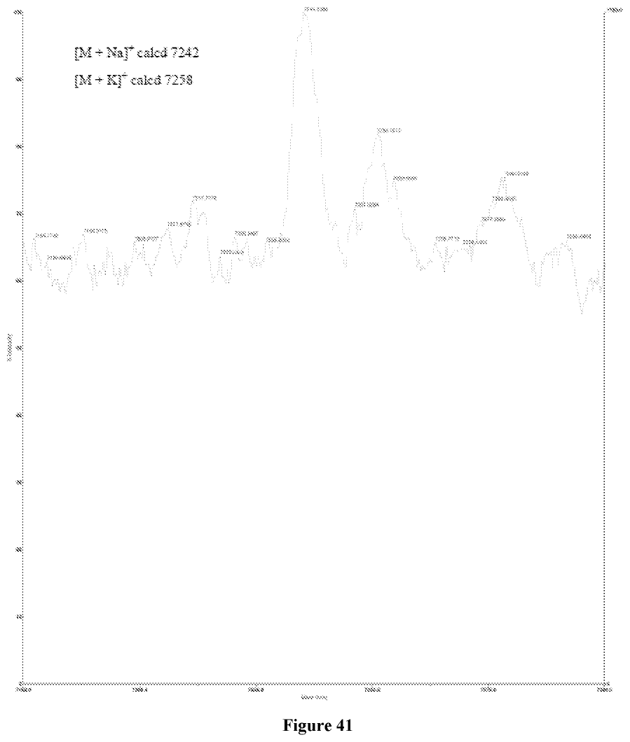

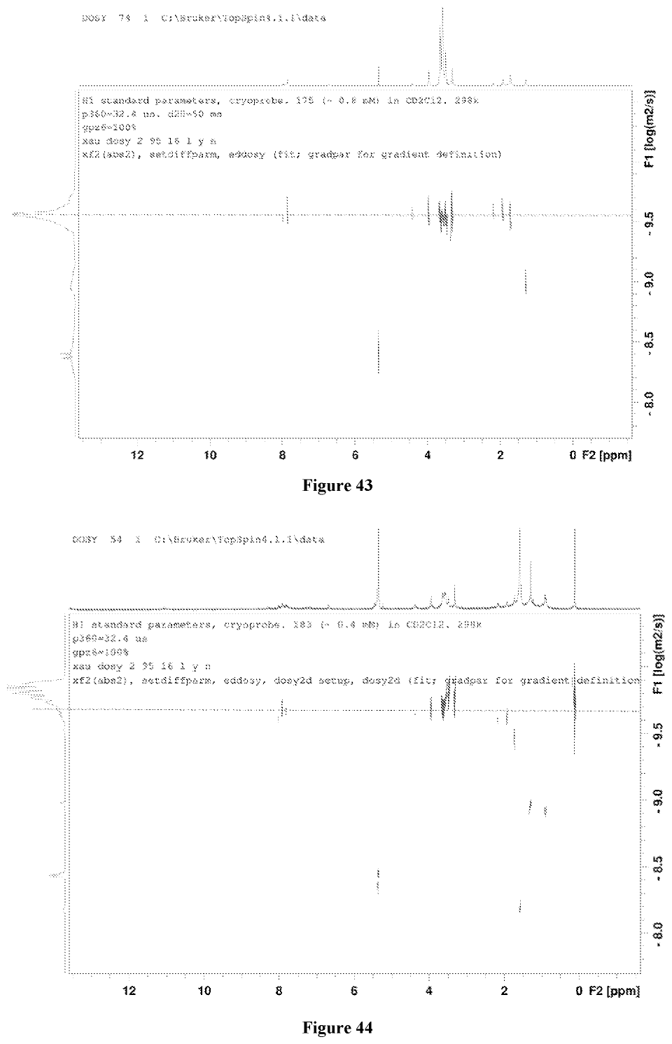

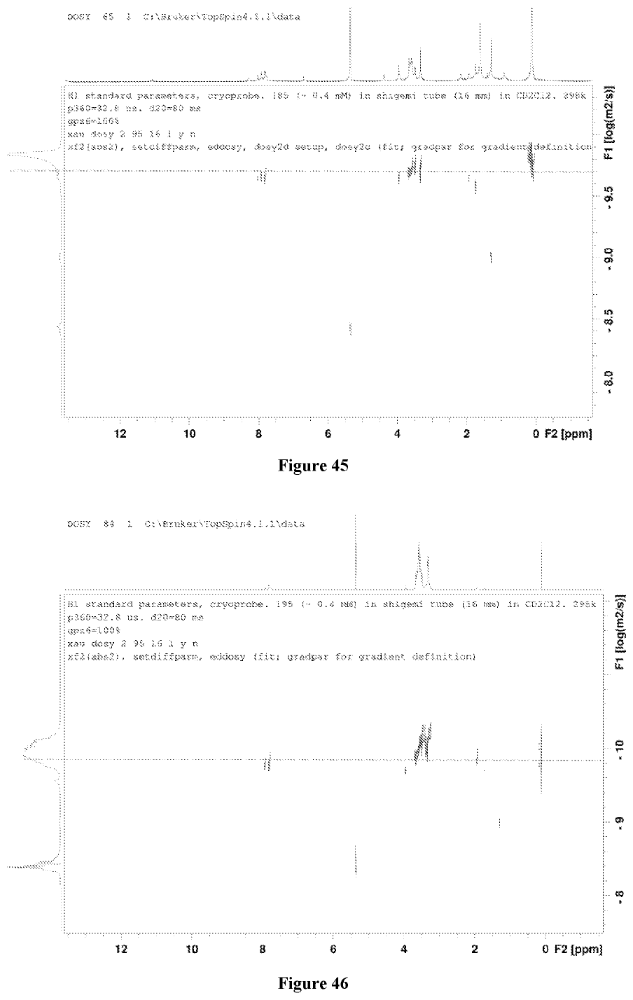

Several molecular cages were prepared from the universal vertex, syn-9 (FIGS. 31-34). These three new cages, compounds 11, 12, and 13 were characterized by ¹H, ¹³C (¹H), and ¹H DOSY NMR spectroscopy (FIGS. 25-30 and 43-46) along with mass spectrometry (FIGS. 35-41). For the biphenyl-linked tetrahedron 11, the [M+5H]⁵⁺ and [M+6H]⁶⁺ ions were observed (FIGS. 35-37) with high resolution electrospray ionization (ESI) mass spectrometry, while the molecular masses of the larger cages, 12 and 13 were verified with matrix-assisted laser desorption ionization (MALDI) mass spectrometry. In the MALDI spectra of 12 and 13 (FIGS. 38-41) both the sodium and potassium ion adducts were observed. Further characterization data was obtained from ¹H DOSY NMR spectroscopy, which provided diffusion coefficients of 2.8×10⁻¹⁰ m² s⁻¹, 2.1×10⁻¹⁰ m² s⁻¹, 2.0×10⁻¹⁰ m² s⁻¹, and 1.4×10⁻¹⁰ m² s⁻¹ for the molecular cages 10, 11, 12, and 13, respectively. Based on the Stokes-Einstein Equation, which assumes a spherical model, these diffusion coefficients translate into approximate solvodynamic radii of 1.9, 2.5, 2.7, and 3.8 nm, respectively, which is in qualitative agreement with the expected sizes of the molecular cages, especially given that the triglyme solubilizing chains and weakly bound solvent molecules are expected to add additional bulk to the molecular cages. Overall, complete elimination of convection from DOSY NMR experiments was very challenging. To bring convection to an absolute minimum, Shigemi NMR tubes (which greatly reduce convection due to a reduced solvent volume) were used and a pulse sequence with convection compensation for all the ¹H DOSY NMR experiments was utilized. Notably, the diffusion coefficient measured for 10 was slightly reduced (2.8×10⁻¹⁰ m² s⁻¹), compared to the diffusion coefficient, which was measured previously (Sharafi et al., “Size-Selective Catalytic Polymer Acylation with a Molecular Tetrahedron,” Chem, 6:1469-1494 (2020), which is hereby incorporated by reference in its entirety) for 10 (5.6×10⁻¹⁰ m² s⁻¹) with a regular 5 mm diameter NMR tube. This finding was consistent with the fact that there was less convection to be expected with a smaller sample volume (i.e. with the Shigemi NMR tube) (Sharafi et al., “Size-Selective Catalytic Polymer Acylation with a Molecular Tetrahedron,” Chem, 6:1469-1494 (2020), which is hereby incorporated by reference in its entirety). Taken together, the relative diffusion coefficients calculated from the ¹H DOSY NMR spectra for the three new cages 11, 12, and 13 as well as for our original tetrahedron 10 clearly indicate an increasing solvdynamic radius from the smallest to the largest cage (Table 1), which is in qualitative agreement with the DFT-optimized structures of the molecular cages.

TABLE 1
Diffusion Coefficients Measured by ¹H DOSY NMR Spectroscopy
in CD₂Cl₂ for the Molecular Cages 10, 11, 12, and 13

| Compound | T [K] | Log D [log(m²/s) | D · 10⁻¹⁰ [m²/s] | r (nm) |
|---|---|---|---|---|
| 10 | 298 | −9.56 | 2.75 | 1.9 |
| 11 | 298 | −9.67 | 2.14 | 2.5 |
| 12 | 298 | −9.70 | 2.00 | 2.7 |
| 13 | 298 | −9.85 | 1.41 | 3.8 |

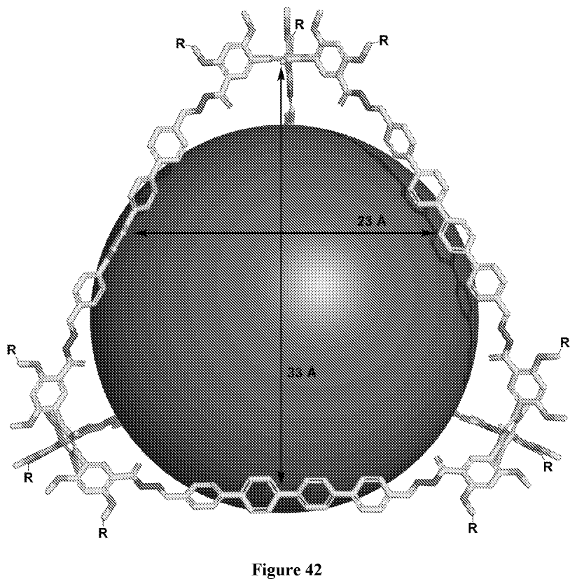

These new structures (especially compound 13, which contains 6p-quarterphenyl linkers), represent some of the largest tetrahedral porous organic cages created to date (Sharafi et al., “Size-Selective Catalytic Polymer Acylation with a Molecular Tetrahedron,” Chem, 6:1469 1494 (2020); Chakraborty et al., “Recent Trends in Organic Cage Synthesis: Push Towards Water-Soluble Organic Cages,” Chem. Commun. 58:5558-5573 (2022); Monta-Gonzalez et al., “Purely Covalent Molecular Cages and Containers for Guest Encapsulation,” Chem. Rev. 122:13636-13708 (2022); Betancourth et al., “Versatility of the Amino Group in Hydrazone-based Molecular and Supramolecular Systems,” Eur. J. Org. Chem. 2022:e202200228 (2022); Mastalerz M., “Porous Shape-Persistent Organic Cage Compounds of Different Size, Geometry, and Function,” Accounts of Chemical Research 51:2411-2422 (2018), which are hereby incorporated by reference in their entirety). The largest cage (compound 13) contains a cavity volume of approximately 17 nm³ and an internal height of about 33 Å (FIG. 42). Notably, the compound 13 was synthesized with only a single chromatographic purification step, which provides a practical path to gram-scale quantities of the common vertex syn-9 (FIG. 7).

The rotational barrier was calculated with density functional theory (DFT). The structure of the transition state for the syn-9 to anti-9 conversion was first optimized at the B3LYP/LACVP* level. Next, an intrinsic reaction coordinate (IRC) coordinate scan (FIG. 47) was performed, starting from the transition state, and then optimized the geometries of syn-9 and anti-9. Finally, the activation energy for the syn-9 to anti-9 interconversion was calculated at the B3LYP/aug-cc-PVDZ/B3LYP/LACVP* level which provided a value of 28.5 kcal/mol for the activation energy (with zero-point energy corrections calculated at the B3LYP/LACVP* level). At the same level of theory, the optimized structure of anti-9 was found to lie 2.3 kcal/mol lower in energy than syn-9. The relatively high rotational barrier of nearly 29 kcal/mol explains why the syn-vertex is configurationally stable during the gram-scale synthetic protocol.

While the synthesis of tetrahedral cages and cages with large pore openings in general is often complicated by intercatenation (Shen et al., “Dynamic Covalent Self-Assembly Based on Oxime Condensation,” Angewandte Chemie International Edition 57: 16486-16490 (2018); Li, et al., “Quantitative Self-Assembly of a Purely Organic Three-Dimensional Catenane in Water,” Nature Chemistry 7:1003-1008 (2015); Lin et al., “Multi component Assembly of Cavitand-Based Polyacylhydrazone Nanocapsules,” Chemistry—A European Journal 17:9395-9405 (2011), which are hereby incorporated by reference in their entirety), intercatenation was not observed in the synthesis described in the present disclosure. The glyme-derived solubilizing chains attached to all the vertices stabilized the cages in their monomeric forms in solution, which helped to prevent intercatenation. With the successful formation of the larger cavities, access to a much greater selection of potential targets for selective recognition and catalysis inside the tetrahedral cages was obtained, since the larger cages contain cavities large enough for endohedral functionalization. In addition, as a tetrahedral geometry provided the largest openings of any polyhedral shape (Mahata et al., “Giant Electroactive M4L6 Tetrahedral Host Self-Assembled with Fe(II) Vertices and Perylene Bisimide Dye Edges,” Journal of the American Chemical Society 135:15656-15661 (2013), which is hereby incorporated by reference in its entirety), the cages described in the present disclosure will provide the possibility of larger polymers with bulky side chains to gain access to the cavity of the cage, with the potential for processive catalytic functionalization (Sharafi et al., “Size-Selective Catalytic Polymer Acylation with a Molecular Tetrahedron,” Chem, 6:1469-1494 (2020), which is hereby incorporated by reference in its entirety).

In summary, a facile new synthesis of a covalent, hydrazone-linked molecular tetrahedron described in the present disclosure allowed for production of the catalytically-active tetrahedron (Sharafi et al., “Size-Selective Catalytic Polymer Acylation with a Molecular Tetrahedron,” Chem, 6:1469-1494 (2020), which is hereby incorporated by reference in its entirety) for the first time on a multigram scale in a single batch. This new procedure requires a minimal amount of purification as it relies heavily on precipitation in water rendering the synthesis much more practical for large scale. In the nine synthetic steps performed, only one compound required purification by column chromatography. Furthermore, this new synthesis provides access to a common late-stage intermediate, which will allow to expand the family of tetrahedral molecular cages in the future to tune the peripheral functional groups for selective recognition and catalysis. Using this method, three new larger cages were synthesized. These cages represent some of the largest covalent molecular tetrahedra reported to date (Monta-Gonzalez et al., “Purely Covalent Molecular Cages and Containers for Guest Encapsulation,” Chem. Rev. 122:13636-13708 (2022); Yang et al., “Porous Organic Cages,” Chem. Rev. 123:4602-4634 (2023), which are hereby incorporated by reference in their entirety).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A nanocage of Formula (I): 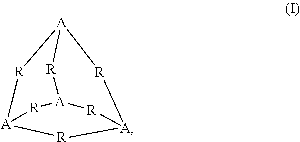
wherein
each A is independently selected and has the formula 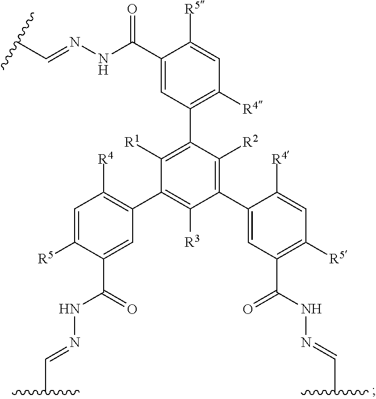
is the point of attachment of A to R;
each R is independently selected and has the formula 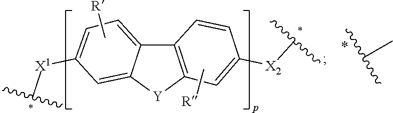
indicates the point of attachment of R to A;
R′ is H or C₁₋₂₀ alkyl;
R″ is H or C₁₋₂₀ alkyl;
R¹, R², and R³ are each independently selected from the group consisting of H, halogen, —OH, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated Ciao alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated Ciao alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁴, R^{4′}, and R^{4″} are each independently selected from the group consisting of H, halogen, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OH, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁵, R^{5′}, and R^{5″} are each independently selected from the group consisting of H, halogen, —OH, —O(CH₂)ₙ—(OCH₂CH₂)ₘ—OC₁₋₆ alkyl, —O(CH₂)ₙ—(OCH₂CH₂)ₘ—OH, (OCH₂CH₂)ₘ—OC₁₋₆ alkyl, (OCH₂CH₂)ₘ—OH, aryl, heteroaryl, heterocyclyl, —OC₁₋₂₀ alkyl, —O-perfluorinated C₁₋₂₀ alkyl, —Oaryl, and —NR¹⁰R¹¹ wherein R¹⁰ and R¹¹ are each independently selected from the group consisting of H, C₁₋₆ alkyl, C₂₋₆ alkenyl, C₂₋₆ alkynyl, aryl, heteroarylalkyl, heterocyclylalkyl, and arylalkyl, wherein each C₁₋₆ alkyl, C₂₋₆ alkenyl, C₂₋₆ alkynyl, aryl, heteroarylalkyl, heterocyclylalkyl, and arylalkyl can be optionally substituted from 1 to 3 times with a substituent selected from the group consisting of H, C₁₋₆ alkyl, heteroarylalkyl, heterocyclylalkyl, and arylalkyl;
X¹ and X² are absent or are independently selected from the group consisting of C₁₋₆ alkylene, C₃₋₈ cycloalkylene, and arylene, wherein C₁₋₆ alkylene, C₃₋₈ cycloalkylene, and arylene can be optionally substituted from 1 to 3 times with H or C₁₋₆ alkyl;
Y is independently selected from the group consisting of —N(R⁶)—, —S—, —O—, —C(R⁶)(R^{6′})—, and —P(R⁶)—, wherein R⁶ and R^{6′} are each independently selected at each occurrence from the group consisting of H, C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl, wherein C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl can be substituted from 1 to 3 times with a substituent selected from the group consisting of —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated Ciao alkyl, —COOaryl, —CONHC₁₋₂₀ alkyl, —CONHC₂₋₂₀ alkenyl, —CONHC₂₋₂₀ alkynyl, —CONH perfluorinated Ciao alkyl, —CONH-aryl, —CONH-heteroaryl, and CONH-heterocycles;
p is 1 to 5;
n is 1 to 10; and
m is 1 to 50.

2. (canceled)

3. (canceled)

4. (canceled)

5. The nanocage of claim 1, wherein R¹, R², and R³ are Me, R⁴ is —OMe, R⁵ is —O(CH₂)₄—(OCH₂CH₂)₃—OMe, and R⁶ is —C₆H₁₃.

6. A process for preparation of a nanocage of Formula (I): 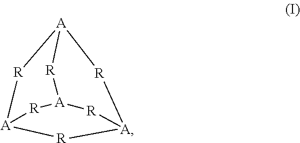
each A is independently selected and has the formula 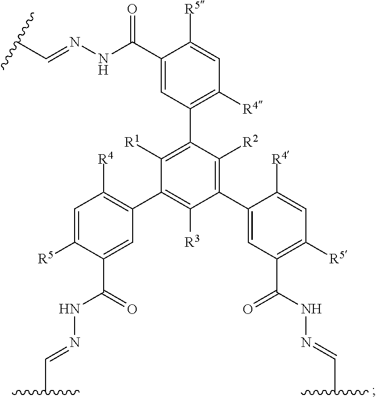
is the point of attachment of A to R;
each R is independently selected and has the formula 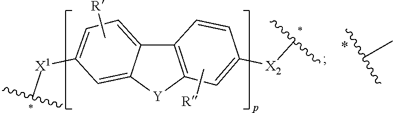
indicates the point of attachment of R to A;
R′ is H or C₁₋₂₀ alkyl;
R″ is H or C₁₋₂₀ alkyl;
R¹, R², and R³ are each independently selected from the group consisting of H, halogen, —OH, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated Ciao alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated Ciao alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁴, R^{4′}, and R^{4″} are each independently selected from the group consisting of H, halogen, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OH, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁵, R^{5′}, and R^{5″} are each independently selected from the group consisting of H, halogen, OH, —O(CH₂)ₙ—(OCH₂CH₂)ₘ—OC₁₋₆ alkyl, —O(CH₂)ₙ—(OCH₂CH₂)ₘ—OH, (OCH₂CH₂)ₘ—OC₁₋₆ alkyl, (OCH₂CH₂)ₘ—OH, aryl, heteroaryl, heterocyclyl, —OC₁₋₂₀ alkyl, —O-perfluorinated C₁₋₂₀ alkyl, —Oaryl, and —NR¹⁰R¹¹ wherein R¹⁰ and R¹¹ are each independently selected from the group consisting of H, C₁₋₆ alkyl, C₂₋₆ alkenyl, C₂₋₆ alkynyl, aryl, heteroarylalkyl, heterocyclylalkyl, and arylalkyl, wherein each C₁₋₆ alkyl, C₂₋₆ alkenyl, C₂₋₆ alkynyl, aryl, heteroarylalkyl, heterocyclylalkyl, and arylalkyl can be optionally substituted from 1 to 3 times with a substituent selected from the group consisting of H, C₁₋₆ alkyl, heteroarylalkyl, heterocyclylalkyl, and arylalkyl;
X¹ and X² are absent or are independently selected from the group consisting of C₁₋₆ alkylene, C₃₋₈ cycloalkylene, and arylene, wherein C₁₋₆ alkylene, C₃₋₈ cycloalkylene, and arylene can be optionally substituted from 1 to 3 times with H or C₁₋₆ alkyl;
Y is independently selected from the group consisting of —N(R⁶)—, —S—, —O—, —C(R⁶)(R^{6′})—, and —P(R⁶)—, wherein R⁶ and R^{6′} are each independently selected at each occurrence from the group consisting of H, C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl, wherein C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl can be substituted from 1 to 3 times with a substituent selected from the group consisting of —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated Ciao alkyl, —COOaryl, —CONHC₁₋₂₀ alkyl, —CONHC₂₋₂₀ alkenyl, —CONHC₂₋₂₀ alkynyl, —CONH perfluorinated Ciao alkyl, —CONH-aryl, —CONH-heteroaryl, and CONH-heterocyclyl;
p is 1 to 5;
n is 1 to 10; and
m is 1 to 50, said process comprising:
providing one or more compounds of Formula (II) having the structure: 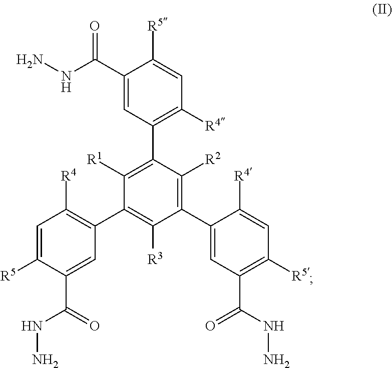
and
forming the nanocage of Formula (I) from the one or more compounds of Formula (II), wherein said forming the nanocage of Formula (I) comprises:
reacting the one or more compounds of Formula (II) with one or more compounds of Formula (III): 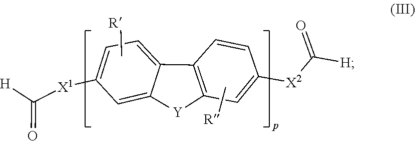
to produce the nanocage of Formula (I).

7. (canceled)

8. The process according to claim 6, wherein the compound of Formula (III) has the following structure:

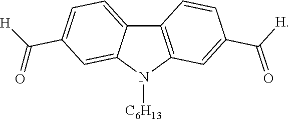

9. The process according to claim 6 further comprising:
reacting one or more compounds of Formula (IV) having the structure: 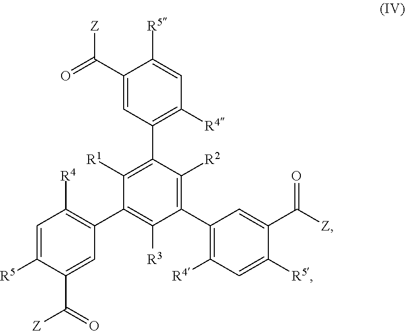
wherein
Z is —O—C₁₋₆ alkyl,
with a hydrazine source to form the one or more compounds of Formula (II).

10. (canceled)

11. The process according to claim 9 further comprising:
reacting one or more compounds of Formula (V) having the structure: 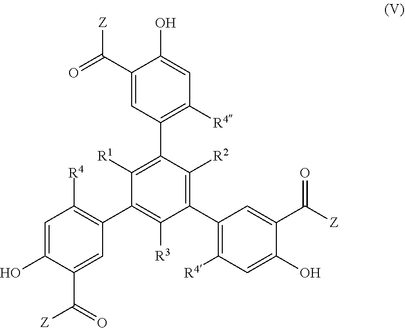
with one or more compounds of Formula (VI):
LG-R¹² (VI),
wherein LG is a suitable leaving group and R¹² is selected from a group consisting of —(CH₂)ₙ—(OCH₂CH₂)ₘ—OC₁₋₆ alkyl, —(CH₂)ₙ—(OCH₂CH₂)ₘ—OH, —CH₂CH₂—(OCH₂CH₂)ₘ₋₁—OC₁₋₆ alkyl, —CH₂CH₂—(OCH₂CH₂)ₘ₋₁—OH, —C₁₋₂₀ alkyl, -perfluorinated C₁₋₂₀ alkyl, and -aryl to form the one or more compounds of Formula (IV).

12. (canceled)

13. The process according to claim 11, wherein the one or more compounds of Formula (VI) has the formula:
Hal-(CH₂)ₙ—(OCH₂CH₂)ₘ—OC₁₋₆ alkyl,
wherein Hal is Cl or Br.

14. The process according to claim 11 further comprising:
reacting one or more compounds of Formula (VII) having the structure: 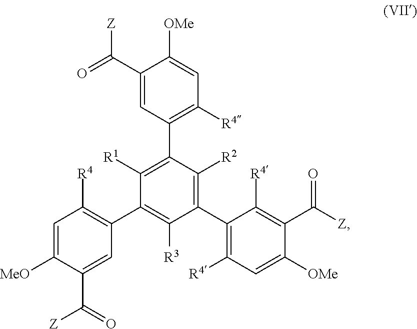
with a compound of Formula (VIII)
BCl₃ (VIII)
to form the one or more compounds of Formula (V).

15. (canceled)

16. The process according to claim 14 further comprising:
esterifying one or more compounds of Formula (IX) having the structure: 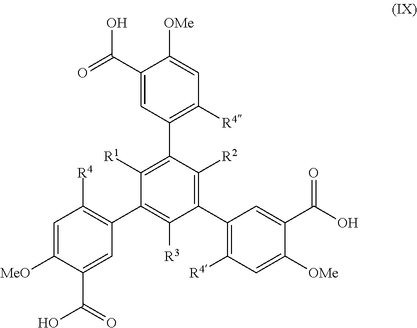
with MeOH to form the one or more compounds of Formula (VII).

17. (canceled)

18. The process according to claim 16 further comprising:
reacting one or more compounds of Formula (X) having the structure: 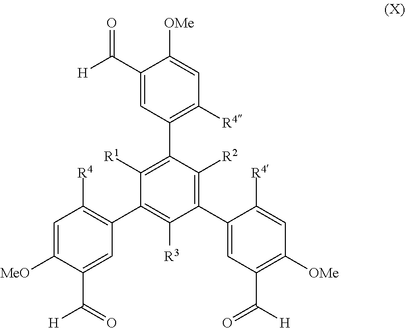
with an oxidizing agent to form the one or more compounds of Formula (IX).

19. (canceled)

20. (canceled)

21. The process according to claim 18 further comprising:
reacting one or more compounds of Formula (XI) having the structure: 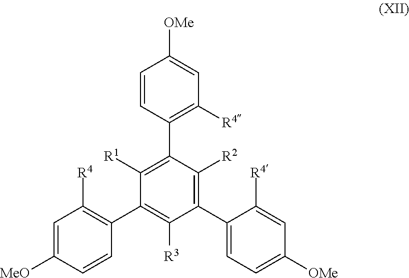
with a formylating agent to form the one or more compounds of Formula (X).

22. (canceled)

23. (canceled)

24. The process according to claim 21 further comprising:
reacting one or more compounds of Formula (XII) having the structure: 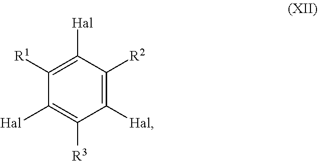
wherein Hal is halogen, with one or more compounds of Formula (XIII): 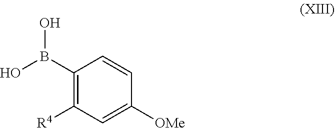
to form the one or more compounds of Formula (XI).

25. (canceled)

26. The process according to claim 24 further comprising:
reacting one or more compounds of Formula (XIV) having the structure: 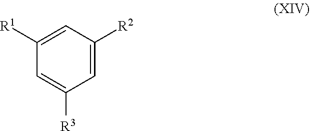
with a halogenating agent to form the one or more compounds of Formula (XII).

27. (canceled)

28. (canceled)

29. A process for preparation of a nanocage of Formula (I′): 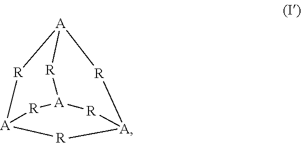
wherein
each A is independently selected and has the formula 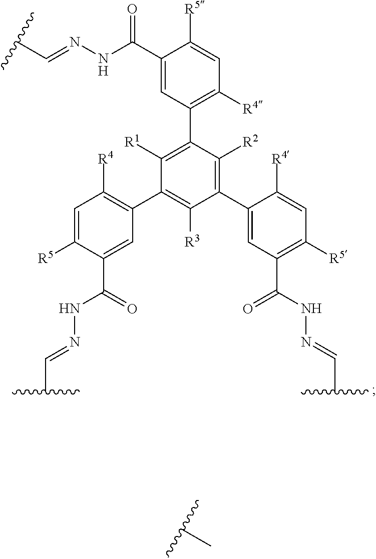
is the point of attachment of A to R;
each R is independently selected and has the formula 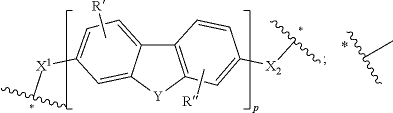
indicates the point of attachment of R to A;
R′ is H or C₁₋₂₀ alkyl;
R″ is H or C₁₋₂₀ alkyl;
R¹, R², and R³ are each independently selected from the group consisting of H, halogen, —OH, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁴, R^{4′}, and R^{4″} are each independently selected from the group consisting of H, halogen, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OH, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁵, R^{5′}, and R^{5″} are each independently selected from the group consisting of H, halogen, —OH, —O(CH₂)ₙ—(OCH₂CH₂)ₘ—OC₁₋₆ alkyl, —O(CH₂)ₙ—(OCH₂CH₂)ₘ—OH, (OCH₂CH₂)ₘ—OC₁₋₆ alkyl, (OCH₂CH₂)ₘ—OH, aryl, heteroaryl, heterocyclyl, —OC₁₋₂₀ alkyl, —O-perfluorinated C₁₋₂₀ alkyl, —Oaryl, and —NR¹⁰R¹¹ wherein R¹⁰ and R¹¹ are each independently selected from the group consisting of H, C₁₋₆ alkyl, C₂₋₆ alkenyl, C₂₋₆ alkynyl, aryl, heteroarylalkyl, heterocyclylalkyl, and arylalkyl, wherein each C₁₋₆ alkyl, C₂₋₆ alkenyl, C₂₋₆ alkynyl, aryl, heteroarylalkyl, heterocyclylalkyl, and arylalkyl can be optionally substituted from 1 to 3 times with a substituent selected from the group consisting of H, C₁₋₆ alkyl, heteroarylalkyl, heterocyclylalkyl, and arylalkyl;
X¹ and X² are absent or are independently selected from the group consisting of C₁₋₆ alkylene, C₃₋₈ cycloalkylene, and arylene, wherein C₁₋₆ alkylene, C₃₋₈ cycloalkylene, and arylene can be optionally substituted from 1 to 3 times with H or C₁₋₆ alkyl;
Y is independently selected from the group consisting of —N(R^{6′})—, —S—, —O—, —C(R^{6′})(R^{6″})—, and —P(R^{6′})—, wherein R^{6′} and R^{6″} are each independently selected at each occurrence from the group consisting of H, C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl, wherein C₁₋₂₀ alkyl, C₂₋₂₀ alkenyl, C₂₋₂₀ alkynyl, C₃₋₈ cycloalkyl, perfluorinated C₁₋₂₀ alkyl, awl, heteroaryl, and heterocyclyl can be substituted from 1 to 3 times with a substituent selected from the group consisting of —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl;
p is 1 to 5;
n is 1 to 10; and
m is 1 to 50, said process comprising:
providing one or more compounds of Formula (V′) having the structure: 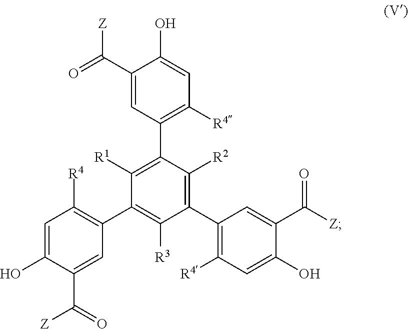
and
forming the nanocage of Formula (I′) from the one or more compounds of Formula (V′).

30. The process according to claim 29, wherein said forming the nanocage of Formula (I′) comprises:
reacting the one or more compounds of Formula (V′) with one or more compounds of Formula (VI′): 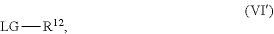
wherein LG is a suitable leaving group; and
R¹² is selected from a group consisting of (CH₂)ₙ—(OCH₂CH₂)ₘ—OC₁₋₆ alkyl, —(CH₂)ₙ—(OCH₂CH₂)ₘ—OH, CH₂CH₂—(OCH₂CH₂)ₘ₋₁—OC₁₋₆ alkyl, CH₂CH₂—(OCH₂CH₂)ₘ₋₁—OH, C₁₋₂₀ alkyl, -perfluorinated C₁₋₂₀ alkyl, and -aryl;
to produce the one or more compounds of Formula (IV′): 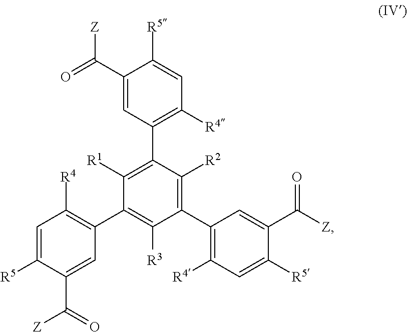
wherein Z is —O—C₁₋₆ alkyl;
reacting the one or more compounds of Formula (IV′) with a hydrazine source to produce the one or more compounds of Formula (II′): 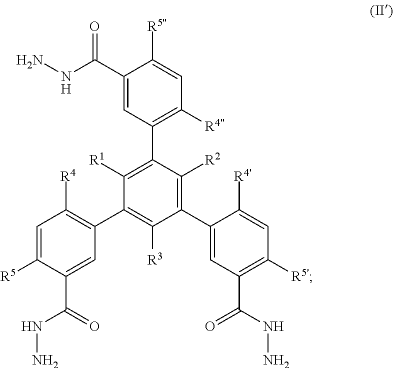
reacting one or more compounds of Formula (II′) with one or more compounds of Formula (III′A): 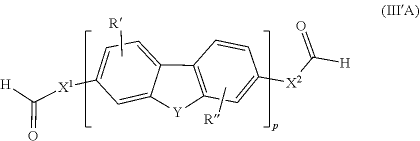
to produce the nanocage of Formula (I′).

31. The process according to claim 29, wherein the compound of Formula (III′A) has the following structure: 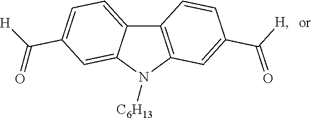
the one or more compounds of Formula (VI′) has the formula:
Hal-(CH₂)ₙ—(OCH₂CH₂)ₘ—OC₁₋₆ alkyl, wherein Hal is Cl or Br.

32. (canceled)

33. (canceled)

34. The process according to claim 29 further comprising:
reacting one or more compounds of Formula (VII′) having the structure: 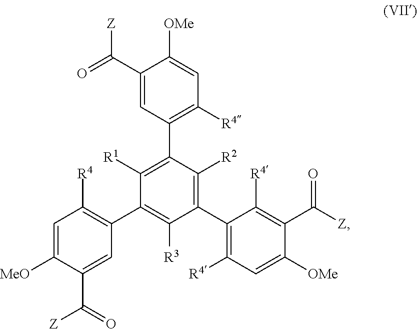
with a compound of Formula (VIII′)
BCl₃ (VIII′)
to form the one or more compounds of Formula (V′).

35. (canceled)

36. The process according to claim 34 further comprising:
esterifying one or more compounds of Formula (IX′) having the structure: 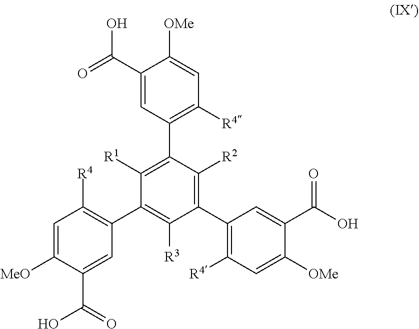
with MeOH to form the one or more compounds of Formula (VII′).

37. (canceled)

38. The process according to claim 36 further comprising:
reacting one or more compounds of Formula (X′) having the structure: 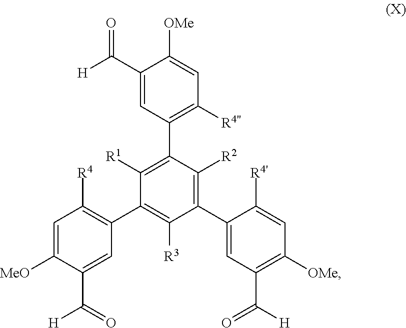
with an oxidizing agent to form the one or more compounds of Formula (IX′).

39. (canceled)

40. (canceled)

41. The process according to claim 38 further comprising:
reacting one or more compounds of Formula (XI′) having the structure: 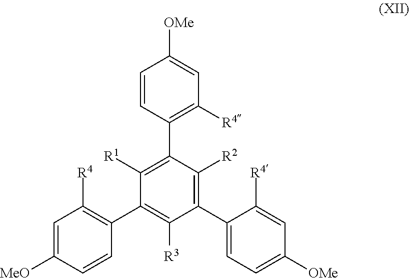
with a formylating agent to form the one or more compounds of Formula (X′).

42. (canceled)

43. (canceled)

44. The process according to claim 41 further comprising:
reacting one or more compounds of Formula (XII′) having the structure: 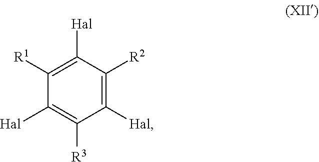
wherein Hal is halogen, with one or more compounds of Formula (XIII′): 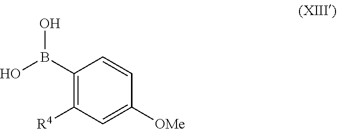
to form the one or more compounds of Formula (XI′).

45. (canceled)

46. The process according to claim 44 further comprising:
reacting one or more compounds of Formula (XIV′) having the structure: 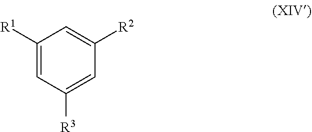
with a halogenating agent to form the one or more compounds of Formula (XII′).

47. (canceled)

48. (canceled)

49. A method for detecting an analyte in a fluid comprising:
providing a sensor comprising a nanocage of Formula (I): 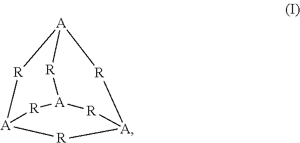
wherein
each A is independently selected and has the formula 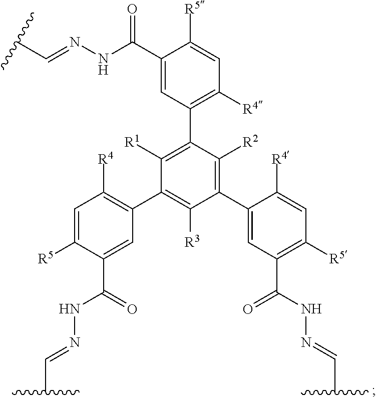
is the point of attachment of A to R;
each R is independently selected and has the formula 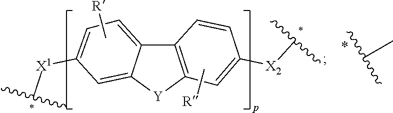
indicates the point of attachment of R to A;
R′ is H or C₁₋₂₀ alkyl;
R″ is H or C₁₋₂₀ alkyl;
R¹, R², and R³ are each independently selected from the group consisting of H, halogen, —OH, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated Ciao alkyl, —Oaryl, —COOC₁₋₂₀ alkyl, —COO perfluorinated Ciao alkyl, —COOaryl, —CONHC₁₋₂₀ alkyl, —CONHC₂₋₂₀ alkenyl, —CONHC₂₋₂₀ alkynyl, —CONH perfluorinated Ciao alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁴, R^{4′}, and R^{4″} are each independently selected from the group consisting of H, halogen, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OH, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁵, R^{5′}, and R^{5″} are each independently selected from the group consisting of H, halogen, —OH, —O(CH₂)ₙ—(OCH₂CH₂)ₘ—OC₁₋₆ alkyl, —O(CH₂)ₙ—(OCH₂CH₂)ₘ—OH, (OCH₂CH₂)ₘ—OC₁₋₆ alkyl, (OCH₂CH₂)ₘ—OH, aryl, heteroaryl, heterocyclyl, —OC₁₋₂₀ alkyl, —O-perfluorinated C₁₋₂₀ alkyl, —Oaryl, and —NR¹⁰R¹¹ wherein R¹⁰ and R¹¹ are each independently selected from the group consisting of H, C₁₋₆ alkyl, C₂₋₆ alkenyl, C₂₋₆ alkynyl, aryl, heteroarylalkyl, heterocyclylalkyl, and arylalkyl, wherein each C₁₋₆ alkyl, C₂₋₆ alkenyl, C₂₋₆ alkynyl, aryl, heteroarylalkyl, heterocyclylalkyl, and arylalkyl can be optionally substituted from 1 to 3 times with a substituent selected from the group consisting of H, C₁₋₆ alkyl, heteroarylalkyl, heterocyclylalkyl, and arylalkyl;
X¹ and X² are absent or are independently selected from the group consisting of C₁₋₆ alkylene, C₃₋₈ cycloalkylene, and arylene, wherein C₁₋₆ alkylene, C₃₋₈ cycloalkylene, and arylene can be optionally substituted from 1 to 3 times with H or C₁₋₆ alkyl;
Y is independently selected from the group consisting of —N(R⁶)—, —S—, —O—, —C(R⁶)(R^{6′})—, and —P(R⁶)—, wherein R⁶ and R^{6′} are each independently selected at each occurrence from the group consisting of H, C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl, wherein C₁₋₂₀ alkyl, C₂₋₂₀ alkenyl, C₂₋₂₀ alkynyl, C₃₋₈ cycloalkyl, perfluorinated C₁₋₂₀ alkyl, awl, heteroaryl, and heterocyclyl can be substituted from 1 to 3 times with a substituent selected from the group consisting of —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated Ciao alkyl, —COOaryl, —CONHC₁₋₂₀ alkyl, —CONHC₂₋₂₀ alkenyl, —CONHC₂₋₂₀ alkynyl, —CONH perfluorinated Ciao alkyl, —CONH-aryl, —CONH-heteroaryl, and CONH-heterocyclyl;
p is 1 to 5;
n is 1 to 10; and
m is 1 to 50;
providing a fluid containing an analyte; and
contacting a fluid containing the analyte with the sensor to capture the analyte in the nanocage and detect the analyte in the fluid.

50. The method according to claim 49, wherein the sensor further comprises:
a substrate having a surface with a layer of the nanocage of Formula (I) covering at least 5% of the surface.

51. The method according to claim 49, wherein the sensor further comprises:
a substrate having a surface with a layer of the nanocage of Formula (I) covering at least 10% of the surface.

52. The method according to claim 49 further comprising:
providing a signal generator operatively associated with said sensor, said method further comprising:
producing a signal with the signal generator when said analyte is captured by said sensor.

53. The method according to claim 49, wherein the analyte is selected from the group consisting of polyvinylpyrrolidone (PVP), poly(isobutylene-alt-n-octyl maleimide) (POI), picrocrocin, curcumin, and components of Chinese tea.

54. A method of functionalizing a polymer comprising:
providing a polymer;
providing a nanocage of Formula (I): 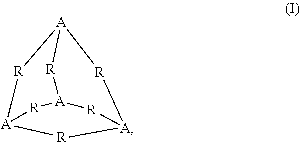
wherein each A is independently selected and has the formula 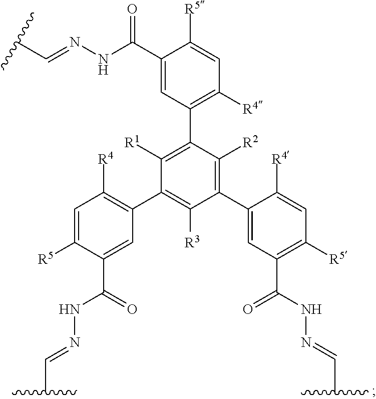
is the point of attachment of A to R;
each R is independently selected and has the formula 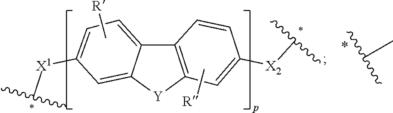
indicates the point of attachment of R to A;
R′ is H or C₁₋₂₀ alkyl;
R″ is H or C₁₋₂₀ alkyl;
R¹, R², and R³ are each independently selected from the group consisting of H, halogen, —OH, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁴, R^{4′}, and R^{4″} are each independently selected from the group consisting of H, halogen, C₁₋₂₀ alkyl, C₂₋₂₀ alkenyl, C₂₋₂₀ alkynyl, perfluorinated C₁₋₂₀ alkyl, aryl, heteroaryl, heterocyclyl, —OH, —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated C₁₋₂₀ alkyl, —CONH-aryl, —CONH-heteroaryl, and —CONH-heterocyclyl, wherein each C₁₋₂₀ alkyl, C₂₋₂₀ alkenyl, C₂₋₂₀ alkynyl, perfluorinated C₁₋₂₀ alkyl, aryl, heteroaryl, heterocyclyl, —OC₁₋₂₀ alkyl, —OC₂₋₂₀ alkenyl, —OC₂₋₂₀ alkynyl, —O-perfluorinated Ciao alkyl, —Oaryl, —COOC₁₋₂₀ alkyl, —COO perfluorinated C₁₋₂₀ alkyl, —COOaryl, —CONHC₁₋₂₀ alkyl, —CONHC₂₋₂₀ alkenyl, —CONHC₂₋₂₀ alkynyl, —CONH perfluorinated Ciao alkyl, —CONH-aryl, —CONH-heteroaryl, and CONH-heterocyclyl can be optionally substituted from 1 to 3 times with a substituent selected from the group consisting of H, halogen, OH, C₁₋₆ alkyl, aryl, and arylalkyl;
R⁵, R^{5′}, and R^{5″} are each independently selected from the group consisting of H, halogen, —OH, —O(CH₂)ₙ—(OCH₂CH₂)ₘ—OC₁₋₆ alkyl, —O(CH₂)ₙ—(OCH₂CH₂)ₘ—OH, (OCH₂CH₂)ₘ—OC₁₋₆ alkyl, (OCH₂CH₂)ₘ—OH, aryl, heteroaryl, heterocyclyl, —OC₁₋₂₀ alkyl, —O-perfluorinated C₁₋₂₀ alkyl, —Oaryl, and —NR¹⁰R¹¹ wherein R¹⁰ and R¹¹ are each independently selected from the group consisting of H, C₁₋₆ alkyl, C₂₋₆ alkenyl, C₂₋₆ alkynyl, aryl, heteroarylalkyl, heterocyclylalkyl, and arylalkyl, wherein each C₁₋₆ alkyl, C₂₋₆ alkenyl, C₂₋₆ alkynyl, aryl, heteroarylalkyl, heterocyclylalkyl, and arylalkyl can be optionally substituted from 1 to 3 times with a substituent selected from the group consisting of H, C₁₋₆ alkyl, heteroarylalkyl, heterocyclylalkyl, and arylalkyl;
Y is independently selected from the group consisting of —N(R⁶)—, —S—, —O—, —C(R⁶)(R^{6′})—, and —P(R⁶)—, wherein R⁶ and R^{6′} are each independently selected at each occurrence from the group consisting of H, C₁₋₂₀ alkyl, C₂₋₂₀ alkenyl, C₂₋₂₀ alkynyl, C₃₋₈ cycloalkyl, perfluorinated C₁₋₂₀ alkyl, aryl, heteroaryl, and heterocyclyl, wherein C₁₋₂₀ alkyl, C₂₋₂₀ alkenyl, C₂₋₂₀ alkynyl, C₃₋₈ cycloalkyl, perfluorinated C₁₋₂₀ alkyl, awl, heteroaryl, and heterocyclyl can be substituted from 1 to 3 times with a substituent selected from the group consisting of —OC₁₋₂₀ alkyl, —OC₂₋₂₀ alkenyl, —OC₂₋₂₀ alkynyl, —O-perfluorinated C₁₋₂₀ alkyl, —Oaryl, —COOC₁₋₂₀ alkyl, —COO perfluorinated Ciao alkyl, —COOaryl, —CONHC₁₋₂₀ alkyl, —CONHC₂₋₂₀ alkenyl, —CONHC₂₋₂₀ alkynyl, —CONH perfluorinated Ciao alkyl, —CONH-aryl, —CONH-heteroaryl, and CONH-heterocyclyl;
X¹ and X² are absent or are independently selected from the group consisting of C₁₋₆ alkylene, C₃₋₈ cycloalkylene, and arylene, wherein C₁₋₆ alkylene, C₃₋₈ cycloalkylene, and arylene can be optionally substituted from 1 to 3 times with H or C₁₋₆ alkyl;
p is 1 to 5;
n is 1 to 10; and
m is 1 to 50;
providing a functionalizing reagent;
reacting the polymer with the functionalizing reagent within the nanocage having a Formula (I) to produce a functionalized polymer.

55.-64. (canceled)
